(19) United States
(12) Patent Application Publication
(10) Pub. No.: US 2025/0381284 A1
(43) Pub. Date: Dec. 18, 2025

(54) PRNP RNAi AGENTS

(71) Applicant: ELI LILLY AND COMPANY, Indianapolis, IN (US)

(72) Inventors: Riazul ALAM, Fishers, IN (US); Guillermo S. CORTEZ, Indianapolis, IN (US); Kevin Patrick CRAIG, Wilmington, MA (US); Yan DING, Carmel, IN (US); Mahn Suk KYE, Boston, MA (US); Shirley So Ling LEE, Boston, MA (US); Katherine Marie MARSHALL, Hingham, MA (US); Michael Payam MOAZAMI, Boston, MA (US); Yan WANG, Carmel, IN (US); Aaron D. WROBLESKI, Indianapolis, IN (US)

(21) Appl. No.: 19/240,131

(22) Filed: Jun. 17, 2025

Related U.S. Application Data
(60) Provisional application No. 63/661,195, filed on Jun. 18, 2024.
(60) Provisional application No. 63/708,275, filed on Oct. 17, 2024.

(51) Int. Cl. A61K 47/68; C07K 16/28; C12N 15/113

(52) U.S. Cl. CPC A61K 47/6807; A61K 47/6849; A61K 47/6889; C07K 16/2881; C12N 15/113; C07K 2317/92; C12N 2310/14; C12N 2310/31; C12N 2310/3513

(57) ABSTRACT

Provided herein are PRNP RNAi agents and compositions comprising a PRNP RNAi agent. Also provided herein are methods of using the PRNP RNAi agents or compositions comprising a PRNP RNAi agent in reducing PRNP expression and/or treating prion diseases.

### SEQUENCE LISTING

The present application is being filed along with a Sequence Listing in ST.26 XML format. The Sequence Listing is provided as a file titled “31038_US” created Jun. 9, 2025, and is 272 kilobytes in size. The Sequence Listing information in the ST.26 XML format is incorporated herein by reference in its entirety.

### BACKGROUND

Prion diseases also known as transmissible spongiform encephalopathies are a group of fatal neurodegenerative disorders that affect both humans and animals. These diseases typically progress quickly with rapid cognitive decline and is fatal within a few months of symptom onset. Prion protein is encoded by the gene PRNP and contains a highly unstable region of five tandem octapeptide repeats. In pathological conditions, prion protein misfolds and recruits other prion protein molecules to misfold. The misfolded proteins may spread from cell to cell and in some cases, to a new host. Mutations in the repeat region as well as elsewhere in the PRNP gene have been associated with Creutzfeldt-Jakob disease, fatal familial insomnia, Gerstmann-Straussler disease, Huntington disease-like 1, and kuru.

The blood brain barrier (BBB) is a selective semipermeable border of capillary endothelial cells that prevents solutes, including pathogens, from passing into the central nervous system (CNS). The BBB allows the passage of some small molecules by passive diffusion and the cells of BBB actively transport metabolic products crucial to neural function such as glucose and amino acids across the barrier using specific transport proteins. The BBB has neuroprotective function by tightly controlling access to the brain; but it also impedes access of therapeutic agents to CNS. Antibodies directed to transferrin receptor (“TfR”) have been used for modulating BBB transport. However, attempts at using anti-TfR antibodies to shuttle therapeutic agents across the BBB have proven challenging. To date, there are no approved TfR shuttles or conjugates for the treatment of CNS diseases in the United States.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

There remains a need for therapeutic agents that can inhibit or adjust the expression of PRNP for treating prion diseases e.g., by utilizing RNAi. There is also need for conjugates that can deliver PRNP RNAi agent across the BBB into the CNS.

### SUMMARY OF INVENTION

Provided herein are PRNP RNAi agents capable of crossing BBB and compositions comprising such a PRNP RNAi agent. Also provided herein are methods of using such PRNP RNAi agents or compositions comprising a PRNP RNAi agent for reducing PRNP expression, and/or treating prion diseases in a subject.

In one aspect, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain (“human TfR binding protein”); and wherein n is an integer of 1 to 3. In some embodiments, n is 1. In some embodiments, n is 2. In some embodiments, n is 3.

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3 or 66, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, VH comprises SEQ ID NO: 7 or 67, and VL comprises SEQ ID NO: 8. In some embodiments, VH comprises a sequence having at least 95% sequence identity to SEQ ID NO: 7 or 67, and VL comprises a sequence having at least 95% sequence identity to SEQ ID NO: 8. Exemplary sequences of human TfR binding domains and proteins are provided in Table 1a and 1b.

In some embodiments, L is a SMCC linker, OD linker, or MSPT linker (see Table 3). In some embodiments, L is a SMCC linker or MSPT linker in Table 3.

Also provided herein are PRNP RNAi agents comprising a double stranded RNA (dsRNA) comprising a sense stand and an antisense strand, and the dsRNA is any dsRNA in Table 4a or 4b.

Exemplary unmodified sense strand and antisense strand sequences of dsRNA targeting human PRNP mRNA are provided in Table 4a. In some embodiments, the sense strand and the antisense strand comprise a pair of nucleic acid sequences selected from the group consisting of:
(a) the sense strand comprises SEQ ID NO: 25, and the antisense strand comprises SEQ ID NO: 26,
(b) the sense strand comprises SEQ ID NO: 27, and the antisense strand comprises SEQ ID NO: 28,
(c) the sense strand comprises SEQ ID NO: 29, and the antisense strand comprises SEQ ID NO: 30,
(d) the sense strand comprises SEQ ID NO: 31, and the antisense strand comprises SEQ ID NO: 32,
(e) the sense strand comprises SEQ ID NO: 33, and the antisense strand comprises SEQ ID NO: 34,
(f) the sense strand comprises SEQ ID NO: 35, and the antisense strand comprises SEQ ID NO: 36,
(g) the sense strand comprises SEQ ID NO: 37, and the antisense strand comprises SEQ ID NO: 38,
(h) the sense strand comprises SEQ ID NO: 39, and the antisense strand comprises SEQ ID NO: 40, and
(i) the sense strand comprises SEQ ID NO: 41, and the antisense strand comprises SEQ ID NO: 42,
   wherein optionally one or more nucleotides of the sense strand and the antisense strand are independently modified nucleotides, and wherein optionally one or more internucleotide linkages of the sense strand and the antisense strand are modified internucleotide linkages.

The dsRNA can include modifications. The modifications can be made to one or more nucleotides of the sense and/or antisense strand or to the internucleotide linkages. In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide.

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human PRNP mRNA are provided in Table 4b. In some embodiments, the sense strand and the antisense strand comprise a pair of nucleic acid sequences selected from the group consisting of:
(a) the sense strand comprises SEQ ID NO: 43, and the antisense strand comprises SEQ ID NO: 44,
(b) the sense strand comprises SEQ ID NO: 45, and the antisense strand comprises SEQ ID NO: 46,
(c) the sense strand comprises SEQ ID NO: 47, and the antisense strand comprises SEQ ID NO: 48,
(d) the sense strand comprises SEQ ID NO: 49, and the antisense strand comprises SEQ ID NO: 50,
(e) the sense strand comprises SEQ ID NO: 51, and the antisense strand comprises SEQ ID NO: 52,
(f) the sense strand comprises SEQ ID NO: 53, and the antisense strand comprises SEQ ID NO: 54,
(g) the sense strand comprises SEQ ID NO: 55, and the antisense strand comprises SEQ ID NO: 56,
(h) the sense strand comprises SEQ ID NO: 57, and the antisense strand comprises SEQ ID NO: 58, and
(i) the sense strand comprises SEQ ID NO: 59, and the antisense strand comprises SEQ ID NO: 60.

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain, and P is selected from TBP1, TBP2, TBP3, TBP4, TBP5, TBP6, TBP7, TBP8, TBP9, TBP10, TBP11, TBP12, in Table 1b; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3).

In some embodiments, provided herein are PRNP RNAi agents comprising Formula (I): (R-L)ₙ-P, wherein R is a double stranded RNA (dsRNA) comprising a sense stand and an antisense strand, and the dsRNA is any dsRNA in Table 4a or 4b; wherein L is a linker, or absent; wherein P is a protein comprising one monovalent human TfR binding domain; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3).

In some embodiments, provided herein are PRNP RNAi agents comprising Formula (I): (R-L)ₙ-P, wherein R is a double stranded RNA (dsRNA) comprising a sense stand and an antisense strand, and the dsRNA is any dsRNA in Table 4a or 4b; wherein L is a linker, or absent; wherein P is a protein comprising one monovalent human TfR binding domain, and P is selected from TBP1, TBP2, TBP3, TBP4, TBP5, TBP6, TBP7, TBP8, TBP9, TBP10, TBP11, TBP12, in Table 1b; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3).

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain; wherein the human TfR binding domain comprises two heavy chains HC1 and HC2 and one light chain LC1, wherein HC1 comprises SEQ ID NO: 14, LC1 comprises SEQ ID NO: 10, HC2 comprises SEQ ID NO: 15, and wherein n is 1 or 2. In some embodiments, n is 1. In some embodiments, n is 2. In some embodiments, L is a linker in Table 3 (e.g., a SMCC linker or MSPT linker in Table 3).

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain; wherein the human TfR binding domain comprises two heavy chains HC1 and HC2 and one light chain LC1, wherein HC1 comprises SEQ ID NO: 16, LC1 comprises SEQ ID NO: 10, HC2 comprises SEQ ID NO: 17, and wherein n is 1. In some embodiments, L is a linker in Table 3 (e.g., a SMCC linker or MSPT linker in Table 3).

In another aspect, provided herein are methods of treating a prion disease in a patient in need thereof, and such the method comprises administering to the patient an effective amount of the PRNP RNAi agent or a pharmaceutical composition described herein. The PRNP RNAi agent or a pharmaceutical composition comprising PRNP RNAi agent can be administered to the patient intravenously or subcutaneously.

In another aspect, provided herein are PRNP RNAi agents or pharmaceutical compositions comprising a PRNP RNAi agent for use in a therapy. Also provided herein are PRNP RNAi agents or pharmaceutical compositions comprising a PRNP RNAi agent for use in the treatment of a prion disease. Also provided herein are uses of the PRNP RNAi agent in the manufacture of a medicament for treating a prion disease.

### BRIEF DESCRIPTION OF THE DRAWINGS

FIGS. 1A-1D show TBP5-MSPT-dsRNA No. 2 conjugates significant reduced PRNP mRNA in the key brain regions, compared to the PBS or nontargeting control (NTC) groups.

FIGS. 2A-2C show TBP5-SMCC-dsRNA No. 2 conjugate successfully reduced PRNP mRNA in the key brain regions, compared to the PBS or NTC groups.

FIGS. 3A-3D show multiple mTBP1-MSPT-PRNP dsRNA conjugate successfully reduced PRNP mRNA in the key brain regions, including cortex (3A), striatum (3B), brainstem (3C), and cerebellum (3D), when compared to PBS and NTC control groups.

FIG. 4 shows the durability of knock down of PRNP mRNA by TBP5-MSPT-dsRNA No. 14 up to 56 days post-dose after a single injection. Evaluated regions include cortex (CTX), striatum (STRI), cerebellum (CB), brainstem (BS), and lumber spinal cord (LSC).

### DETAILED DESCRIPTION

Provided herein are PRNP RNAi agents and compositions comprising a PRNP RNAi agent. Also provided herein are methods of using the PRNP RNAi agents or compositions comprising a PRNP RNAi agent for reducing PRNP expression and/or treating prion diseases in a subject.

In one aspect, provided herein are PRNP RNAi agents comprising Formula (I): (R-L)ₙ-P, wherein R is a double stranded RNA (dsRNA) comprising a sense stand and an antisense strand, wherein the antisense strand is complementary to PRNP mRNA; wherein L is a linker, or absent; wherein P is a protein comprising one monovalent human TfR binding domain (“human TfR binding protein”); and wherein n is an integer of 1 to 3. In some embodiments, n is 1. In some embodiments, n is 2. In some embodiments, n is 3. For clarity, when L is absent, R is directly linked to P through a direct bond.

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain; wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3 or 66, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3). For clarity, when L is absent, R is directly linked to P through a direct bond.

In some embodiments, provided herein are PRNP RNAi agents comprising Formula (I): (R-L)ₙ-P, wherein R is a double stranded RNA (dsRNA) comprising a sense stand and an antisense strand, and the antisense strand is complementary to PRNP mRNA; wherein L is a linker, or absent; wherein P is a protein comprising one monovalent human TfR binding domain, and P is selected from TBP1, TBP2, TBP3, TBP4, TBP5, TBP6, TBP7, TBP8, TBP9, TBP10, TBP11, TBP12, in Table 1b; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3). For clarity, when L is absent, R is directly linked to P through a direct bond.

In some embodiments, provided herein are PRNP RNAi agents comprising Formula (I): (R-L)ₙ-P, wherein R is a double stranded RNA (dsRNA) comprising a sense stand and an antisense strand, and the dsRNA is any dsRNA in Table 4a or 4b; wherein L is a linker, or absent; wherein P is a protein comprising one monovalent human TfR binding domain; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3). For clarity, when L is absent, R is directly linked to P through a direct bond.

In some embodiments, provided herein are PRNP RNAi agents comprising Formula (I): (R-L)ₙ-P, wherein R is a double stranded RNA (dsRNA) comprising a sense stand and an antisense strand, and the dsRNA is any dsRNA in Table 4a or 4b; wherein L is a linker, or absent; wherein P is a protein comprising one monovalent human TfR binding domain, and P is selected from TBP1, TBP2, TBP3, TBP4, TBP5, TBP6, TBP7, TBP8, TBP9, TBP10, TBP11, TBP12, in Table 1b; and wherein n is an integer of 1 to 3. In some embodiments, n is 1. In some embodiments, n is 2. In some embodiments, n is 3. In some embodiments, L is a linker in Table 3 (e.g., a SMCC linker or MSPT linker in Table 3). For clarity, when L is absent, R is directly linked to P through a direct bond.

Also provided herein are PRNP RNAi agents comprising a double stranded RNA (dsRNA) comprising a sense stand and an antisense strand, and the dsRNA is any dsRNA in Table 4a or 4b.

### Human TfR Binding Proteins

The PRNP RNAi agents described herein comprise a protein comprising one monovalent human TfR binding domain (“human TfR binding protein”). Human TfR binding protein of the PRNP RNAi agents can bind TfR on BBB and transport the dsRNA into the CNS.

Exemplary sequences of human TfR binding domains and proteins are provided in Table 1a and 1b. In some embodiments, the monovalent human TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, HCDR1 comprises SEQ ID NO: 1, HCDR2 comprises SEQ ID NO: 2, HCDR3 comprises SEQ ID NO: 3 or 66, LCDR1 comprises SEQ ID NO: 4, LCDR2 comprises SEQ ID NO: 5, and LCDR3 comprises SEQ ID NO: 6. In some embodiments, VH comprises SEQ ID NO: 7 or 67, and VL comprises SEQ ID NO: 8. In some embodiments, VH comprises a sequence having at least 95% sequence identity to SEQ ID NO: 7 or 67, and VL comprises a sequence having at least 95% sequence identity to SEQ ID NO: 8.

TABLE 1a
Exemplary sequences of human TfR binding domains and proteins

| Region | Sequence | SEQ ID NO |
|---|---|---|
| Fab 1-6 HCDR1 | SYSMN | 1 |
| (KABAT) | | |
| Fab 1-6 HCDR2 | SISSSSSYIYYADSVKG | 2 |
| (KABAT) | | |
| Fab 1, 4-6 | RHGYSNSDAFDN | 3 |
| HCDR3 | | |
| (KABAT) | | |
| Fab 1-6 LCDR1 | RASQGISHYLV | 4 |
| (KABAT) | | |
| Fab 1-6 LCDR2 | AASSLQS | 5 |
| (KABAT) | | |
| Fab 1-6 LCDR3 | LQHNSYPWT | 6 |
| (KABAT) | | |
| Fab 1, 4-6 VH | EVQLVESGGGLVKPGGSLRLSCVASGFTFSSYSMN | 7 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSS | |
| Fab 1-6 VL | DIQMTQSPSAMSASVGDRVTITCRASQGISHYLVW | 8 |
| | FQQKPGKVPKRLIYAASSLQSGVPSRFSGSGSGTEF | |
| | TLTISSLQPEDFATYYCLQHNSYPWTFGQGTKVEI | |
| | K | |
| Fab1, 5 HC | EVQLVESGGGLVKPGGSLRLSCVASGFTFSSYSMN | 9 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPSVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKC | |
| Fab1, 3, 4 LC | DIQMTQSPSAMSASVGDRVTITCRASQGISHYLVW | 10 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| Fab1-VHH HC | EVQLVESGGGLVKPGGSLRLSCVASGFTFSSYSMN | 11 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKCDKTHTGGGGQGGGGQGGGG | |
| | QGGGGQGGGGQEVQLLESGGGLVQPGGSLRLSCA | |
| | ASGRYIDETAVAWFRQAPGKGREFVAGIGGGVDI | |
| | TYYADSVKGRFTISRDNSKNTLYLQMNSLRPEDTA | |
| | VYYCGARPGRPLITSKVADLYPYWGQGTLVTVSS | |
| | PP | |
| Fab5-VHH LC | DIQMTQSPSAMSASVGDRVTITCRASQGISHYLVW | 12 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQCGNSQESVTEQDSKDSTYSLS | |
| | STLTLSKADYEKHKVYACEVTHQGLSSPVTKSFNR | |
| | GEC | |
| hIgG4 PAA | EVQLVESGGGLVKPGGSLRLSCVASGFTFSSYSMN | 13 |
| HC1 | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPXVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVYTLPPSQEEMTKNQVSLTCLVKGFYPSDIAVE | |
| | WESNGQPENNYKTTPPVLDSDGSFFLYSRLTVDKS | |
| | RWQEGNVFSCSVMHEALHNHYTQKSLSLSLG, | |
| | wherein X is S or C. | |
| OAH1 (one arm | EVQLVESGGGLVKPGGSLRLSCVASGFTFSSYSMN | 14 |
| heteromab) HC1 | WVRQAPGKGLEWVSSISSSSSYIYYADSVKGRFTIS | |
| (A378C) | RDNAKNSLYLQMNSLRAEDTAVYYCARRHGYSN | |
| | SDAFDNWGQGTLVTVSSASTKGPSVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVSTLPPSQEEMTKNQVSLMCLVYGFYPSDIXVE | |
| | WESNGQPENNYKTTPPVLDSDGSFFLYSVLTVDKS | |
| | RWQEGNVFSCSVMHEALHNHYTQKSLSLSLG, | |
| | wherein X is A or C. | |
| OAH1 HC2 | ESKYGPPCPPCPAPEAAGGPSVFLFPPKPKDTLMIS | 15 |
| (A378C) | RTPEVTCVVVDVSQEDPEVQFNWYVDGVEVHNA | |
| | KTKPREEQFNSTYRVVSVLTVLHQDWLNGKEYKC | |
| | KVSNKGLPSSIEKTISKAKGQPREPQVYTLPPSQGD | |
| | MTKNQVQLTCLVKGFYPSDIXVEWESNGQPENNY | |
| | KTTPPVLDSDGSFFLASRLTVDKSRWQEGNVFSCS | |
| | VMHEALHNHYTQKSLSLSLG, wherein X is A or C. | |
| OAH1/2 LC | DIQMTQSPSAMSASVGDRVTITCRASQGISHYLVW | 10 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| OAH2 HC1 | EVQLVESGGGLVKPGGSLRLSCVASGFTFSSYSMN | 16 |
| (S124C) | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVSTLPPSQEEMTKNQVSLMCLVYGFYPSDIAVE | |
| | WESNGQPENNYKTTPPVLDSDGSFFLYSVLTVDKS | |
| | RWQEGNVFSCSVMHEALHNHYTQKSLSLSLG | |
| OAH2 HC2 | ESKYGPPCPPCPAPEAAGGPSVFLFPPKPKDTLMIS | 17 |
| (S124C) | RTPEVTCVVVDVSQEDPEVQFNWYVDGVEVHNA | |
| | KTKPREEQFNSTYRVVSVLTVLHQDWLNGKEYKC | |
| | KVSNKGLPSSIEKTISKAKGQPREPQVYTLPPSQGD | |
| | MTKNQVQLTCLVKGFYPSDIAVEWESNGQPENNY | |
| | KTTPPVLDSDGSFFLASRLTVDKSRWQEGNVFSCS | |
| | VMHEALHNHYTQKSLSLSLG | |
| Null Arm HC | QVQLVQSGAEVKKPGSSVKVSCKASGYTFSSYAIE | 18 |
| | WVRQAPGQGLEWMGGILPGSGTINYNEKFKGRVT | |
| | ITADKSTSTAYMELSSLRSEDTAVYYCARMSSNSD | |
| | QGFDLWGQGTLVTVSSASTKGPXVFPLAPCSRSTS | |
| | ESTAALGCLVKDYFPEPVTVSWNSGALTSGVHTFP | |
| | AVLQSSGLYSLSSVVTVPSSSLGTKTYTCNVDHKP | |
| | SNTKVDKRVESKYGPPCPPCPAPEAAGGPSVFLFP | |
| | PKPKDTLMISRTPEVTCVVVDVSQEDPEVQFNWY | |
| | VDGVEVHNAKTKPREEQFNSTYRVVSVLTVLHQD | |
| | WLNGKEYKCKVSNKGLPSSIEKTISKAKGQPREPQ | |
| | VYTLPPSQEEMTKNQVSLTCLVKGFYPSDIAVEWE | |
| | SNGQPENNYKTTPPVLDSDGSFLLYSKLTVDKSR | |
| | WQEGNVFSCSVMHEALHNHYTQKSLSLSLG, | |
| | wherein X is S or C. | |
| Null Arm LC | DIQMTQSPSSLSASVGDRVTITCKASQGISRFLSWF | 19 |
| | QQKPGKAPKSLIYAVSSLVDGVPSRFSGSGSGTDF | |
| | TLTISSLQPEDFATYYCVQYNSYPYGFGGGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| Fab 2/3 HCDR3 | RHGYSNSDAFDT | 66 |
| (KABAT) | | |
| Fab2/3 VH | EVQLVESGGGLVKPGGSLRLSCVASGFTFSSYSMN | 67 |
| | WVRQAPGKGLEWVSSISSSSSYIYYADSVKGRFTIS | |
| | RDNAKNSLYLQMNSLRAEDTAVYYCARRHGYSN | |
| | SDAFDTWGQGTLVTVSS | |
| Fab2/3 HC | EVQLVESGGGLVKPGGSLRLSCVASGFTFSSYSMN | 68 |
| | WVRQAPGKGLEWVSSISSSSSYIYYADSVKGRFTIS | |
| | RDNAKNSLYLQMNSLRAEDTAVYYCARRHGYSN | |
| | SDAFDTWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRV | |
| Fab2 LC (no | DIQMTQSPSAMSASVGDRVTITCRASQGISHYLVW | 69 |
| disulfide) | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GE | |
| Fab2-VHH HC | EVQLVESGGGLVKPGGSLRLSCVASGFTFSSYSMN | 70 |
| | WVRQAPGKGLEWVSSISSSSSYIYYADSVKGRFTIS | |
| | RDNAKNSLYLQMNSLRAEDTAVYYCARRHGYSN | |
| | SDAFDTWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVGGGGQGGGGQGGGGQGGGGQGG | |
| | GGQEVQLLESGGGLVQPGGSLRLSCAASGRYIDET | |
| | AVAWFRQAPGKGREFVAGIGGGVDITYYADSVKG | |
| | RFTISRDNSKNTLYLQMNSLRPEDTAVYYCGARPG | |
| | RPLITSKVADLYPYWGQGTLVTVSSPP | |
| Fab6-VHH HC | EVQLVESGGGLVKPGGSLRLSCVASGFTFSSYSMN | 86 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKSSDKTHTGGGGQGGGGQGGG | |
| | GQGGGGQGGGGQEVQLLESGGGLVQPGGSLRLSC | |
| | AASGRYIDETAVAWFRQAPGKGREFVAGIGGGVD | |
| | ITYYADSVKGRFTISRDNSKNTLYLQMNSLRPEDT | |
| | AVYYCGARPGRPLITSKVADLYPYWGQGTLVTVS | |
| | SC | |
| Fab4-VHH HC | EVQLVESGGGLVKPGGSLRLSCVASGFTFSSYSMN | 87 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKSCDKTHTGGGGQGGGGQGGG | |
| | GQGGGGQGGGGQEVQLLESGGGLVQPGGSLRLSC | |
| | AASGRYIDETAVAWFRQAPGKGREFVAGIGGGVD | |
| | ITYYADSVKGRFTISRDNSKNTLYLQMNSLRPEDT | |
| | AVYYCGARPGRPLITSKVADLYPYWGQGTLVTVS | |
| | SPP | |
| Fab4 HC | EVQLVESGGGLVKPGGSLRLSCVASGFTFSSYSMN | 88 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKSCD | |
| Fab6-VHH LC | DIQMTQSPSAMSASVGDRVTITCRASQGISHYLVW | 89 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEV | |

TABLE 1b
Exemplary sequences of human TfR binding proteins

| Human TfR binding | | | | |
|---|---|---|---|---|
| protein (TBP) | HC1 | LC1 | HC2 | LC2 |
| TBP1 (Fab1) | SEQ ID NO: 9 | SEQ ID NO: 10 | N/A | N/A |
| TBP2 (Fab5-VHH) | SEQ ID NO: 11 | SEQ ID NO: 12 | N/A | N/A |
| TBP3 (Fab 1- | SEQ ID NO: 13 | SEQ ID NO: 10 | SEQ ID NO: 18 | SEQ ID NO: 19 |
| Heterodimeric Ab) | | | | |
| TBP4 (Fab1-One | SEQ ID NO: 14 | SEQ ID NO: 10 | SEQ ID NO: 15 | N/A |
| Arm Heteromab 1, | | | | |
| A378C on HC2) | | | | |
| TBP5 (Fab1-One | SEQ ID NO: 16 | SEQ ID NO: 10 | SEQ ID NO: 17 | N/A |
| Arm Heteromab 2, | | | | |
| S124C) | | | | |
| TBP6 (Fab1-VHH) | SEQ ID NO: 11 | SEQ ID NO: 10 | N/A | N/A |
| TBP7 (Fab2) | SEQ ID NO: 68 | SEQ ID NO: 69 | N/A | N/A |
| TBP8 (Fab2-VHH) | SEQ ID NO: 70 | SEQ ID NO: 69 | N/A | N/A |
| TBP9 (Fab3) | SEQ ID NO: 68 | SEQ ID NO: 10 | N/A | N/A |
| TBP10 (Fab6-VHH) | SEQ ID NO: 86 | SEQ ID NO: 89 | N/A | N/A |
| TBP11 (Fab4-VHH) | SEQ ID NO: 87 | SEQ ID NO: 10 | N/A | N/A |
| TBP12 (Fab4) | SEQ ID NO: 88 | SEQ ID NO: 10 | N/A | N/A |

In some embodiments, the monovalent human TfR binding domain is an antibody fragment, e.g., Fab, scFv, Fv, or scFab (single chain Fab). In some embodiments, the monovalent human TfR binding domain is Fab. In some embodiments, the human TfR binding domain further comprises a heavy chain constant region and/or a light chain constant region.

In some embodiments, the human TfR binding protein further comprises a half-life extender, e.g., an immunoglobulin Fc region or a VHH that binds human serum albumin (HSA).

In some embodiments, the human TfR binding protein further comprises an immunoglobulin Fc region, e.g., a modified human IgG4 Fc region, or a modified human IgG1 Fc region. In some embodiments, the human TfR binding protein further comprises a modified human IgG4 Fc region comprising proline at residue 228, and alanine at residues 234 and 235 (all residues are numbered according to the EU Index numbering, also called hIgG4PAA Fc region). In some embodiments, the human TfR binding protein further comprises a modified human IgG1 Fc region comprising alanine at residues 234, 235, and 329, serine at position 265, aspartic acid at position 436 (all residues are numbered according to the EU Index numbering, also called hIgG1 effector null or hIgGIEN Fc region).

In some embodiments, the human TfR binding protein further comprise a VHH that binds human HSA. In some embodiments, the VHH also binds mouse, rat, and/or cynomolgus monkey albumin. An exemplary VHH that binds human HSA is shown in Table 2. In some embodiments, such a VHH comprises CDR1 comprising SEQ ID NO: 20, CDR2 comprising SEQ ID NO: 21, and CDR3 comprising SEQ ID NO: 22. In some embodiments, such a VHH comprises SEQ ID NO: 23. In some embodiments, the VHH is linked to the TfR binding domain through a peptide linker, e.g., (GGGGQ) 4 (SEQ ID NO: 24). In some embodiments, the VHH is linked to the C-terminus of the TfR binding domain.

TABLE 2
Exemplary sequences of VHH that binds human serum albumin (HSA)

| Region | Sequence | SEQ ID NO |
|---|---|---|
| CDR1 | ETAVA | 20 |
| (KABAT) | | |
| CDR2 | GIGGGVDITYYADSVKG | 21 |
| (KABAT) | | |
| CDR3 | RPGRPLITSKVADLYPY | 22 |
| (KABAT) | | |
| VHH full | EVQLLESGGGLVQPGGSLRLSCAASGRYIDETAV | 23 |
| length | AWFRQAPGKGREFVAGIGGGVDITYYADSVKGR | |
| | FTISRDNSKNTLYLQMNSLRPEDTAVYYCGARPG | |
| | RPLITSKVADLYPYWGQGTLVTVSSPP | |
| Optional | GGGGQGGGGQGGGGQGGGGQ | 24 |
| linker | | |

In some embodiments, the human TfR binding protein is heterodimeric antibody that comprises a first arm comprising one monovalent human TfR binding domain and a second arm that is a null arm, e.g., an arm that does not bind any known human target (e.g., an isotype arm). Heterodimeric antibodies such as heteromab, orthomab or duobody have been described in WO2014150973, WO2016118742, WO2018118616, and WO2011131746. In some embodiments, the first arm comprises any monovalent human TfR binding domain described herein. In some embodiments, the second arm is a null arm that does not bind any known human target (e.g., an isotype arm) comprises the sequences in Table 1a. In some embodiments, the second arm comprises a heavy chain (HC) and a light chain (LC), wherein the HC comprises SEQ ID NO: 18, and the LC comprises SEQ ID NO: 19.

In some embodiments, the human TfR binding protein comprises heterodimeric mutations. In some embodiments, the human TfR binding protein comprises a modified Fc region comprising a first Fc CH3 domain comprising serine at residue 349, methionine at residue 366, tyrosine at residue 370, and valine at residue 409, and a second Fc CH3 domain comprising glycine at residue 356, aspartic acid at residue 357, glutamine at residue 364 and alanine at residue 407 (all residues are numbered according to the EU Index numbering). In some embodiments, the human TfR binding protein comprises a modified Fc region comprising a first Fc CH3 domain comprising leucine at residue 405, and a second Fc CH3 domain comprising arginine at residue 409 (all residues are numbered according to the EU Index numbering).

In some embodiments, the human TfR binding protein comprises one or more native cysteine residues, which can be used for conjugation. For example, in some embodiments, the human TfR binding protein comprises a native cysteine at position 220 of the light chain and/or a native cysteine at position 226 of the heavy chain, which can be used for conjugation (all residues according to the EU Index numbering).

In some embodiments, the human TfR binding protein comprises engineered cysteine residues for conjugation. The approach of including engineered cysteines as a means for conjugation has been described in WO 2018/232088. In some embodiments, the human TfR binding protein comprises a heavy chain comprising one or more cysteines at the following residues: 124, 157, 162, 262, 373, 375, 378, 397, 415 (all residues according to the EU Index numbering). In some embodiments, the human TfR binding protein comprises a light chain (e.g., a kappa light chain) comprising one or more cysteines at the following residues: 156, 171, 191, 193, 202, 208 (all residues according to the EU Index numbering). In some embodiments, the human TfR binding protein comprises a heavy chain constant region comprising cysteine at residue 124 (according to the EU Index numbering). In some embodiments, the human TfR binding protein comprises a light chain constant region comprising cysteine at residue 156 (according to the EU Index numbering). In some embodiments, the human TfR binding protein comprises an immunoglobulin Fc region comprising cysteine at residue 378 (according to the EU Index numbering).

In some embodiments, the human TfR binding protein is any one of the human TfR binding proteins in Table 1b, e.g., TBP1, TBP2, TBP3, TBP4, TBP5, TBP6, TBP7, TBP8, TBP9, TBP10, TBP11, TBP12.

In some embodiments, the human TfR binding protein has a Fab format, e.g., TBP1, TBP7, TBP9, TBP12. In some embodiments, the human TfR binding protein comprises one HC and one LC, and wherein the HC comprises SEQ ID NO: 9, 68, or 88, and the LC comprises SEQ ID NO: 10, 12, or 69. In some embodiments, the human TfR binding protein comprises one HC and one LC, and wherein the HC comprises SEQ ID NO: 9 and the LC comprises SEQ ID NO: 10. In some embodiments, the human TfR binding protein comprises one HC and one LC, and wherein the HC comprises SEQ ID NO: 9 and the LC comprises SEQ ID NO: 12. In some embodiments, the human TfR binding protein comprises one HC and one LC, and wherein the HC comprises SEQ ID NO: 9 and the LC comprises SEQ ID NO: 69. In some embodiments, the human TfR binding protein comprises one HC and one LC, and wherein the HC comprises SEQ ID NO: 68 and the LC comprises SEQ ID NO: 69. In some embodiments, the human TfR binding protein comprises one HC and one LC, and wherein the HC comprises SEQ ID NO: 68 and the LC comprises SEQ ID NO: 10. In some embodiments, the human TfR binding protein comprises one HC and one LC, and wherein the HC comprises SEQ ID NO: 68 and the LC comprises SEQ ID NO: 12. In some embodiments, the human TfR binding protein comprises one HC and one LC, and wherein the HC comprises SEQ ID NO: 88 and the LC comprises SEQ ID NO: 10.

In some embodiments, the human TfR binding protein has a Fab-VHH format, e.g., TBP2, TBP6, TBP8, TBP10, TBP11. In some embodiments, the human TfR binding protein comprises one HC and one LC, and wherein the HC comprises SEQ ID NO: 11, 70, or 87, and the LC comprises SEQ ID NO: 10, 12, or 69. In some embodiments, the human TfR binding proteins comprises one HC and one LC, wherein the HC comprises SEQ ID NO: 11 and the LC comprises SEQ ID NO: 12. In some embodiments, the human TfR binding proteins comprises one HC and one LC, wherein the HC comprises SEQ ID NO: 11 and the LC comprises SEQ ID NO: 10. In some embodiments, the human TfR binding proteins comprises one HC and one LC, wherein the HC comprises SEQ ID NO: 11 and the LC comprises SEQ ID NO: 69. In some embodiments, the human TfR binding proteins comprises one HC and one LC, wherein the HC comprises SEQ ID NO: 70 and the LC comprises SEQ ID NO: 69. In some embodiments, the human TfR binding proteins comprises one HC and one LC, wherein the HC comprises SEQ ID NO: 70 and the LC comprises SEQ ID NO: 10. In some embodiments, the human TfR binding proteins comprises one HC and one LC, wherein the HC comprises SEQ ID NO: 70 and the LC comprises SEQ ID NO: 12. In some embodiments, the human TfR binding proteins comprises one HC and one LC, wherein the HC comprises SEQ ID NO: 86 and the LC comprises SEQ ID NO: 89. In some embodiments, the human TfR binding proteins comprises one HC and one LC, wherein the HC comprises SEQ ID NO: 87 and the LC comprises SEQ ID NO: 10.

In some embodiments, the human TfR binding protein has a heterodimeric antibody format, e.g., TBP3. In some embodiments, the human TfR binding protein comprises two heavy chains HC1 and HC2 and two light chains LC1 and LC2, wherein HC1 comprises SEQ ID NO: 13, LC1 comprises SEQ ID NO: 10, HC2 comprises SEQ ID NO: 18, and LC2 comprises SEQ ID NO: 19.

In some embodiments, the human TfR binding protein has a one arm heteromab format, e.g., TBP4 or TBP5. In some embodiments, the human TfR binding protein comprises two heavy chains HC1 and HC2 and one light chain LC1, wherein HC1 comprises SEQ ID NO: 14, LC1 comprises SEQ ID NO: 10, HC2 comprises SEQ ID NO: 15. In some embodiments, provided herein are human TfR binding proteins comprise two heavy chains HC1 and HC2 and one light chain LC1, wherein HC1 comprises SEQ ID NO: 16, LC1 comprises SEQ ID NO: 10, HC2 comprises SEQ ID NO: 17.

The human TfR binding proteins described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the TfR binding proteins) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of the TfR binding proteins described herein. According to some embodiments, a host cell may be stably or transiently transfected, transformed, transduced or infected with an expression vector expressing HC polypeptides and an expression vector expressing LC polypeptides of the TfR binding proteins described herein. In some embodiments, a host cell may be stably or transiently transfected, transformed, transduced or infected with an expression vector expressing HC and LC polypeptides of the TfR binding proteins described herein. The TfR binding proteins may be produced in mammalian cells such as CHO, NS0, HEK293 or COS cells according to techniques well known in the art.

Medium, into which the TfR binding proteins has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

### Mouse TfR Binding Proteins

Some conjugates used in the Examples below comprise a protein comprising one monovalent mouse TfR binding domain (“mouse TfR binding proteins” or mTBP). Exemplary sequences of mouse TfR binding proteins are provided in Table 1c and 1d. Such conjugates comprising a mouse TfR binding protein can serve as surrogate molecules in mouse models.

TABLE 1c
Exemplary sequences of mouse TfR binding proteins

| Region | Sequence | SEQ ID NO |
|---|---|---|
| HCDR1 | GSYWIC | 71 |
| (KABAT) | | |
| HCDR2 | CIYSTSGGRTYYASWVKG | 72 |
| (KABAT) | | |
| HCDR3 | GDDSISDAYFDL | 73 |
| (KABAT) | | |
| LCDR1 | QSSQSVYNNNRLA | 74 |
| (KABAT) | | |
| LCDR2 | DASTLAS | 75 |
| (KABAT) | | |
| LCDR3 | QGTYFSSGWSWA | 76 |
| (KABAT) | | |
| VH | QSLEESGGDLVKPEGSLTLTCTASGFSFSGSYWI | 77 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSS | |
| VL | ALDMTQTASPVSAAVGGTVTINCQSSQSVYNN | 78 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVK | |
| OAH1-HC1 | QSLEESGGDLVKPEGSLTLTCTASGFSFSGSYWI | 79 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPCVFPL | |
| | APCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPP | |
| | VLDSDGSFFLYSRLTVDKSRWQEGNVFSCSVM | |
| | HEALHNHYTQKSLSLSLG | |
| OAH2-HC1 | QSLEESGGDLVKPEGSLTLTCTASGFSFSGSYWI | 80 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPSVFPLA | |
| | PCSRSTSESTAALGCLVKDYFPEPVTVSWNSGA | |
| | LTSGVHTFPAVLQSSGLYSLSSVVTVPSSSLGTK | |
| | TYTCNVDHKPSNTKVDKRVESKYGPPCPPCPAP | |
| | EAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDICVEWESNGQPENNYKTTPP | |
| | VLDSDGSFFLYSRLTVDKSRWQEGNVFSCSVM | |
| | HEALHNHYTQKSLSLSLG | |
| LC1 | ALDMTQTASPVSAAVGGTVTINCQSSQSVYNN | 81 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVKRTVAAPSVFIFPPSDEQLK | |
| | SGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| OAH1-HC2 | ESKYGPPCPPCPAPEAAGGPSVFLFPPKPKDTLM | 82 |
| | ISRTPEVTCVVVDVSQEDPEVQFNWYVDGVEV | |
| | HNAKTKPREEQFNSTYRVVSVLTVLHQDWLNG | |
| | KEYKCKVSNKGLPSSIEKTISKAKGQPREPQVYT | |
| | LPPSQEEMTKNQVSLTCLVKGFYPSDIAVEWES | |
| | NGQPENNYKTTPPVLDSDGSFLLYSKLTVDKSR | |
| | WQEGNVFSCSVMHEALHNHYTQKSLSLSLG | |
| OAH2-HC2 | ESKYGPPCPPCPAPEAAGGPSVFLFPPKPKDTLM | 83 |
| | ISRTPEVTCVVVDVSQEDPEVQFNWYVDGVEV | |
| | HNAKTKPREEQFNSTYRVVSVLTVLHQDWLNG | |
| | KEYKCKVSNKGLPSSIEKTISKAKGQPREPQVYT | |
| | LPPSQEEMTKNQVSLTCLVKGFYPSDICVEWES | |
| | NGQPENNYKTTPPVLDSDGSFLLYSKLTVDKSR | |
| | WQEGNVFSCSVMHEALHNHYTQKSLSLSLG | |
| Fab-HC | QSLEESGGDLVKPEGSLTLTCTASGFSFSGSYWI | 84 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPCVFPL | |
| | APSSKSTSGGTAALGCLVKDYFPEPVTVSWNSG | |
| | ALTSGVHTFPAVLQSSGLYSLSSVVTVPSSSLGT | |
| | QTYICNVNHKPSNTKVDKKVEPKSCDKTH | |
| Fab-VHH HC | QSLEESGGDLVKPEGSLTLTCTASGFSFSGSYWI | 85 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPCVFPL | |
| | APSSKSTSGGTAALGCLVKDYFPEPVTVSWNSG | |
| | ALTSGVHTFPAVLQSSGLYSLSSVVTVPSSSLGT | |
| | QTYICNVNHKPSNTKVDKKVEPKSCDKTHLEVL | |
| | FQGPGGGGQGGGGQGGGGQGGGGQGGGGQE | |
| | VQLLESGGGLVQPGGSLRLSCAASGRYIDETAV | |
| | AWFRQAPGKGREFVAGIGGGVDITYYADSVKG | |
| | RFTISRDNSKNTLYLQMNSLRPEDTAVYYCAAR | |
| | PGRPLITSKVADLYPYWGQGTLVTVSSPP | |

TABLE 1d
Exemplary sequences of mouse TfR binding proteins

| Mouse TfR | | | |
|---|---|---|---|
| binding | | | |
| protein (TBP) | HC1 | LC1 | HC2 |
| mTBP1 | SEQ ID NO: 79 | SEQ ID NO: 81 | SEQ ID NO: 82 |
| (OAH1) | | | |
| mTBP2 | SEQ ID NO: 80 | SEQ ID NO: 81 | SEQ ID NO: 83 |
| (OAH2) | | | |
| mTBP3 | SEQ ID NO: 84 | SEQ ID NO: 81 | N/A |
| (Fab) | | | |
| mTBP4 | SEQ ID NO: 85 | SEQ ID NO: 81 | N/A |
| (Fab-VHH) | | | |

### Linker

In some embodiments, the PRNP RNAi agents described herein comprises a linker that links the human TfR binding protein to the dsRNA. Exemplary linker structures are shown in Table 3. In some embodiments, the linker is a SMCC linker, OD linker, or MSPT linker. In some embodiments, the linker is a SMCC linker. In some embodiments, the linker is a MSPT linker.

TABLE 3
Exemplary linker structures

| Link- | |
|---|---|
| er | Structure** |
| 1 | SMCC linker 1* |
| | 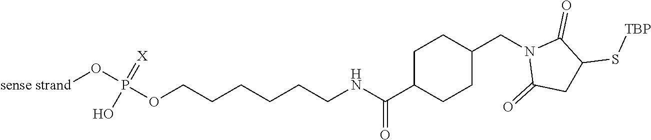 |
| 2 | SMCC linker 2 |
| | 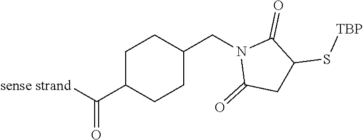 |
| 3 | Hydrolyzed ring open form of SMCC linker 1* |
| | 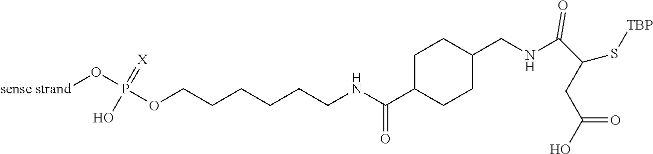 |
| 4 | Hydrolyzed ring open form of SMCC linker 2 |
| | 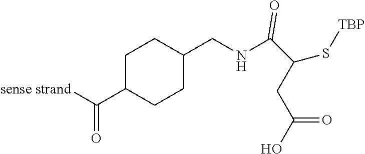 |
| 5 | Mal-Tet-TCO linker 1* |
| | 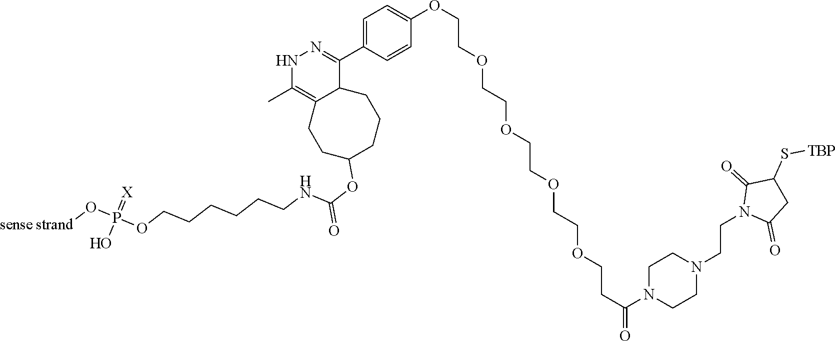 |
| 6 | Mal-Tet-TCO linker 2 |
| | 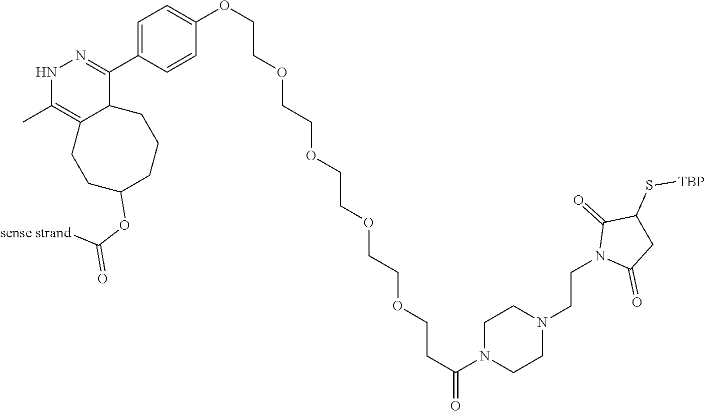 |
| 7 | GDM linker 1* |
| | 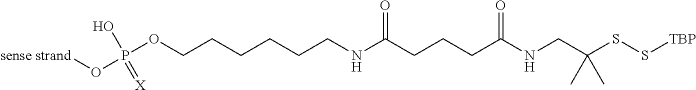 |
| 8 | GDM linker 2 |
| | 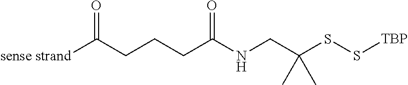 |
| 9 | 3′-OD linker* |
| | 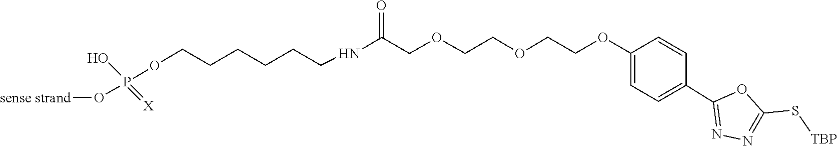 |
| 10 | 3′-MSPT linker* |
| | 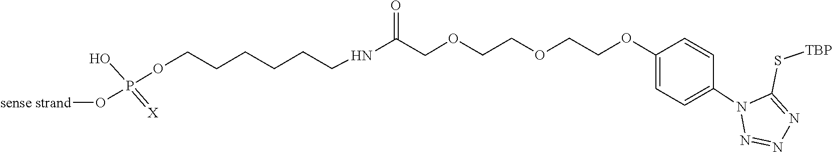 |
| 11 | 5′-OD linker |
| | 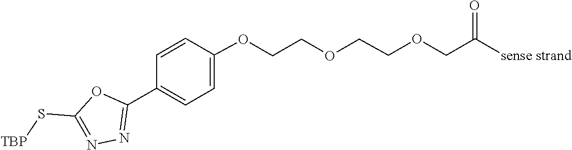 |
| 12 | 5′-MSPT linker |
| | 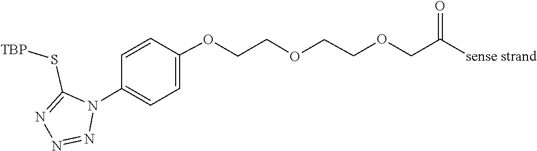 |

*Note- X is O or S
**One skilled in the art will recognize that the sulfur connection to TBP may be considered as part of the TBP.

dsRNA

The PRNP RNAi agents described herein comprise a double stranded RNA (dsRNA) comprising a sense stand and an antisense strand, and wherein the antisense strand is complementary to PRNP mRNA. After the antisense strand of the dsRNA is incorporated into the RNA-induced silencing complex (RISC), the RISC can bind and degrade target PRNP mRNA.

In some embodiments, the sense strand and the antisense strand of the dsRNA are each 15-30 nucleotides in length, e.g., 20-25 nucleotides in length. In some embodiments, the dsRNA has a sense strand of 21 nucleotides and an antisense strand of 23 nucleotides. In some embodiments, the sense strand and antisense strand of the dsRNA may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

Exemplary unmodified sense strand and antisense strand sequences of dsRNA targeting human PRNP mRNA are provided in Table 4a.

In some embodiments, the sense strand comprises SEQ ID NO: 25, and the antisense strand comprises SEQ ID NO: 26. In some embodiments, the sense strand comprises SEQ ID NO: 27, and the antisense strand comprises SEQ ID NO: 28. In some embodiments, the sense strand comprises SEQ ID NO: 29, and the antisense strand comprises SEQ ID NO: 30. In some embodiments, the sense strand comprises SEQ ID NO: 31, and the antisense strand comprises SEQ ID NO: 32. In some embodiments, the sense strand comprises SEQ ID NO: 33, and the antisense strand comprises SEQ ID NO: 34. In some embodiments, the sense strand comprises SEQ ID NO: 35, and the antisense strand comprises SEQ ID NO: 36. In some embodiments, the sense strand comprises SEQ ID NO: 37, and the antisense strand comprises SEQ ID NO: 38. In some embodiments, the sense strand comprises SEQ ID NO: 39, and the antisense strand comprises SEQ ID NO: 40. In some embodiments, the sense strand comprises SEQ ID NO: 41, and the antisense strand comprises SEQ ID NO: 42.

TABLE 4a
Unmodified sequences of dsRNA targeting human PRNP mRNA

| | | | | | Start |
|---|---|---|---|---|---|
| | | | | | position of |
| | | | | | antisense |
| | | | | | strand target |
| | | | | | region of |
| | | | | | human PRNP |
| | | SEQ | | SEQ | transcript |
| dsRNA | Sense Strand | ID | Antisense Strand | ID | NM_000311.5 |
| No. | (5′ to 3′) | NO | (5′ to 3′) | NO | (SEQ ID NO: 61) |
| 1 | CACGACUGCGUCAAU | 25 | UGUGAUAUUGACGCAGUC | 26 | 594 |
| | AUCACA | | GUGCA | | |
| 3 | AUUACUUUUCUGCAA | 27 | UUAACAUUGCAGAAAAG | 28 | 2154 |
| | UGUUAA | | UAAUAC | | |
| 4 | UACAAUGUGCACUGA | 29 | UACGAUUCAGUGCACAUU | 30 | 2358 |
| | AUCGUA | | GUAAG | | |
| 5 | GAAGUGGAAAAAGAA | 31 | UAGAAUUUCUUUUUCCAC | 32 | 2070 |
| | AUUCUA | | UUCAA | | |
| 6 | CACCAUCUUUCUAAU | 33 | UAAAAGAUUAGAAAGAU | 34 | 952 |
| | CUUUUA | | GGUGAA | | |
| 7 | CUAAUGCAUUAACUU | 35 | UUACAAAAGUUAAUGCA | 36 | 2287 |
| | UUGUAA | | UUAGAC | | |
| 8 | GCUCAGUAUACUAAU | 37 | UAGGGCAUUAGUAUACU | 38 | 1306 |
| | GCCCUA | | GAGCUC | | |
| 9 | UUCCCUGAAUUGUUU | 39 | UAUAUCAAACAAUUCAGG | 40 | 2115 |
| | GAUAUA | | GAAUA | | |
| 10 | UGAUGUUUUACUUUU | 41 | UCUGUGAAAAGUAAAAC | 42 | 1511 |
| | CACAGA | | AUCACC | | |

TABLE 4b
Modified sequences of dsRNA targeting human PRNP mRNA

| | | | | | Start |
|---|---|---|---|---|---|
| | | | | | position of |
| | | | | | antisense |
| | | | | | strand target |
| | | | | | region of |
| | | | | | human PRNP |
| | | SEQ | | SEQ | transcript |
| dsRNA | Sense Strand | ID | Antisense Strand | ID | NM_000311.5 |
| No. | (5′ to 3′) | NO | (5′ to 3′) | NO | (SEQ ID NO: 61) |
| 2 | mC*mA*mCmGmAmCm | 43 | mU*fG*mUmGmAmUmAmU | 44 | 594 |
| | UmGfCfGfUmCmAmAm | | mUmGmAmCmGfCmAfGmU | | |
| | UmAmUmCmA*mC*mA | | mCmGmUmG*mC*mA | | |
| 11 | mA*mU*mUmAmCmUm | 45 | mU*fU*mAmAmCmAmUmU | 46 | 2154 |
| | UmUfUfCfUmGmCmAm | | mGmCmAmGmAfAmAfAmG | | |
| | AmUmGmUmU*mA*mA | | mUmAmAmU*mA*mC | | |
| 12 | mU*mA*mCmAmAmUm | 47 | mU*fA*mCmGmAmUmUmC | 48 | 2358 |
| | GmUfGfCfAmCmUmGm | | mAmGmUmGmCfAmCfAmU | | |
| | AmAmUmCmG*mU*mA | | mUmGmUmA*mA*mG | | |
| 13 | mG*mA*mAmGmUmGm | 49 | mU*fA*mGmAmAmUmUmU | 50 | 2070 |
| | GmAfAfAfAmAmGmAm | | mCmUmUmUmUfUmCfCmA | | |
| | AmAmUmUmC*mU*mA | | mCmUmUmC*mA*mA | | |
| 14 | mC*mA*mCmCmAmUm | 51 | mU*fA*mAmAmAmGmAmU | 52 | 952 |
| | CmUfUfUfCmUmAmAm | | mUmAmGmAmAfAmGfAmU | | |
| | UmCmUmUmU*mU*mA | | mGmGmUmG*mA*mA | | |
| 15 | mC*mU*mAmAmUmGm | 53 | mU*fU*mAmCmAmAmAmA | 54 | 2287 |
| | CmAfUfUfAmAmCmUm | | mGmUmUmAmAfUmGfCmA | | |
| | UmUmUmGmU*mA*mA | | mUmUmAmG*mA*mC | | |
| 16 | mG*mC*mUmCmAmGm | 55 | mU*fA*mGmGmGmCmAmU | 56 | 1306 |
| | UmAfUfAfCmUmAmAm | | mUmAmGmUmAfUmAfCmU | | |
| | UmGmCmCmC*mU*mA | | mGmAmGmC*mU*mC | | |
| 17 | mU*mU*mCmCmCmUm | 57 | mU*fA*mUmAmUmCmAmA | 58 | 2115 |
| | GmAfAfUfUmGmUmUm | | mAmCmAmAmUfUmCfAmG | | |
| | UmGmAmUmA*mU*mA | | mGmGmAmA*mU*mA | | |
| 18 | mU*mG*mAmUmGmUm | 59 | mU*fC*mUmGmUmGmAmA | 60 | 1511 |
| | UmUfUfAfCmUmUmUm | | mAmAmGmUmAfAmAfAmC | | |
| | UmCmAmCmA*mG*mA | | mAmUmCmA*mC*mC | | |

Abbreviations - “m” indicates 2′-OMe; “f” indicates 2′-fluoro; “*” indicates phosphorothioate linkage; unless otherwise noted, the 5′ position of the AS can include 5′-phosphate or 5′- vinylphosphonate (VP).

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl (e.g., 2′-O—C₁₆ alkyl).

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, at least one nucleotide of the sense strand is an unmodified RNA nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, at least one nucleotide of the sense strand is 2′ deoxy nucleotide (DNA).

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 5.

TABLE 5
A basic or inverted abasic (iAb) moieties

| | Structure |
|---|---|
| 1 (abasic) | 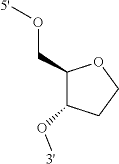 |
| 2 (iAb) | 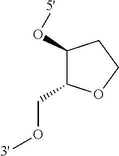 |

″5′′′ and ″3′′′ indicate 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human PRNP mRNA are provided in Table 4b.

In some embodiments, the sense strand comprises SEQ ID NO: 43, and the antisense strand comprises SEQ ID NO: 44. In some embodiments, the sense strand comprises SEQ ID NO: 45, and the antisense strand comprises SEQ ID NO: 46. In some embodiments, the sense strand comprises SEQ ID NO: 47, and the antisense strand comprises SEQ ID NO: 48. In some embodiments, the sense strand comprises SEQ ID NO: 49, and the antisense strand comprises SEQ ID NO: 50. In some embodiments, the sense strand comprises SEQ ID NO: 51, and the antisense strand comprises SEQ ID NO: 52. In some embodiments, the sense strand comprises SEQ ID NO: 53, and the antisense strand comprises SEQ ID NO: 54. In some embodiments, the sense strand comprises SEQ ID NO: 55, and the antisense strand comprises SEQ ID NO: 56. In some embodiments, the sense strand comprises SEQ ID NO: 57, and the antisense strand comprises SEQ ID NO: 58. In some embodiments, the sense strand comprises SEQ ID NO: 59, and the antisense strand comprises SEQ ID NO: 60.

In some embodiments, the sense strand consists of SEQ ID NO: 43, and the antisense strand consists of SEQ ID NO: 44. In some embodiments, the sense strand consists of SEQ ID NO: 45, and the antisense strand consists of SEQ ID NO: 46. In some embodiments, the sense strand consists of SEQ ID NO: 47, and the antisense strand consists of SEQ ID NO: 48. In some embodiments, the sense strand consists of SEQ ID NO: 49, and the antisense strand consists of SEQ ID NO: 50. In some embodiments, the sense strand consists of SEQ ID NO: 51, and the antisense strand consists of SEQ ID NO: 52. In some embodiments, the sense strand consists of SEQ ID NO: 53, and the antisense strand consists of SEQ ID NO: 54. In some embodiments, the sense strand consists of SEQ ID NO: 55, and the antisense strand consists of SEQ ID NO: 56. In some embodiments, the sense strand consists of SEQ ID NO: 57, and the antisense strand consists of SEQ ID NO: 58. In some embodiments, the sense strand consists of SEQ ID NO: 59, and the antisense strand consists of SEQ ID NO: 60.

In some embodiments, the dsRNA comprises a sense strand that comprises a sequence that has 1, 2, or 3 differences from a sense stand sequence in Table 4a or 4b. In some embodiments, the dsRNA comprises an antisense strand that comprises a sequence that has 1, 2, or 3 differences from an antisense stand sequence in Table 4a or 4b.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP—IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

The RNAi agent described herein can be made by a variety of procedures known to one of ordinary skill in the art, some of which are illustrated in the preparations and examples below, e.g., in Examples 1-3. One of ordinary skill in the art recognizes that the specific synthetic steps for each of the routes described may be combined in different ways, or in conjunction with steps from different schemes, to prepare the RNAi agent. The product of each step can be recovered by conventional methods well known in the art, including extraction, evaporation, precipitation, chromatography, filtration, trituration, and crystallization. The reagents and starting materials are readily available to one of ordinary skill in the art.

In some embodiments, the TfR binding protein with native or engineered cysteines described herein can be first treated with a reducing agent, e.g., DTT, and then re-oxidized with an oxidizing agent, e.g., DHAA. The resulting oxidized TfR binding protein is then incubated with a linker functionalized dsRNA, e.g., linker-dsRNA, to produce the conjugated RNAi agent.

### Pharmaceutical Composition

In another aspect, provided herein are pharmaceutical compositions comprising any of the PRNP RNAi agents described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating a prion disease in a patient in need thereof, and such method comprises administering to the patient an effective amount of the PRNP RNAi agent or a pharmaceutical composition described herein. Exemplary prion disease includes, but are not limited to, Creutzfeldt-Jakob disease, including familial and sporadic Creutzfeldt-Jakob disease, fatal familial insomnia, Gerstmann-Straussler disease, Huntington disease-like 1, kuru.

The PRNP RNAi agent or a pharmaceutical composition comprising PRNP RNAi agent can be administered to the patient intravenously or subcutaneously.

PRNP RNAi agent dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are PRNP RNAi agents or pharmaceutical compositions comprising a PRNP RNAi agent for use in reducing PRNP expression. Also provided herein are PRNP RNAi agents or the pharmaceutical composition comprising a PRNP RNAi agent for use in a therapy. Also provided herein are PRNP RNAi agents or pharmaceutical compositions comprising a PRNP RNAi agent for use in the treatment of a prion disease. Also provided herein are uses of PRNP RNAi agents in the manufacture of a medicament for the treatment of a prion disease.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); A1-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “antigen binding domain”, as used herein, refers to a portion of an antibody or antibody fragment that binds an antigen or an epitope of the antigen. For example, “TfR binding domain” refers to a portion of an antibody or antibody fragment that binds TfR or an epitope of TfR.

The term “heterodimeric antibody”, as used herein, refers to an antibody that comprises two distinct antigen-binding domains.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 5.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, a “null arm” means an antibody arm that does not bind any known human target.

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ end of an oligonucleotide in place of a 5′-phosphate, which is sometimes susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

As used herein, “PRNP” (also known as PrP; ASCR; KURU; PRIP; CD230; CJD; GSS) refers to a human PRNP mRNA transcript or a human PRNP protein. The nucleotide sequence of the longest human PRNP mRNA transcript can be found at NM_000311.5:

(SEQ ID NO: 61)

| | |
|---|---|
| 1 | GCCAGTCGCT GACAGCCGCG GCGCCGCGAG CTTCTCCTCT CCTCACGACC GAGGCAGAGC |
| 61 | AGTCATTATG GCGAACCTTG GCTGCTGGAT GCTGGTTCTC TTTGTGGCCA CATGGAGTGA |
| 121 | CCTGGGCCTC TGCAAGAAGC GCCCGAAGCC TGGAGGATGG AACACTGGGG GCAGCCGATA |
| 181 | CCCGGGGCAG GGCAGCCCTG GAGGCAACCG CTACCCACCT CAGGGCGGTG GTGGCTGGGG |
| 241 | GCAGCCTCAT GGTGGTGGCT GGGGGCAGCC TCATGGTGGT GGCTGGGGGC AGCCCCATGG |
| 301 | TGGTGGCTGG GGACAGCCTC ATGGTGGTGG CTGGGGTCAA GGAGGTGGCA CCCACAGTCA |
| 361 | GTGGAACAAG CCGAGTAAGC CAAAAACCAA CATGAAGCAC ATGGCTGGTG CTGCAGCAGC |
| 421 | TGGGGCAGTG GTGGGGGGCC TTGGCGGCTA CATGCTGGGA AGTGCCATGA GCAGGCCCAT |
| 481 | CATACATTTC GGCAGTGACT ATGAGGACCG TTACTATCGT GAAAACATGC ACCGTTACCC |
| 541 | CAACCAAGTG TACTACAGGC CCATGGATGA GTACAGCAAC CAGAACAACT TTGTGCACGA |
| 601 | CTGCGTCAAT ATCACAATCA AGCAGCACAC GGTCACCACA ACCACCAAGG GGGAGAACTT |
| 661 | CACCGAGACC GACGTTAAGA TGATGGAGCG CGTGGTTGAG CAGATGTGTA TCACCCAGTA |
| 721 | CGAGAGGGAA TCTCAGGCCT ATTACCAGAG AGGATCGAGC ATGGTCCTCT TCTCCTCTCC |
| 781 | ACCTGTGATC CTCCTGATCT CTTTCCTCAT CTTCCTGATA GTGGGATGAG GAAGGTCTTC |
| 841 | CTGTTTTCAC CATCTTTCTA ATCTTTTTCC AGCTTGAGGG AGGCGGTATC CACCTGCAGC |
| 901 | CCTTTTAGTG GTGGTGTCTC ACTCTTTCTT CTCTCTTTGT CCCGGATAGG CTAATCAATA |
| 961 | CCCTTGGCAC TGATGGGCAC TGGAAAACAT AGAGTAGACC TGAGATGCTG GTCAAGCCCC |
| 1021 | CTTTGATTGA GTTCATCATG AGCCGTTGCT AATGCCAGGC CAGTAAAAGT ATAACAGCAA |
| 1081 | ATAACCATTG GTTAATCTGG ACTTATTTTT GGACTTAGTG CAACAGGTTG AGGCTAAAAC |
| 1141 | AAATCTCAGA ACAGTCTGAA ATACCTTTGC CTGGATACCT CTGGCTCCTT CAGCAGCTAG |
| 1201 | AGCTCAGTAT ACTAATGCCC TATCTTAGTA GAGATTTCAT AGCTATTTAG AGATATTTTC |
| 1261 | CATTTTAAGA AAACCCGACA ACATTTCTGC CAGGTTTGTT AGGAGGCCAC ATGATACTTA |
| 1321 | TTCAAAAAAA TCCTAGAGAT TCTTAGCTCT TGGGATGCAG GCTCAGCCCG CTGGAGCATG |
| 1381 | AGCTCTGTGT GTACCGAGAA CTGGGGTGAT GTTTTACTTT TCACAGTATG GGCTACACAG |
| 1441 | CAGCTGTTCA ACAAGAGTAA ATATTGTCAC AACACTGAAC CTCTGGCTAG AGGACATATT |
| 1501 | CACAGTGAAC ATAACTGTAA CATATATGAA AGGCTTCTGG GACTTGAAAT CAAATGTTTG |
| 1561 | GGAATGGTGC CCTTGGAGGC AACCTCCCAT TTTAGATGTT TAAAGGACCC TATATGTGGC |
| 1621 | ATTCCTTTCT TTAAACTATA GGTAATTAAG GCAGCTGAAA AGTAAATTGC CTTCTAGACA |
| 1681 | CTGAAGGCAA ATCTCCTTTG TCCATTTACC TGGAAACCAG AATGATTTTG ACATACAGGA |
| 1741 | GAGCTGCAGT TGTGAAAGCA CCATCATCAT AGAGGATGAT GTAATTAAAA AATGGTCAGT |
| 1801 | GTGCAAAGAA AAGAACTGCT TGCATTTCTT TATTTCTGTC TCATAATTGT CAAAAACCAG |
| 1861 | AATTAGGTCA AGTTCATAGT TTCTGTAATT GGCTTTTGAA TCAAAGAATA GGGAGACAAT |
| 1921 | CTAAAAAATA TCTTAGGTTG GAGATGACAG AAATATGATT GATTTGAAGT GGAAAAAGAA |
| 1981 | ATTCTGTTAA TGTTAATTAA AGTAAAATTA TTCCCTGAAT TGTTTGATAT TGTCACCTAG |
| 2041 | CAGATATGTA TTACTTTTCT GCAATGTTAT TATTGGCTTG CACTTTGTGA GTATTCTATG |
| 2101 | TAAAAATATA TATGTATATA AAATATATAT TGCATAGGAC AGACTTAGGA GTTTTGTTTA |
| 2161 | GAGCAGTTAA CATCTGAAGT GTCTAATGCA TTAACTTTTG TAAGGTACTG AATACTTAAT |
| 2221 | ATGTGGGAAA CCCTTTTGCG TGGTCCTTAG GCTTACAATG TGCACTGAAT CGTTTCATGT |
| 2281 | AAGAATCCAA AGTGGACACC ATTAACAGGT CTTTGAAATA TGCATGTACT TTATATTTTC |
| 2341 | TATATTTGTA ACTTTGCATG TTCTTGTTTT GTTATATAAA AAAATTGTAA ATGTTTAATA |
| 2401 | TCTGACTGAA ATTAAACGAG CGAAGATGAG CACCA. |

The corresponding amino acid sequence of human PRNP protein can be found at NP_000302.1:

```
(SEQ ID NO: 62)
    1 MANLGCWMLV LFVATWSDLG LCKKRPKPGG WNTGGSRYPG QGSPGGNRYP PQGGGGWGQP
  61 HGGGWGQPHG GGWGQPHGGG WGQPHGGGWG QGGGTHSQWN KPSKPKTNMK HMAGAAAAGA
121 VVGGLGGYML GSAMSRPIIH FGSDYEDRYY RENMHRYPNQ VYYRPMDEYS NQNNFVHDCV
181 NITIKQHTVT TTTKGENFTE TDVKMMERVV EQMCITQYER ESQAYYQRGS SMVLFSSPPV
241 ILLISFLIFL IVG.
```

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “polypeptide” or “protein”, as used herein, refers to a polymer of amino acid residues. The term applies to polymers comprising naturally occurring amino acids and polymers comprising one or more non-naturally occurring amino acids.

As used herein, “RNAi,” “RNAi agent,” “iRNA,” “iRNA agent,” or “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex (e.g., a double stranded RNA).

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The following examples are offered to illustrate, but not to limit, the claimed inventions.

### Examples

### Example 1: Generation and Characterization of TfR Binding Proteins

### Generation of Human TfR Binding Proteins

Antibody against human TfR was generated by immunizing AlivaMab® transgenic mice with the extracellular domains of human Transferrin Receptor 1 protein with a His tag (hTfR-ECD-6His, SEQ ID NO: 64, see Table 6) and mouse Transferrin Receptor protein with a His tag (mTfR-ECD-6His, SEQ ID NO:63). Antigen positive B-cells were sorted from pooled spleens. Binding of individual antibodies cloned from those B-cells to his-tagged hTfR-ECD was verified.

Additional antibody against human TfR was generated by immunizing AlivaMab® transgenic mice with the apical domain of human Transferrin Receptor 1 protein with a His tag (hTfR-ApD-6His, SEQ ID NO: 65, see Table 6). Antigen positive B-cells were sorted from pooled spleens. Binding of individual antibodies cloned from those B-cells to his-tagged hTfR-ECD was verified.

TABLE 6
Sequences of the immunogens used to generate human or mouse TfR antibodies.

| Immunogen | Sequence | SEQ ID NO |
|---|---|---|
| mTfR-ECD-6His | HHHHHHCKRVEQKEECVKLAETEETDKSETMETEDV | 63 |
| | PTSSRLYWADLKTLLSEKLNSIEFADTIKQLSQNTYTP | |
| | REAGSQKDESLAYYIENQFHEFKFSKVWRDEHYVKI | |
| | QVKSSIGQNMVTIVQSNGNLDPVESPEGYVAFSKPTE | |
| | VSGKLVHANFGTKKDFEELSYSVNGSLVIVRAGEITF | |
| | AEKVANAQSFNAIGVLIYMDKNKFPVVEADLALFGH | |
| | AHLGTGDPYTPGFPSFNHTQFPPSQSSGLPNIPVQTISR | |
| | AAAEKLFGKMEGSCPARWNIDSSCKLELSQNQNVKL | |
| | IVKNVLKERRILNIFGVIKGYEEPDRYVVVGAQRDAL | |
| | GAGVAAKSSVGTGLLLKLAQVFSDMISKDGFRPSRSII | |
| | FASWTAGDFGAVGATEWLEGYLSSLHLKAFTYINLD | |
| | KVVLGTSNFKVSASPLLYTLMGKIMQDVKHPVDGKS | |
| | LYRDSNWISKVEKLSFDNAAYPFLAYSGIPAVSFCFCE | |
| | DADYPYLGTRLDTYEALTQKVPQLNQMVRTAAEVA | |
| | GQLIIKLTHDVELNLDYEMYNSKLLSFMKDLNQFKT | |
| | DIRDMGLSLQWLYSARGDYFRATSRLTTDFHNAEKT | |
| | NRFVMREINDRIMKVEYHFLSPYVSPRESPFRHIFWGS | |
| | GSHTLSALVENLKLRQKNITAFNETLFRNQLALATWT | |
| | IQGVANALSGDIWNIDNEF | |
| hTfR-ECD-6His | HHHHHHCKGVEPKTECERLAGTESPVREEPGEDFPA | 64 |
| | ARRLYWDDLKRKLSEKLDSTDFTGTIKLLNENSYVPR | |
| | EAGSQKDENLALYVENQFREFKLSKVWRDQHFVKIQ | |
| | VKDSAQNSVIIVDKNGRLVYLVENPGGYVAYSKAAT | |
| | VTGKLVHANFGTKKDFEDLYTPVNGSIVIVRAGKITF | |
| | AEKVANAESLNAIGVLIYMDQTKFPIVNAELSFFGHA | |
| | HLGTGDPYTPGFPSFNHTQFPPSRSSGLPNIPVQTISRA | |
| | AAEKLFGNMEGDCPSDWKTDSTCRMVTSESKNVKL | |
| | TVSNVLKEIKILNIFGVIKGFVEPDHYVVVGAQRDAW | |
| | GPGAAKSGVGTALLLKLAQMFSDMVLKDGFQPSRSII | |
| | FASWSAGDFGSVGATEWLEGYLSSLHLKAFTYINLD | |
| | KAVLGTSNFKVSASPLLYTLIEKTMQNVKHPVTGQFL | |
| | YQDSNWASKVEKLTLDNAAFPFLAYSGIPAVSFCFCE | |
| | DTDYPYLGTTMDTYKELIERIPELNKVARAAAEVAG | |
| | QFVIKLTHDVELNLDYERYNSQLLSFVRDLNQYRADI | |
| | KEMGLSLQWLYSARGDFFRATSRLTTDFGNAEKTDR | |
| | FVMKKLNDRVMRVEYHFLSPYVSPKESPFRHVFWGS | |
| | GSHTLPALLENLKLRKQNNGAFNETLFRNQLALATW | |
| | TIQGAANALSGDVWDIDNEF | |
| hTfR-ApD-6His | HHHHHHHHGKPIPNPLLGLDSTGGGGSDSAQNSVIIV | 65 |
| | DKNGRLVYLVENPGGYVAYSKAATVTGKLVHANFG | |
| | TKKDFEDLYTPVNGSIVIVRAGKITFAEKVANAESLN | |
| | AIGVLIYMDQTKFPIVNAELSFFGHAHLGGGGGGLPN | |
| | IPVQTISRAAAEKLFGNMEGDCPSDWKTDSTCRMVTS | |
| | ESKNVKLTVS | |

Affinity variants of the generated human TfR antibodies were made by systematically introducing mutations into individual CDR of each antibody and the resulting variants were subjected to multiple rounds of selection with decreasing concentrations of antigen and/or increasing periods of dissociation to isolate clones with improved affinities. The sequences of individual variants were used to construct a combinatorial library which was subjected to an additional round of selection with increased stringency to identify additive or synergistic mutational pairings between the individual CDR regions. Individual combinatorial clones are sequenced. The heavy chain and light chain CDRs and VH/VL sequences of the human TfR binding domains and proteins are provided in Table 1a.

Human TfR binding proteins were generated by recombinant DNA technology. Such human TfR binding proteins can be expressed in a mammalian cell line such as HEK293 or CHO, either transiently or stably transfected with an expression system using an optimal predetermined HC:LC vector ratio or a single vector system encoding both HC and LC. Clarified media, into which the protein has been secreted, can be purified using the commonly used techniques.

### Binding Affinity

Binding affinity and binding stoichiometry of some exemplified human TfR binding proteins to human and cynomolgus TfR was characterized using a surface plasmon resonance assay on a Biacore 8K instrument primed with HBS-EP+ (10 mM Hepes pH7.4+150 mM NaCl+3 mM EDTA+0.05% (w/v) surfactant P20) running buffer and analysis temperature set at 37° C. Target human and cynomologus TfR ECD's were immobilized on a CM4 chip (Cytiva P/N 29104989) using standard NHS-EDC amine coupling. The TfR binding proteins were prepared at a final concentration of 0.3, 0.1, 0.033, 0.01, 0.0033, 0.001, 0.00033, 0.0001 UM respectively by dilution of stock solution into running buffer.

Binding analysis was performed in a multi-cycle kinetics manner. Each analysis cycle consists of (1) injection of the lowest to highest concentration proteins over all Fc at 50 μL/min for 140 seconds followed by return to buffer flow for 400 seconds to monitor dissociation phase; (2) regeneration of chip surfaces with injection of 3M magnesium chloride, for 30 seconds at 100 μL/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 7a.

TABLE 7a
Binding Affinity of Exemplified human TfR binding
proteins to human or cynomolgus TfR at 37° C.

| | | Standard error of | | Standard error of |
|---|---|---|---|---|
| Human TfR | Human TfR K_{D} | the mean, Human TfR | Cyno TfR K_{D} | the mean, Cyno TfR |
| binding | (Biacore, nM) | K_{D} (Biacore, nM) | (Biacore, nM) | K_{D} (Biacore, nM) |
| proteins (TBP) | at 37° C. | n = 3 | at 37° C. | n = 3 |
| TBP3 | 32.087 | 11.795 | 66.565 | 11.695 |
| TBP4 | 153.642 | 7.949 | 300.180 | 2.565 |
| TBP5 | 0.522 | 0.284 | 502.210 | 8.129 |

Binding affinity of some exemplified human TfR binding proteins to human TfR was characterized using a surface plasmon resonance assay on a Biacore 8K instrument primed with HBS-EP+ (10 mM Hepes pH7.6+150 mM NaCl+3 mM EDTA+0.05% (w/v) surfactant P20) running buffer and analysis temperature set at 25° C. Target human TfR ECDs were immobilized on a CM3 chip (Cytiva P/N BR100536) using standard NHS-EDC amine coupling. The TfR binding proteins were prepared at a final concentration of 1.8, 0.6, 0.2, 0.067, 0.022, 0.0074, 0.0025 μM respectively by dilution of stock solution into running buffer.

Binding analysis was performed in a single-cycle or multi-cycle kinetics manner. Each analysis cycle consisted of (1) injection of the lowest to highest concentration proteins over all flow channel at 50 μL/min for 200 seconds followed by return to buffer flow for 900 seconds to monitor dissociation phase; (2) regeneration of chip surfaces with injection of 3M magnesium chloride, for 30 seconds at 100 L/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 7b.

TABLE 7b
Binding Affinity of Exemplified human TfR binding
proteins to human or cynomolgus TfR at 25° C.

| TBP | Ka (1/Ms) | Kd (1/s) | KD (M) |
|---|---|---|---|
| TBP5 | 6.46E5 | 7.99E−3 | 1.24E−8 |
| TBP8 | 3.61E5 | 6.71E−3 | 1.86E−8 |

### Example 2: Synthesis and Characterization of dsRNAs Targeting PRNP

Single strands (sense and antisense) of the dsRNA duplexes were typically synthesized on solid support via a K&A H-8 (K&A Labs GmbH) or a similar automated oligonucleotide synthesizer. The sequences of the sense and antisense strands were shown in Table 4a or 4b.

Standard reagents were used in the oligo synthesis (Table 8), where 0.1M [(dimethylaminomethylene)amino]-3H-1,2,4-dithiazole-5-thione (DDTT) in pyridine was used as the sulfurization reagent. All monomers (Table 9) except for OMe U (0.1M in 20% DMF/ACN) were made at 0.1M in ACN and contained a molecular sieves trap bag.

The oligonucleotides were cleaved and deprotected (C/D) using AMA (1:1 mixture of concentrated ammonia and 40% wt methylamine in water) at room temperature for 2 hours. C/D was determined complete by IP-RP LCMS when the resulting mass data confirmed the identity of sequence. Dependent on scale, the CPG was filtered via Acrodisc 32 mm syringe filter with 0.2 μm Supor membrane. The CPG was back washed/rinsed with 50% EtOH/RNAse free water then filtered through the same filtering device and combined with the first filtrate. This was repeated twice. The material was then divided evenly into 50 mL falcon tubes to remove organics via Genevac™.

The crude oligonucleotides were purified via AKTA™ Pure purification system using anion-exchange (AEX). A Cytiva C 16/40 Column manually packed with Capto™ Q ImpRes (13 cm bed height) was used at room temperature with MPA: 20 mM sodium phosphate buffer, 20% ACN, pH 7.0 and MPB: 20 mM sodium phosphate buffer, 1.5M NaBr, 20% ACN, pH 7.0. In all cases, fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, approximately 1 −2 mL of sample was recovered and transferred to a 5 mL Eppendorf tube. The final desalted oligonucleotides were analyzed for concentration (nano drop at A260), characterized by IP-RP LCMS for mass purity and UV-purity.

For the preparation of duplexes, equimolar amounts of sense and antisense strand were combined, added with 10×PBS to a final PBS concentration of 1×, and heated at 85° C. for 6 minutes then slowly cooled to 6° C. with a cooling rate of 0.5° C./min. Integrity of the duplex was confirmed by LCMS using IP-RP. For in vivo analysis, the appropriate amount of duplex was either lyophilized or diluted with 1×PBS for rodent studies and a CSF for non-human primate studies.

TABLE 8
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
[(Dimethylaminomethylene)amino]-3H-1,2,4-dithiazole-5-
thione (DDTT) (0.1M in Pyridine)
Diethylamine (20% in Acetonitrile)

TABLE 9
Phosphoramidites

| Phosphoramidite | Abbreviation | Supplier | Catalog # | CAS |
|---|---|---|---|---|
| DMT-2′-F—A(Bz)- | fA | Chemgenes | ANP-9151 | 136834-22-5 |
| CE | | | | |
| Phosphoamidite | | | | |
| DMT-2′- | fC | Chemgenes | ANP-9152 | 159414-99-0 |
| F—C(Ac)—CE | | | | |
| Phosphoamidite | | | | |
| DMT-2′- | fG | Chemgenes | ANP-9158 | 159414-99-0 |
| F—G(Ac)—CE | | | | |
| Phosphoamidite | | | | |
| DMT-2′-F—U—CE | fU | Chemgenes | ANP-9154 | 146954-75-8 |
| Phosphoamidite | | | | |
| DMT-2′- | mA | Chemgenes | ANP-5751 | 110782-31-5 |
| O—Me—A(Bz)-CE | | | | |
| Phosphoamidite | | | | |
| DMT-2′- | mC | Chemgenes | ANP-6756 | 199593-09-4 |
| O—Me—C(Ac)—CE | | | | |
| Phosphoamidite | | | | |
| DMT-2′- | mG | Chemgenes | ANP-5763 | 150780-67-9 |
| O—Me—G(Ac)—CE | | | | |
| Phosphoamidite | | | | |
| DMT-2′- | mU | Chemgenes | ANP-5754 | 110764-79-9 |
| O—Me—U—CE | | | | |
| Phosphoamidite | | | | |
| 5′bis(POM) vinyl | POM-VPmU | Hongene | PR5-032 | BVPMUP23B2A1 |
| phosphate-2′- | | | | |
| Ome-U3′CE | | | | |
| phosphoroamidite | | | | |

### Example 3: Generation of PRNP RNAi agents

Certain abbreviations are defined as follows: “ACN” refers to acetonitrile; “aAEX” refers to analytical anion exchange; “AS” refers to antisense strand; “DAR” refers to drug/siRNA to antibody/protein ratio; “DCM” refers to dichloromethane; “DHAA” refers to dehydroascorbic acid; “dsRNA” refers to double stranded ribonucleic acid; “DTT” refers to dithiothreitol; “h” refers to hours; “HPLC” refers to high-performance liquid chromatography; “LC/MS” refers to liquid chromatography mass spectrometry; “LTQ/MS” refers to linear ion trap mass spectrometer; “min” refers to minutes; “MSPT” refers to 4-(5-methylsulfonyl-1H-tetrazole-1yl)phenol; “MW” refers to molecular weight; “MWCO” refers to molecular weight cut-off; “NHS” refers to N-hydroxysuccinimide; “OD” refers to 4-(5-(methylsulfonyl)-1,3,4-oxadiazol-2-yl)phenol; “PBS” phosphate-buffered saline; “PEG” refers to polyethylene glycol; “RNAi” refers to RNA interference; “rpm” refers to revolutions per minute; “SEC” refers to size exclusion chromatography; “siRNA” refers to small interfering RNA; “SMCC” refers to succinimidyl-4-(N-maleimidomethyl)cyclohexane-1-carboxylate; “SS” refers to sense strand; “TCO” refers to trans-cyclo-octene; “TfR” refers to transferrin receptor; “THF” refers to tetrahydrofuran; “TRIS” refers to tris(hydroxymethyl) aminomethane; “UPLC” refers to ultra performance liquid chromatography; and “UV” refers to ultraviolet.

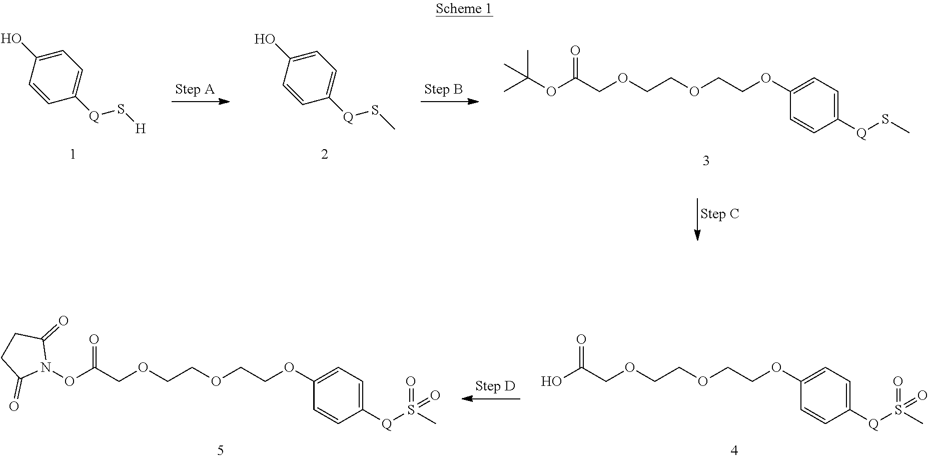

Q is 1,3,4-oxadiazole or 1H-tetrazole.

Scheme 1 depicts the synthetic route to the intermediates used to form the final MSPT and OD linkers shown in Table 3.

Scheme 1, step A depicts the methylation of the thiol on compound (1) using iodomethane and a suitable base such as DIEA in a solvent such as THF to give compound (2). Step B shows an alkylation of compound (2) with tert-butyl 2-(2-(2-bromoethoxy)ethoxy)acetate using a base such as potassium carbonate in a solvent such as acetone to give compound (3). Step C shows the oxidation of compound (3) with hydrogen peroxide and ammonium molybdate (VI) tetrahydrate in a solvent such as EtOH followed by an acidic deprotection using an acid such as TFA in a solvent such as DCM to give compound (4). Note that in the case of the 1H-tetrazole, the deprotection took place during the oxidation step. Step D depicts a coupling of compound (4) and 1-hydroxypyrrolidine-2,5-dione using EDCI in a solvent system such as DCM and THE to give compound (5).

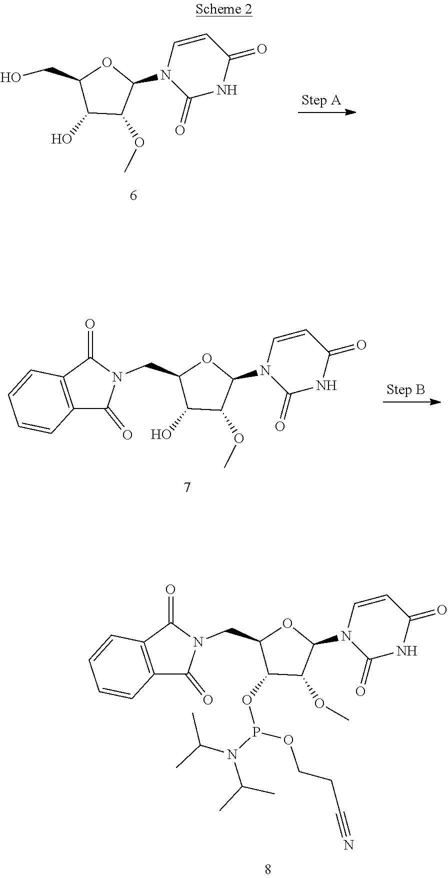 [SMILES: CO[C@@H]1[C@H](O)[C@@H](CO)O[C@H]1n1ccc(=O)[nH]c1=O ; CO[C@@H]1[C@H](O)[C@@H](CN2C(=O)c3ccccc3C2=O)O[C@H]1n1ccc(=O)[nH]c1=O ; CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

Scheme 2, step A shows the coupling of compound (6) and isoindoline-1,3-dione using DIAD and tributyl phosphine in a solvent such as THE to give compound (7). Step B depicts the phosphorylation of compound (7) with 2-cyanoethyl-N,N-diisopropylchlorophosphoramidite using a base such as DIEA in a solvent such as DCM to give compound (8).

### Preparation 1

### 4-(5-(Methylthio)-1,3,4-oxadiazol-2-yl)phenol

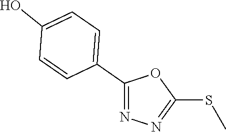

A solution of 4-(5-mercapto-1,3,4-oxadiazol-2-yl)phenol (3.00 g, 15.4 mmol) in THF (50 mL) was cooled to 0° C. DIEA (3.46 mL, 20.1 mmol) was added then stirred for 5 minutes before adding iodomethane (2.85 g, 20.1 mmol) dropwise over a period of 1 minute. The mixture was stirred at 0° C. for 5 minutes, and then stirred at ambient temperature for 2 hours. After this time, the mixture was diluted with DCM (100 mL) and washed with saturated aqueous NH₄Cl (pH was adjusted to ˜5 by adding citric acid solution, 2×50 mL). The organic layer was separated, dried over sodium sulfate, and concentrated in vacuo to give the title compound as a pale-yellow solid (520 mg, 97%). ES/MS m/z: 209 (M+H).

The compound in Table 10 was prepared in a manner essentially analogous to that found in Preparation 1.

TABLE 10

| | | | ES/MS |
|---|---|---|---|
| Prep | Name | Structure | m/z |
| 2 | 4-(5-(Methylthio)-1H- tetrazol-1-yl)phenol | 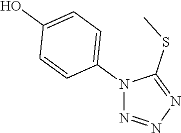 | 209 (M + H) |

### Preparation 3

### tert-Butyl 2-(2-(2-(4-(5-(methylthio)-1,3,4-oxadiazol-2-yl)phenoxy)ethoxy)ethoxy)acetate

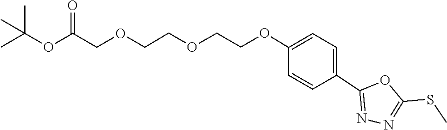 [SMILES: CSc1nnc(-c2ccc(OCCOCCOCC(=O)OC(C)(C)C)cc2)o1]

In a pressure vessel, tert-butyl 2-(2-(2-bromoethoxy)ethoxy)acetate (5.5 g, 20 mmol) and potassium carbonate (4.2 g, 30 mmol) were added to 4-(5-(methylthio)-1,3,4-oxadiazol-2-yl)phenol (3.3 g, 15 mmol) in acetone (60 mL). The pressure vessel was sealed and heated at 70° C. for 5 hours with vigorous stirring. After this time, the mixture was cooled to ambient temperature. The mixture was filtered while washing through with EtOAc/DCM. The filtrate was concentrated in vacuo and purified via silica gel column chromatography eluting with 0-100% EtOAc/DCM to give the title compound as a white solid (4.8 g, 74%). ES/MS m/z: 411 (M+H).

The compound in Table 11 was prepared in a manner essentially analogous to that found in Preparation 3.

TABLE 11

| | | | ES/MS |
|---|---|---|---|
| Prep | Name | Structure | m/z |
| 4 | tert-Butyl 2-(2-(2-(4-(5- (methylthio)-1H-tetrazol-1- yl)phenoxy)ethoxy) ethoxy)acetate | 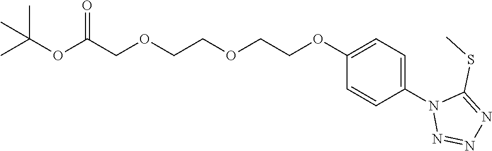 [SMILES: CSc1nnnn1-c1ccc(OCCOCCOCC(=O)OC(C)(C)C)cc1] | 411 (M + H) |

### Preparation 5

### tert-Butyl 2-(2-(2-(4-(5-(methylsulfonyl)-1,3,4-oxadiazol-2-yl)phenoxy)ethoxy)ethoxy)acetate

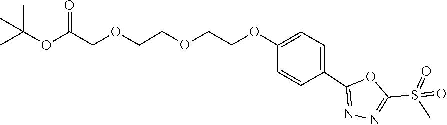 [SMILES: CC(C)(C)OC(=O)COCCOCCOc1ccc(-c2nnc(S(C)(=O)=O)o2)cc1]

tert-Butyl 2-(2-(2-(4-(5-(methylthio)-1,3,4-oxadiazol-2-yl)phenoxy)ethoxy)ethoxy)acetate (5.20 g,12.7 mmol) was dissolved in EtOH (100 mL) and cooled to 5-10° C. Then, 30% hydrogen peroxide (10 mL, 97 mmol) was added, followed by ammonium molybdate (VI) tetrahydrate (501 mg, 0.405 mmol). After two hours of vigorous stirring, additional 30% hydrogen peroxide (15 mL, 145.5 mmol) and ammonium molybdate (VI) tetrahydrate (1 g, 0.910 mmol) were added. The mixture was stirred for 6 hours, then diluted with DCM (150 mL) and washed with saturated aqueous sodium chloride solution. The organic phase was separated, dried over sodium sulfate, and concentrated in vacuo. The resulting residue was triturated with MeOH to provide the first lot of the title compound. The solvent from the mother liquor was concentrated in vacuo and purified via silica gel column chromatography eluting with 0-100% EtOAc/DCM to give additional product as white solid. The recovered materials were combined to give the title compound as a white solid (5.3 g, 90%).

ES/MS m/z: 387 (M+H-tBu).

The compound in Table 12 was prepared in a manner essentially analogous to that found in Preparation 5.

TABLE 12

| | | | ES/MS |
|---|---|---|---|
| Prep | Name | Structure | m/z |
| 6 | 2-(2-(2-(4-(5- (methylsulfonyl)- 1H-tetrazol-1- yl)phenoxy)ethoxy) ethoxy)acetic acid | 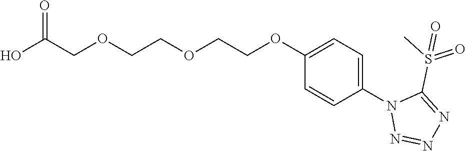 [SMILES: CS(=O)(=O)c1nnnn1-c1ccc(OCCOCCOCC(=O)O)cc1] | 385 (M − H) |

Note
that the conditions were analogous, but the t-butyl group was removed in the process.

Note that the conditions were analogous, but the t-butyl group was removed in the process.

### Preparation 7

### 2-(2-(2-(4-(5-(Methylsulfonyl)-1,3,4-oxadiazol-2-yl)phenoxy)ethoxy)ethoxy)acetic acid

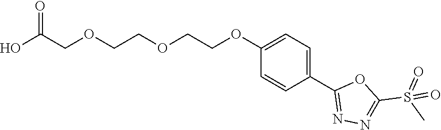 [SMILES: CS(=O)(=O)c1nnc(-c2ccc(OCCOCCOCC(=O)O)cc2)o1]

TFA (20 mL, 12.0 mmol) was added to a solution of tert-butyl 2-(2-(2-(4-(5 -(methylsulfonyl)-1,3,4-oxadiazol-2-yl)phenoxy)ethoxy)ethoxy)acetate (5.60 g, 12.0 mmol) in DCM (60 mL). The mixture was stirred at ambient temperature for 2 hours, concentrated in vacuo, and purified via silica gel column chromatography eluting with 0-100% EtOAc/DCM to give the title compound (4.12 g, 82%). ES/MS m/z: 387 (M+H).

### Preparation 8

### 2,5-Dioxopyrrolidin-1-yl-2-(2-(2-(4-(5-(methylsulfonyl)-1,3,4-oxadiazol-2-yl)phenoxy)ethoxy)ethoxy)acetate

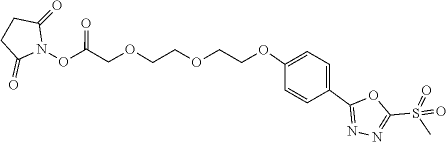 [SMILES: CS(=O)(=O)c1nnc(-c2ccc(OCCOCCOCC(=O)ON3C(=O)CCC3=O)cc2)o1]

EDCI (1.60 g, 10.3 mmol) was added to a solution of 2-(2-(2-(4-(5-(methylsulfonyl)-1,3,4-oxadiazol-2-yl)phenoxy)ethoxy)ethoxy)acetic acid (3.00 g, 7.38 mmol) and 1-hydroxypyrrolidine-2,5-dione (1.19 g, 10.3 mmol) in DCM (50 mL) and THF (70 mL). Another 20 mL of DCM was added to bring the mixture into a solution followed by stirring at ambient temperature for 12 hours. After this time, concentrated in vacuo and purified via silica gel column chromatography eluting with 0-100% EtOAc/DCM to give the title compound (2.61 g, 65%). ES/MS m/z: 484 (M+H).

The compound in Table 13 was prepared in a manner essentially analogous to that found in Preparation 8.

TABLE 13

| | | | ES/MS |
|---|---|---|---|
| Prep | Name | Structure | m/z |
| 9 | 2,5-Dioxopyrrolidin- 1-yl 2-(2-(2- (4-(5-(methylsulfonyl)- 1H- tetrazol-1- yl)phenoxy)ethoxy) ethoxy)acetate | 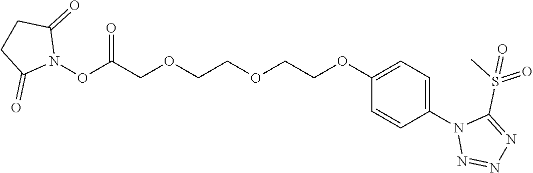 [SMILES: CS(=O)(=O)c1nnnn1-c1ccc(OCCOCCOCC(=O)ON2C(=O)CCC2=O)cc1] | 484 (M + H) |

### Preparation 10

### 2-(((2R,3R,4R,5R)-5-(2,4-Dioxo-3,4-dihydropyrimidin-1 (2H)-yl)-3-hydroxy-4-methoxytetrahydrofuran-2-yl)methyl) isoindoline-1,3-dione

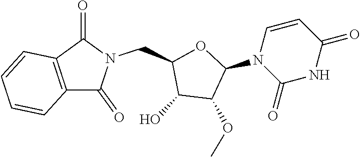 [SMILES: CO[C@@H]1[C@H](O)[C@@H](CN2C(=O)c3ccccc3C2=O)O[C@H]1n1ccc(=O)[nH]c1=O]

A solution of 1-((2R,3R,4R,5R)-4-hydroxy-5-(hydroxymethyl)-3-methoxytetrahydrofuran-2-yl)pyrimidine-2,4 (1H,3H)-dione (30 g, 120 mmol), isoindoline-1,3-dione (21 g, 140 mmol), DIAD (27 mL, 140 mmol), tributyl phosphine (36 mL, 150 mmol), and THF (300 mL) was stirred at ambient temperature for 12 h. The crude reaction was filtered, concentrated in vacuo, and purified via silica gel flash chromatography eluting with 0-100% EtOAc/hexanes to give the title compound as a white solid (6.0 g, 13%).

### Preparation 11

### 2-Cyanoethyl ((2R,3R,4R,5R)-5-(2,4-dioxo-3,4-dihydropyrimidin-1 (2H)-yl)-2-((1,3 -dioxoisoindolin-2-yl)methyl)-4-methoxytetrahydrofuran-3-yl) diisopropylphosphoramidite

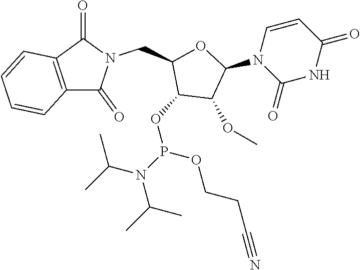 [SMILES: CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

A solution of 2-(((2R,3R,4R,5R)-5-(2,4-dioxo-3,4-dihydropyrimidin-1 (2H)-yl)-3-hydroxy-4-methoxytetrahydrofuran-2-yl)methyl) isoindoline-1,3-dione (3.00 g, 7.74 mmol), 2-cyanoethyl-N,N-diisopropylchlorophosphoramidite (2.47 mL, 11.6 mmol), DIEA (4.05 mL, 23.2 mmol), and DCM (40 mL) was stirred at ambient temperature. After 1 hour, additional 2-cyanoethyl-N,N-diisopropylchlorophosphoramidite (0.82 mL, 3.8 mmol) was added. After 1 hour, the crude reaction was poured into a slurry of silica gel (15 g) in 30 mL of 1% TEA/DCM, concentrated in vacuo to a dry powder, and purified via silica gel flash chromatography eluting with 40-100% EtOAc/hexanes (0.5% TEA) to give the title compound as a white foam (3.70 g, 81%). ¹H NMR (d6-DMSO) d 11.4 (br s, 1H), 7.96-7.78 (m, 5H), 5.83 (dd, 1H), 5.71 (dd, 1H), 4.46-3.47 (m, 9H), 3.39 (s, 1.5H), 3.35 (s, 1.5H), 2.82-2.73 (m, 2H), 1.16-0.97 (m, 12H). 31P NMR (d6-DMSO) d 149.7, 149.4.

### Preparation 12

### 3′ Oxadiazole Linker-Functionalized Sense Strand

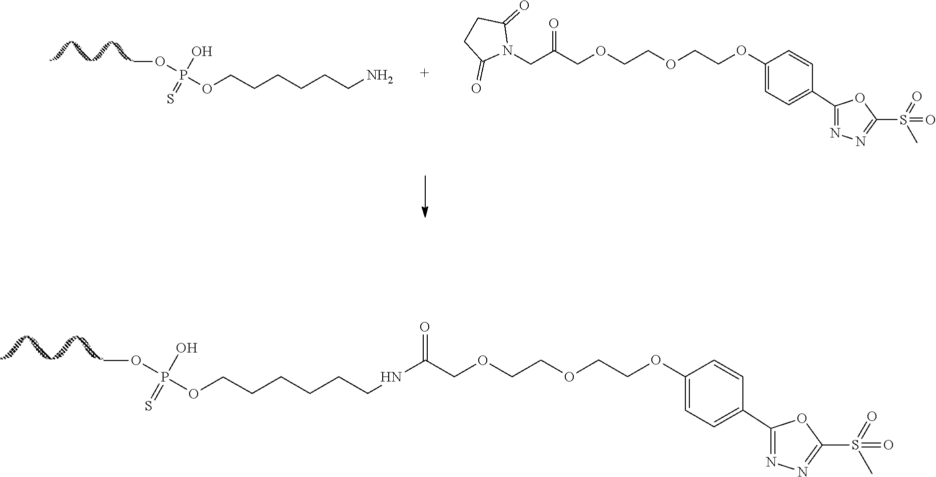 [SMILES: COP(O)(=S)OCCCCCCN ; CS(=O)(=O)c1nnc(-c2ccc(OCCOCCOCC(=O)CN3C(=O)CCC3=O)cc2)o1 ; COP(O)(=S)OCCCCCCNC(=O)COCCOCCOc1ccc(-c2nnc(S(C)(=O)=O)o2)cc1]

A 40 mL Falcon tube was charged with PRNP-SS-6aM (7.66 mg, 3.00 mL, 1.07 μmol) and 3 mL of PBS 7.4 (10×). 2,5-Dioxopyrrolidin-1-yl-2-(2-(2-(4-(5-(methylsulfonyl)-1,3,4-oxadiazol-2-yl)phenoxy)ethoxy)ethoxy)acetate (10.4 mg, 1.07 mL, 21.4 μmol) and ACN (1 mL) were added. The mixture was vortexed for 3 minutes, then shook at 900 rpm at ambient temperature for another hour. After this time, the mixture was concentrated in vacuo then de-salted using a 3K spin filter (Fisher biologics, 4500 rpm, 3× 1 hour). The optical density measurement of the product solution (average of 3 measurements, 20× dilution) was 6.42 OD/mL, concentration=628 μmol/L, 4.72 mg/mL, total 1.65 mL, 7.80 mg.

The compound in Table 14 below was prepared in a manner essentially analogous to that found in Preparation 12.

TABLE 14

| Prep | Name | Structure |
|---|---|---|
| 13 | 3′ Tetrazole-linked functionalized sense strand | 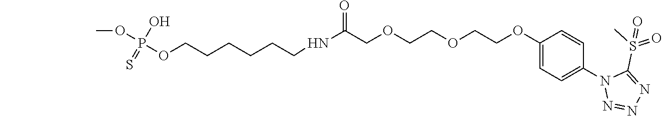 [SMILES: COP(O)(=S)OCCCCCCNC(=O)COCCOCCOc1ccc(-n2nnnc2S(C)(=O)=O)cc1] |

*12.85 OD/mL, concentration = 626 umol/L, 4.71 mg/mL, total 1.65 mL, 780 mg

### Preparation 14

### 5′ Oxadiazole Linker-Functionalized Sense Strand

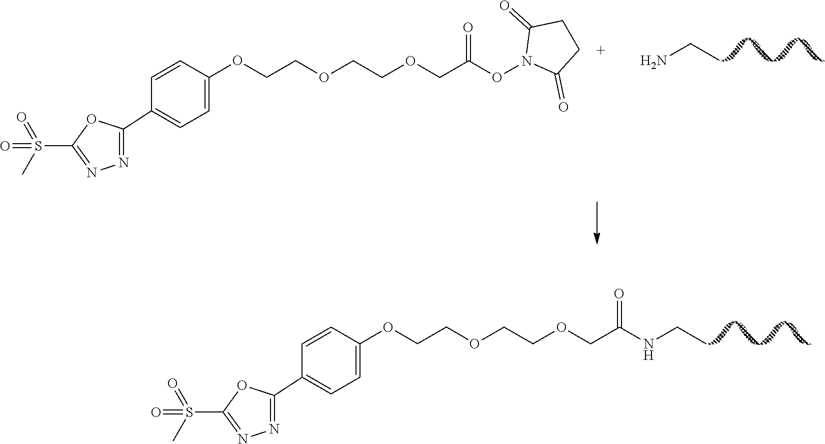

A sense strand (0.0013 mmol in 0.470 mL water) synthesized using conditions found in the protocols below was added to 20× borate buffer (0.071 mL), then was treated with a solution of 2,5-dioxopyrrolidin-1-yl-2-(2-(2-(4-(5-(methylsulfonyl)-1,3,4-oxadiazol-2-yl)phenoxy)ethoxy)ethoxy)acetate (0.0064 g, 0.0131 mmol) in MeCN (0.470 mL). The solution was shaken for 30 mins at 40° C. The solution was then diluted to 20 mL using RNAse free water to bring concentration of organic solvent to <10%. Excess NHS ester was removed using 20 mL 3K MWCO centrifugal spin tubes at 3500×g for ˜30 minutes. The oligonucleotides were rinsed with RNAse free water three times. After removing NHS ester, 1 mL of RNAse free water was added then aspirated 10× and the retentate was transferred to a 5 mL falcon tube. This was repeated until complete transfer of oligo by measuring concentration of compound on filter via nanodrop. The final oligonucleotide was analyzed for concentration (nano drop at A260), characterized by IP-RP, LCMS for mass purity, and UPLC for UV-purity. ES/MS (m/z): 7324.03 (M+H).

The compound in Table 15 below was prepared in a manner essentially analogous to that found in Preparation 14.

TABLE 15

| | | | ES/MS |
|---|---|---|---|
| Prep | Name | Structure | m/z |
| 15 | 5′ Tetrazole-linked functionalized sense strand | 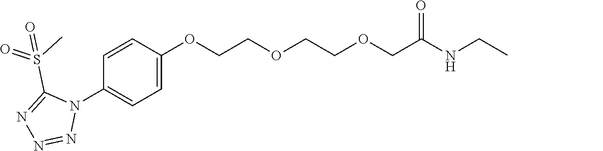 | 7324.04 (M + H) |

Linker-Functionalization of PRNP dsRNA

A freshly prepared solution of (2,5-dioxopyrrolidin-1-yl) 4-[(2,5-dioxopyrrol-1-yl)methyl]cyclohexanecarboxylate (185 mg, 0.542 mmol) in THF (50 mL) was added to PRNP_SS-3C6A (44 mL, 0.0528 mmol; OD/mL of 250.4, or ˜1200 μM (˜8.8 mg/mL)) in 0.2M phosphate buffer (44 mL). Vortexed vigorously for 2 minutes, and then shook at ambient temperature at 900 rpm for 2 h total. Analysis by LTQ showed about 94-95% conversion. Acidified to pH˜4 with 20-30 drops of 5N HC1, and then removed organics in a Genevac concentrator. Desalted by centrifugal filtration on a 3K spin filter (4×4000 rpm, 30 min), and pooled the retentates. The optical density measurement of the solution (average of 3 measurements, 10× dilution) was 266 equating to 1.3 mM and a total of 316 mg. Extinction coefficient was 204.12. LTQ/MS m/z 7358.

### Linker-PRNP Duplex

The nanodrop concentrations of aqueous solutions of each strand (average of 3×) were measure as SS=1322 μM and AS=1108 μM. The sense strand and antisense strand are annealed to form a dsRNA. 32 mL of SS and 36.2 mL of AS are mixed and shook for 30 min at 30° C. The amount of residual SS strand was measured until completion and required adding an additional 360 μL of AS. Removed endotoxins by filtering through a 0.45 μM filter. The resulting 75 mL of solution measured (Nanodrop™ Lite, 5× average, 10× dilution) 217 OD/mL equating to 575 μM and a total of 653 mg. LTQ/MS m/z 7358,7825; UV purity 99+%.

Conjugation of dsRNA to TfR Binding Proteins

Site-specific native or engineered cysteine amino acid residues in the TfR binding proteins were used to conjugate dsRNA. Cysteines can be engineered into the primary amino acid sequence of the TfR binding proteins. The approach of introducing cysteines as a means for conjugation has been described in WO 2018/232088, which is both incorporated by reference in its entirety and incorporated specifically in relation to conjugation via cysteine residues. For engineered cysteine conjugation, the TfR binding proteins were first reduced with 40 molar equivalents reducing agent dithiothreitol (DTT) at 37° C. for two hours, followed by desalting to remove reducing agent via dialysis or desalting columns. This is followed by re-oxidation of the TfR binding protein to reform the structural disulfides with 10 molar equivalent dehydroascorbic acid (DHAA) incubation at ambient temperature for two hours. A follow up desalting was performed to remove oxidizing agent.

Conjugation of dsRNA onto TfR binding proteins were done using the following methods.

### Conjugation Scheme 1

The first conjugation method utilized the 3′SS oxadiazole (OD)-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the OD-dsRNA with the TfR binding proteins at 1.2 to 2 molar equivalents for overnight conjugation at ambient temperature.

TfR Binding Protein Conjugation with 3′ OD Linker

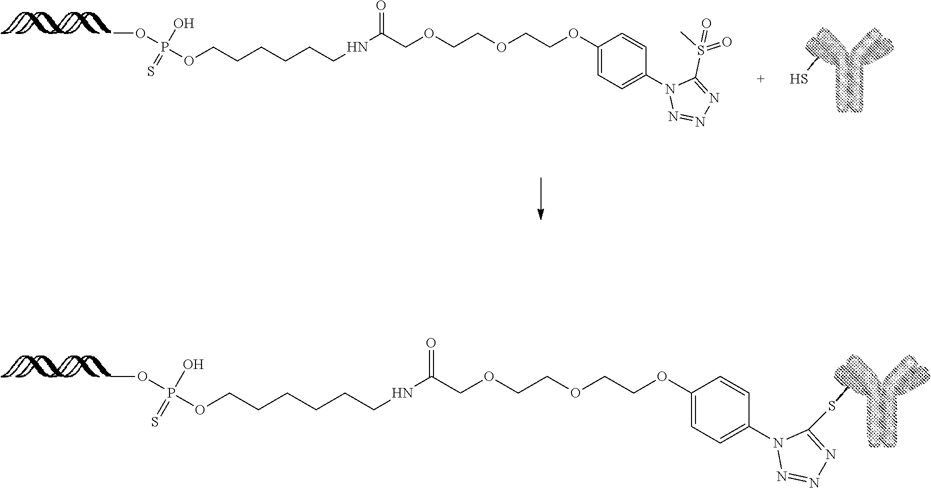

### Conjugation Scheme 2

The second conjugation method utilized the 5′SS oxadiazole (OD)-functionalized dsRNA for conjugating onto the engineered cysteine of the TfR binding proteins. For this method, TfR binding protein was prepared similarly as above to make the engineered thiol available for conjugation by undergoing a reduction and oxidation process of the TR binding proteins. This is followed by incubating the OD-dsRNA with the TfR binding proteins at 1.2 to 2 molar equivalents for overnight conjugation at ambient temperature.

TfR Binding Protein Conjugation with 5′ OD Linker

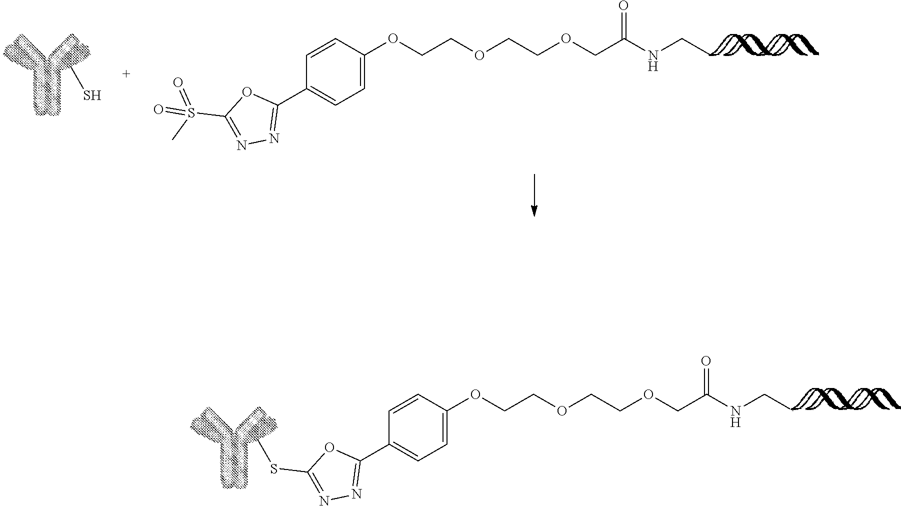

### Conjugation Scheme 3

The third conjugation method utilized the 3′SS tetrazole (MSPT)-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the MSPT-dsRNA with the TfR binding proteins at 1.2 to 2 molar equivalents for overnight conjugation at ambient temperature.

TfR Binding Protein Conjugation with 3′ MSPT Linker

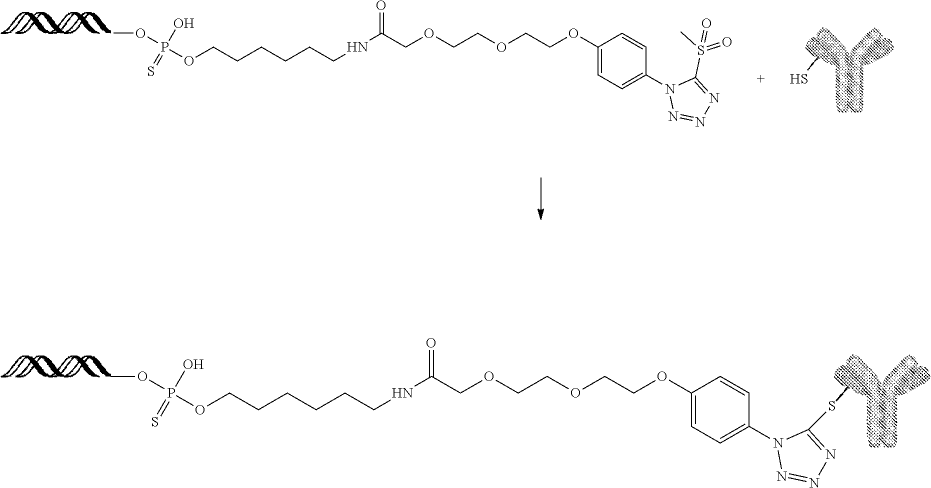

### Conjugation Scheme 4

The fourth conjugation method utilized the 5′SS tetrazole (MSPT)-functionalized dsRNA for conjugating onto the engineered cysteine of the TfR binding proteins. For this method, TfR binding protein was prepared similarly as above to make the engineered thiol available for conjugation by undergoing a reduction and oxidation process of the TR binding proteins. This is followed by incubating the MSPT-dsRNA with the TfR binding proteins at 1.2 to 2 molar equivalents for overnight conjugation at ambient temperature.

TfR Binding Protein Conjugation with 5′ MSPT Linker

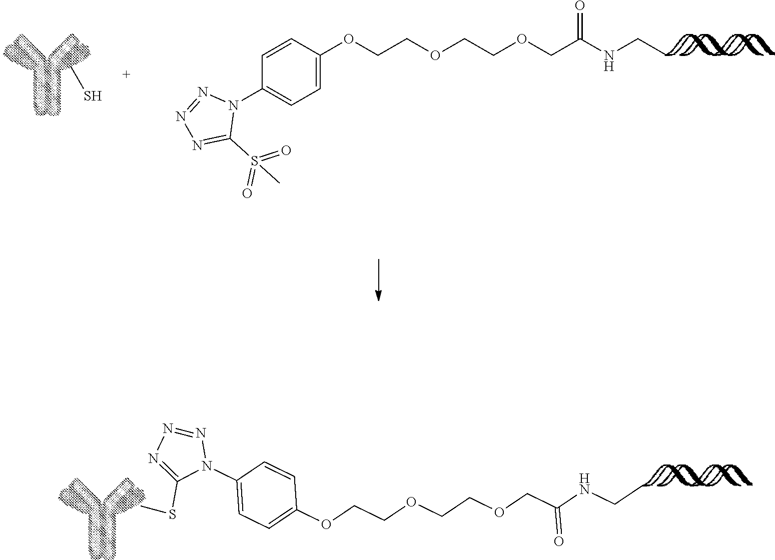

SMCC-Functionalization of dsRNA

To a 50 mL conical tube containing amino-functionalized sense strand oligonucleotide SS-PRNP-AMINO, for example SEQ ID 172 appended to a C6-amino chain via a 3′-terminal phosphorothiolate ester, as a solution in water (8.42 mL, 0.023 mmol, 19.857 mg/mL), was added sodium bicarbonate powder (59 mg, 0.702 mmol). The mixture was briefly vortexed and sonicated to dissolve the bicarbonate. A freshly prepared solution of (2,5-dioxopyrrolidin-1-yl) 4-[(2,5-dioxopyrrol-1-yl)methyl]cyclohexanecarboxylate (96 mg, 0.281 mmol) in acetonitrile (6.32 mL) was then added to the bicarbonate-oligo solution, for example, dsRNA-48-PS-C6-amino (8.42 mL, 0.023 mmol, 19.857 mg/mL in water) and vortexed for 30 seconds. Then, the reaction was allowed to proceed for 4 hours with shaking at ambient temperature at 300 rpm, at which point temperature control on a ThermoMixer® C took the reaction mixture down to 10° C. for 15 hours. At this point, LTQ-MS analysis indicated full conversion. The reaction was quenched to pH 5 using 1N HC1 (621 μL, 0.621 mmol). The quenched reaction mixture was then concentrated to approximately ½ volume using a GeneVac™ centrifugal evaporator and the resultant precipitate-containing suspension was filtered using a 0.22 micron Steri-Flip® apparatus to remove precipitate, rinsing once with 5 mL of nuclease-free water. The resulting clear solution containing oligo was then diluted to approximately 55 mL with 20% acetonitrile in nuclease-free water and concentrated using a CentriCon® ultrafiltration apparatus (3000 MWCO regenerated cellulose membrane). Following passage of all the volume through the Centricon®, two more 55 mL portions of 20% acetonitrile in nuclease-free water were passed through the CentriCon® to rinse the material, and finally one passage of 55 mL pure Milli-Q® water to remove residual acetonitrile. The retentate was then recovered by inverting the Centricon® apparatus on the included recovery cup. The Centricon® apparatus was then washed and aspirated twice with 800 μL nuclease-free water in each of the two filtration pores (1.6 mL total per wash), and the combined rinsate and retentate were passed through a 50k MWCO filter, which was rinsed one more time with 5 mL nuclease-free water. Finally, the desired compound was measured for concentration using a NanoDrop™ apparatus (OD260-calculated extinction coefficient: 216.09 mmol-1 cm-1) to give the desired compound (SEQ ID 172 with appended C6-amino-SMCC) as a solution of 9.77 mg/mL in 13.219 mL (129 mg, 68.1%). LTQ-MS: observed deconvoluted m/z=7361.7, calculated mass 7361.17, mass purity 91.37%.

### SMCC-dsRNA Duplex

To a conical tube containing SS-PRNP-AMINO-SMCC, with appended C6-Amino-SMCC (12.05 mL, 0.016 mmol, 1.328 mmol/L), was added its corresponding SS-PRNP-ANTISENSE, with 5′-E-vinyl phosphonate, (0.0165 mmol, 2.619 mmol/L). The solutions were shaken at 25° C. for 30 minutes to give the desired SMCC-functionalized dsRNA (SMCC-dsRNA), then refrigerated to 10° C. for storage. The annealed solutions were sampled for LTQ purity and UPLC non-denaturing chromatography. Analysis via non-denaturing UPLC (run at 10° C.) shows a major single peak of 92% purity. LTQ-MS: (Antisense strand observed deconvoluted m/z=7768.4, calculated 7769.04; Sense strand observed deconvoluted m/z=7360.4, calculated mass 7361.17).

### Conjugation Scheme for SMCC-dsRNA

The conjugation method utilized the SMCC-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the SMCC-dsRNA at 1.2 molar equivalents with the TfR binding proteins for overnight conjugation at room temperature.

Optionally, following conjugation a maleimide hydrolysis step can be done to secure the linker-payload in terminal stage and avoid deconjugation during human body circulation via retro-Michael addition. This succinimide ring hydrolysis process was done by elevating the conjugate pH to 9.0 using 50 mM Arginine (stock solution of 0.7M arginine, pH 9.0 was used) and incubating the solution at 37° C. for 20 hours. The hydrolysis state of the maleimide was confirmed by LCMS characterization of +18 Da that is incurred by the water addition to the succinimide ring.

Step 1a: TfR Binding Protein Conjugation with SMCC Linker

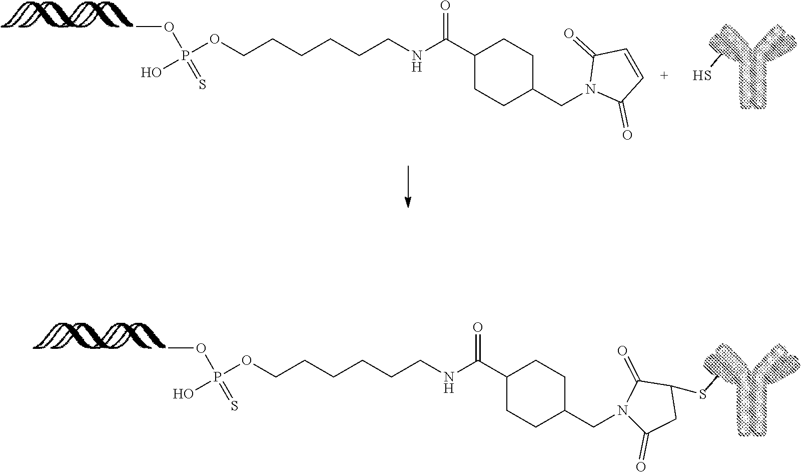

Step 1b: TfR Binding Protein Conjugation with SMCC Linker Ring Opening

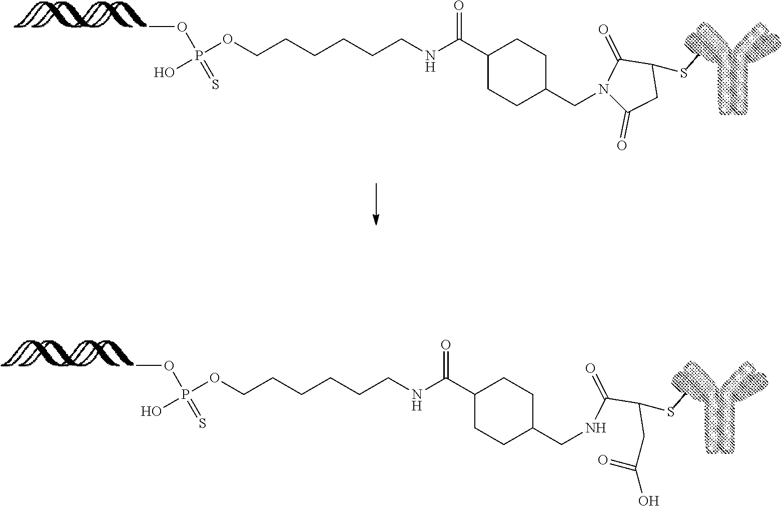 [SMILES: COP(O)(=S)OCCCCCCNC(=O)C1CCC(CN2C(=O)CC(S)C2=O)CC1 ; COP(O)(=S)OCCCCCCNC(=O)C1CCC(CNC(=O)C(S)CC(=O)O)CC1]

Synthesis of Mal-Tet-TCO and GDM linkers and conjugation of Mal-Tet-TCO- or GDM linker-functionalized dsRNA to the engineered cysteine of the TfR binding proteins have been described in WO 2024/036096.

Conjugation was monitored using analytical anion exchange chromatography. A ProPac™ SAX-10 HPLC Column, 10 μm particle, 4 mm diameter, 250 mmlength was utilized with the following method. Flow rate of 1 mL/min, Buffer A: 20 mM TRIS pH 7.0, Buffer B: 20 mM TRIS pH 7.0+1.5M NaCl, at 30° C.

Drug/siRNA to antibody/protein ratio (DAR) was calculated based on peak area % from the analytical anion exchange (aAEX) chromatogram.

Post conjugation of dsRNA to the TfR binding protein, excess dsRNA and unconjugated protein was removed by further purification. Either preparative size exclusion chromatography (SEC) or preparative anion exchange chromatography was utilized for purification of the final conjugate. Preparative SEC was performed using Cytiva Superdex® 200 in 1×PBS pH 7.2 under an isocratic condition. Alternatively, anion exchange, e.g., ThermoFisher POROS™ XQ, was used with starting buffer of 20 mM TRIS pH 7.0 and eluting with 20 column volume gradient with a buffer containing 20 mM TRIS pH 7.0 and 1M NaCl. These resulted in purified TfR binding protein-dsRNA conjugate devoid of excess dsRNA and minimal unconjugated protein. The resulting conjugate profile was analyzed by analytical anion exchange for final DAR quantitation (Table 16).

TABLE 16
siRNA/drug to TBP/antibody ratio (DAR)

| | Average | % of | % of | % of |
|---|---|---|---|---|
| | DAR | DAR0* | DAR1 | DAR2* |
| TBP5-SMCC- | 1 | 0 | 100 | 0 |
| dsRNA No. 2 | | | | |
| TBP5-MSPT- | 1 | 0 | 100 | 0 |
| dsRNA No. 2 | | | | |

*The percentage of DAR0 and DAR2 in this batch is <1%.

### Example 4: In Vitro Characterization of PRNP RNAi Agents

In vitro knockdown of PRNP by the PRNP RNAi agents was assessed in CHP-212 cells and SK—N—SH cells.

CHP-212 Cell Culture and RNAi Treatment and Analysis: CHP-212 cells (ATCC CRL-2273) were derived from the brain of a neuroblastoma patient. The base medium was composed of a 1:1 mixture of ATCC-formulated Eagle's Minimum Essential Medium, (Cat No. 30-2003), and F12 Medium. The complete growth medium was supplemented with 10% fetal bovine serum and 1× penicillin-streptomycin (Gibco) and cells incubated at 37° C. in a humidified atmosphere of 5% CO₂. On Day One, CHP-212 cells were plated in 96 well tissue culture plates and allowed to attach overnight. On Day Two, complete-media was replaced with RNAi agent in serum free media. Cells were incubated with RNAi agent for 72 hours at 37° C. in a humidified atmosphere of 5% CO₂. Analysis of changes in gene expression in RNAi treated CHP-212 cells was measured using Cells-to-Cr Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs00175591_m1 PRNP and ThermoFisher 4352934E GAPDH) were characterized with a dilution series of cDNA. RT-qPCR was performed in MicroAmp Optical 384-well reaction plates using the QuantStudio 7 Flex system. The delta-delta Ct method of normalizing to the housekeeping gene GAPDH was used to determine relative amounts of gene expression. GraphPad Prism v9.0 was used to determine IC50 with a three-parameter logistic fit. Results are shown in Table 17. IC50 potency of PRNP RNAi Agent No. 2 was 40.16 nM and maximal PRNP knock down at 1 μM was 77.52%.

TABLE 17
In vitro activity of PRNP RNAi agents in CHP-212 cells

| | | CHP-212, 3d |
|---|---|---|
| | CHP-212, 3d | % KD (knockdown) |
| | IC50 (nM) | of PRNP at 1 μM |
| PRNP RNAi Agent No. 2 | 40.16 | 77.52 |

### SK—N—SH Cell Culture and RNAi Treatment and Analysis:

SK—N—SH cells (ATCC HTB-11) were established from metastasized cells from the bone marrow of a four-year old patient. The base medium was composed of a 1:1 mixture of DMEM (Cat No. 11995) and F12 Media (Gibco Cat No. 11765047). The complete growth medium was supplemented with 1× Non-essential amino acids (Gibco Cat No. 11140050), 10% heat-inactivated fetal bovine serum and 1× penicillin-streptomycin (Gibco) and cells incubated at 37° C. in a humidified atmosphere of 5% CO₂. Cells were reverse transfected. On Day One, oligonucleotide libraries were complexed for 10 minutes with lipofectamine RNAiMax (Invitrogen Cat No. 13778100) in a 1:50 dilution with 0.5 μL of RNAiMax per well in 96 well format. SK—N—SH cells were plated on top of oligo-lipofectamine complexes in 96 well tissue culture plates and treated for 24 hours. On Day Two, analysis of changes in gene expression in RNAi treated SK—N—SH cells was measured using Cells-to-Cr Kits following the manufacturer's protocol (ThermoFisher A35377). Predesigned gene expression assays (supplied as 40× mixtures) were selected from Integrated DNA Technologies (IDT) (Coralville, IA, USA). The efficiencies of these assays (IDT Hs.PT.58.4310363 PRNP and ThermoFisher Hs99999904_m1 PPIA) were multiplexed and characterized with a dilution series of cDNA. RT-qPCR was performed in MicroAmp Optical 384-well reaction plates using the QuantStudio 7 Flex system. The delta Ct method of normalizing to the housekeeping gene PPIA was used to determine relative amounts of gene expression. GraphPad Prism v9.0 was used to determine IC50 with a four-parameter logistic fit.

The IC50 of the exemplary PRNP RNAi agents are shown in Table 18.

TABLE 18
In vitro activity of PRNP RNAi agents in SK-N-SH cells

| PRNP RNAi | SK-N-SH |
|---|---|
| Agent No. | IC50 (pM) |
| 2 | 24.15 |
| 11 | 9.708 |
| 12 | 13.28 |
| 13 | 14.2 |
| 14 | 16.75 |
| 15 | 18.77 |
| 16 | 21.29 |
| 17 | 24.62 |
| 18 | 31.44 |

### Example 5. In Vivo Characterization of Human TBP-PRNP dsRNA Conjugates in Human TfR (hTfR) Transgenic Mice Against PRNP

To determine the efficacy of the human TfR binding proteins-dsRNA conjugates against PRNP, they were tested in human TfR transgenic knock-in mice where the extracellular domain of transferrin-receptor have been humanized. TBP5-SMCC-dsRNA No. 2 or TBP5-MSPT-dsRNA No. 2 conjugate was dosed intravenously in hTfR transgenic mice and compared to the PBS or NTC (nontargeting control) dosed control group (n=6 per group). To determine optimal dose for TBP5-SMCC-dsRNA No. 2, 0.3, 1, 3, 10, or 30 mg/kg (mpk) siRNA doses were administered once intravenously. 14 days following initial dosing, mice were euthanized under carbon monoxide and sacrificed, then hemibrain was collected and processed for assessment of gene expression changes using RT-qPCR. To determine efficacy for TBP5-MSPT-dsRNA No. 2 conjugate, it was dosed at either 3 mg/kg or 10 mg/kg. 3 mg/kg groups were dosed once, or once a week for 4 weeks or once every 3 days, 4 times. 10 mg/kg groups were dosed once. 28 days following initial dosing, mice were euthanized under carbon monoxide and sacrificed, then hemibrain was collected and processed for assessment of gene expression changes using RT-qPCR. For both MSPT and SMCC, mPRNP from IDT (Mm.PT.58.30458786) was used as the target primer and mGAPDH from IDT (Mm.PT.39a.1) was used as the housekeeping gene primer.

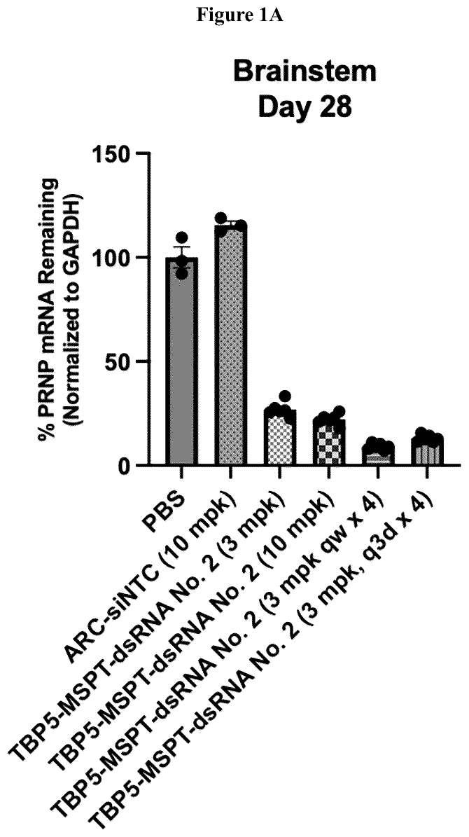

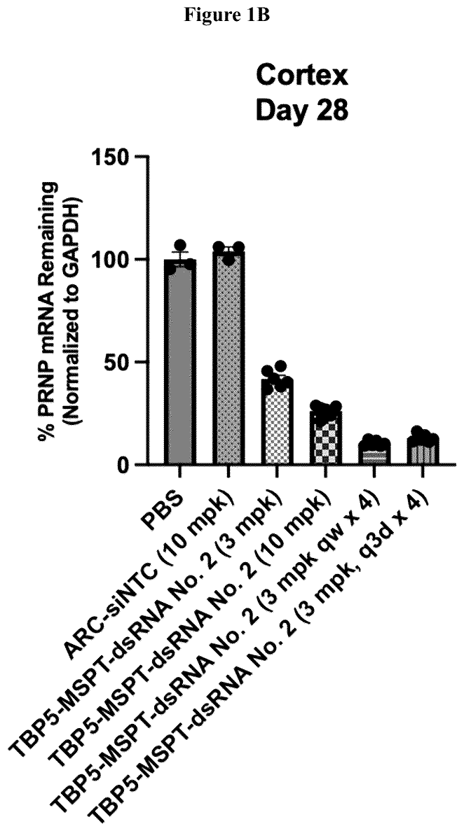

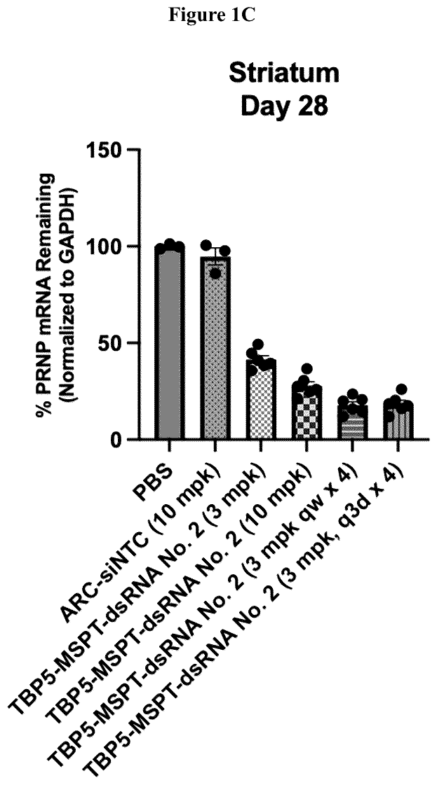

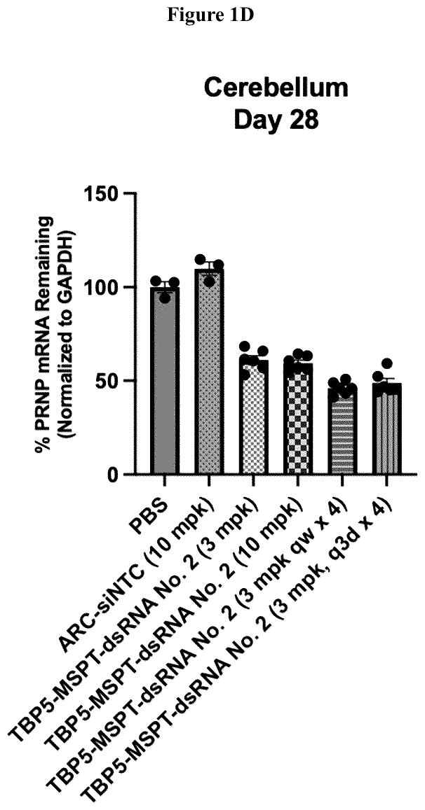

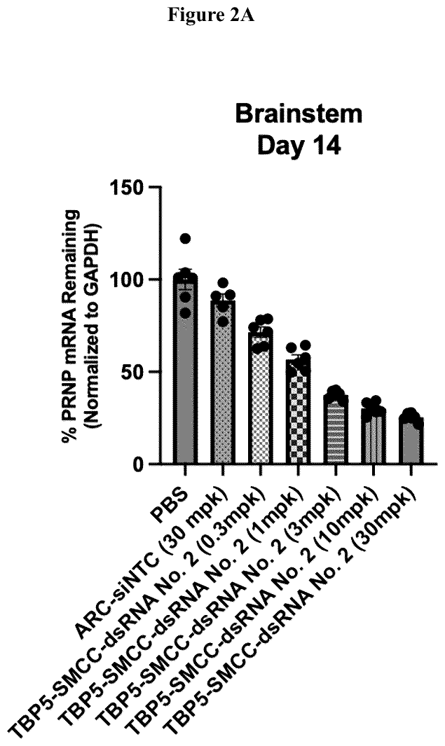

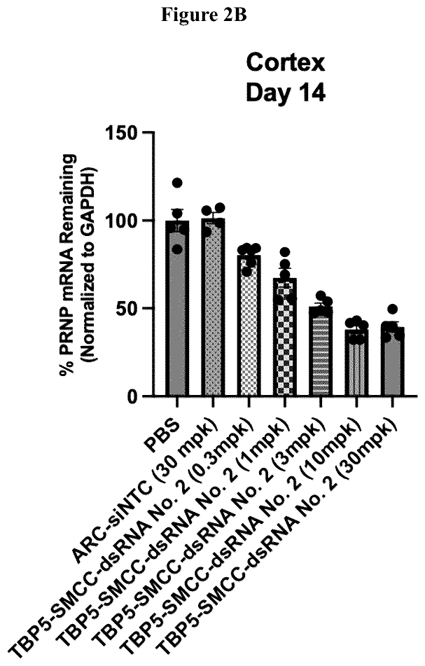

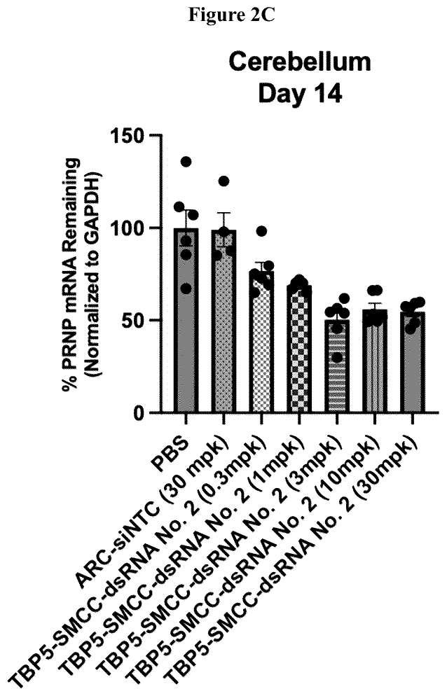

FIGS. 1A-1D show TBP5-MSPT-dsRNA No. 2 conjugate significant reduced PRNP mRNA in the key brain regions, including cortex, brainstem, striatum, and cerebellum, when compared to the PBS or nontargeting control (NTC) groups. FIGS. 2A-2C show TBP5-SMCC-dsRNA No. 2 conjugate successfully reduced PRNP mRNA in the key brain regions, including cortex, brainstem, and cerebellum, across the different doses tested.

### Example 6. In Vivo Characterization of Human TBP-PRNP dsRNA Conjugates in Human (hPRNP) Transgenic Mice

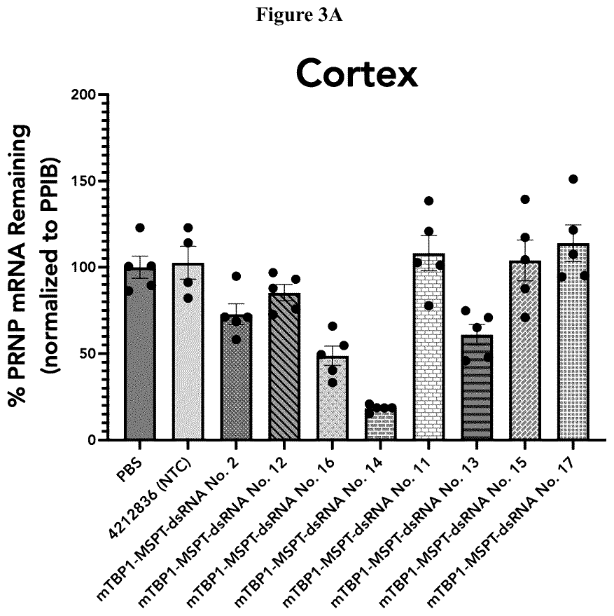

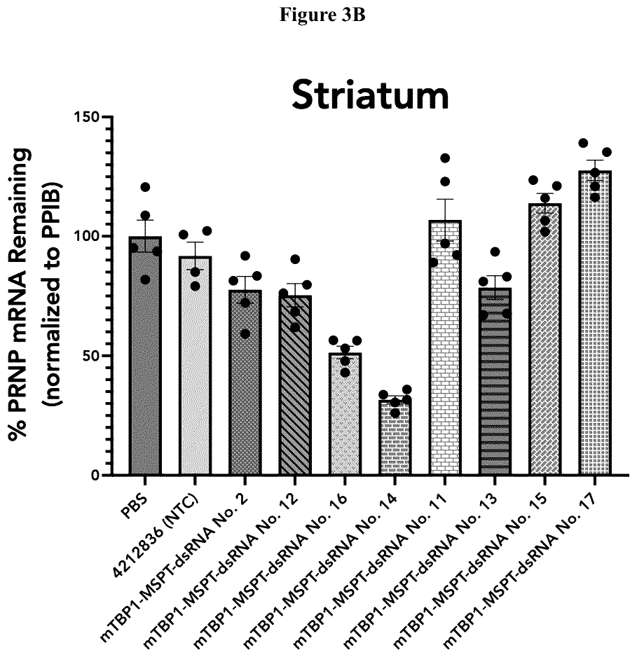

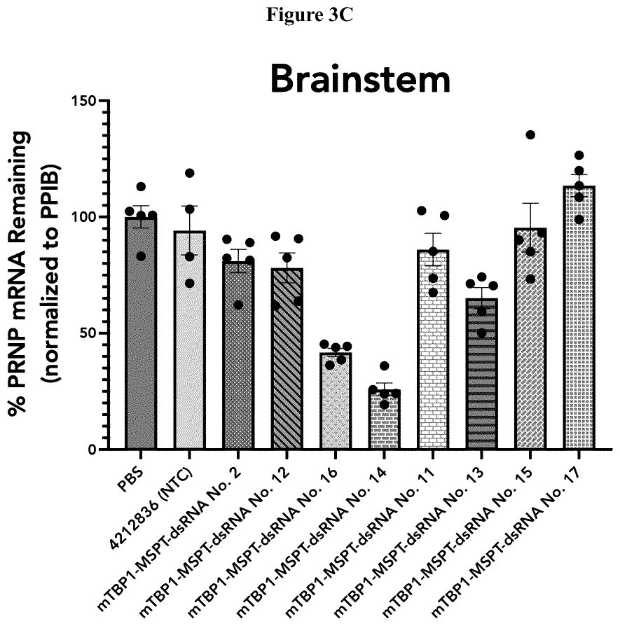

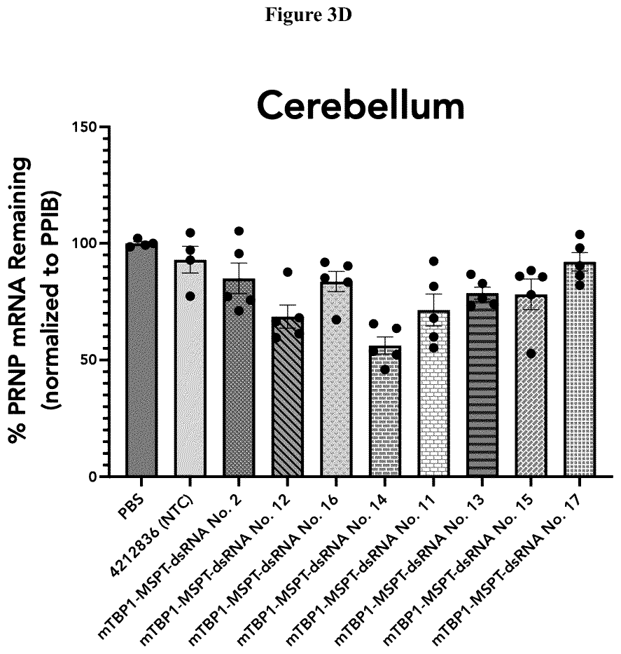

To determine the efficacy of the human TfR binding proteins-PRNP dsRNA conjugates, they were tested in knockout of endogenous human PRNP in transgenic mice on a C₅₇BL/6 background. Selected mTBP1-MSPT-PRNP dsRNA conjugates were dosed intravenously in hPRNP transgenic mice and compared to the PBS dosed control group (n=4 per group). siRNA doses were administered once intravenously at 3 mg/kg. 14 days following initial dosing, mice were euthanized and sacrificed, then various brain regions were collected and processed for assessment of gene expression changes using RT-qPCR. Results are shown in FIGS. 3A-3D.

A dose response and durability study for mTBP1-MSPT-dsRNA No. 14 conjugate was performed and compared to mTBP1-MSPT-dsRNA No. 2. Mice were dosed according to Table 19.

At 14, 28, 42, and/or 56 days post dose, mice were euthanized and various brain regions were collected and processed for assessment of gene expression changes using RT-qPCR. For both the initial efficacy study and the dose response/durability study, hPRNP from ThermoFisher (Hs00175591_m1) was used as the target primer and mPpib from ThermoFisher (Mm00478295_m1) was used as the housekeeping gene primer.

TABLE 19
Dosing regimens for human TBP-PRNP dsRNA conjugates

| | Dosing | | Timepoint |
|---|---|---|---|
| Molecule | Regimen | Dose | (days) |
| mTBP1-MSPT-dsRNA No. 14 | Once | 2.4 mpk | 14, 28, 42, 56 |
| mTBP1-MSPT-dsRNA No. 14 | Once | 0.24, | 28 |
| | | 0.8 mpk | |
| mTBP1-MSPT-dsRNA No. 14 | Q1W (once | 2.4 mpk | 28 |
| | a week) × 4 | | |
| mTBP1-MSPT-dsRNA No. 2 | Q1W × 4 | 2.4 mpk | 28 |
| mTBP1-MSPT-dsRNA No. 2 | Once | 2.4 mpk | 42 |

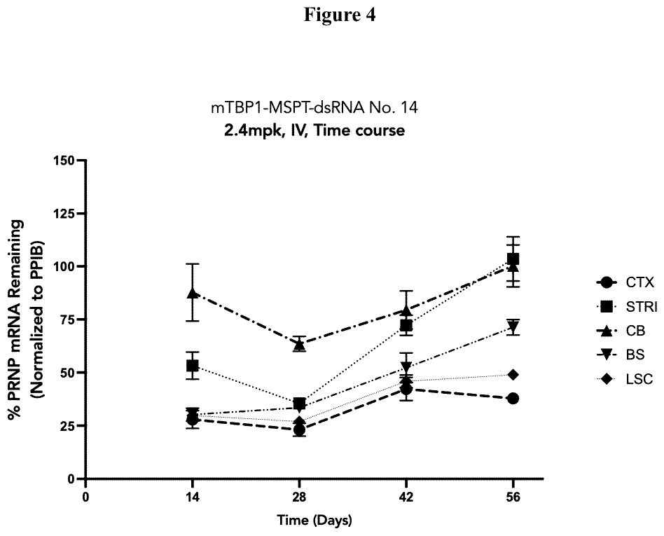

PRNP mRNA expression after treatment with the TBP-PRNP dsRNA conjugates are shown in Tables 20a and 20b and FIGS. 3-4.

TABLE 20a
PRNP mRNA expression after a single dose of TBP-PRNP dsRNA conjugate.
% PRNP mRNA Expression Remaining

| | Treatment | | | | | | | |
|---|---|---|---|---|---|---|---|---|
| | (QD × 1, Day 28 Necropsy) | | | | | | | |
| | Vehicle | | mTBP1-MSPT-dsRNA No. 14 | | | | | |
| | — | | 0.24 mg/kg | | 0.8 mg/kg | | 2.4 mg/kg | |
| | Mean | SD | Mean | SD | Mean | SD | Mean | SD |
| Tissue | (n = 3) | (n = 3) | (n = 4) | (n = 4) | (n = 4) | (n = 4) | (n = 4) | (n = 4) |
| Cortex | 100.0 | 23.21 | 64.0 | 9.02 | 39.6 | 2.68 | 23.1 | 6.17 |
| Cerebellum | 100.0 | 2.59 | 38.5 | 4.36 | 30.7 | 5.19 | 63.5 | 7.10 |
| Brainstem | 100.0 | 5.63 | 66.3 | 7.51 | 52.9 | 8.94 | 33.5 | 3.13 |
| Striatum | 100.0 | 3.97 | 77.1 | 11.40 | 52.8 | 8.49 | 35.5 | 4.32 |
| Lumbar | 100.0 | 16.92 | 54.1 | 8.64 | 43.7 | 10.44 | 27.0 | 3.51 |
| Spinal Cord | | | | | | | | |

TABLE 20b
PRNP mRNA expression after four
doses of TBP-PRNP dsRNA conjugate.
% PRNP mRNA Expression Remaining

| | Treatment | | | | | |
|---|---|---|---|---|---|---|
| | (QW × 4, Day 28 Necropsy) | | | | | |
| | | | mTBP1-MSPT- | | mTBP1-MSPT- | |
| | PBS | | dsRNA No. 14 | | dsRNA No. 2 | |
| | — | | 2.4 mg/kg | | 2.4 mg/kg | |
| | Mean | SD | Mean | SD | Mean | SD |
| Tissue | (n = 3) | (n = 3) | (n = 4) | (n = 4) | (n = 4) | (n = 4) |
| Cortex | 100.0 | 23.21 | 8.0 | 0.49 | 40.9 | 6.96 |
| Cerebellum | 100.0 | 2.59 | 42.6 | 6.28 | 80.3 | 20.03 |
| Brainstem | 100.0 | 5.63 | 16.3 | 4.41 | 41.5 | 4.51 |
| Striatum | 100.0 | 3.97 | 14.5 | 1.06 | 50.5 | 8.59 |
| Lumbar | 100.0 | 16.92 | 10.3 | 1.09 | 36.7 | 6.67 |
| Spinal Cord | | | | | | |

FIGS. 3A-3D show multiple TBP-MSPT-PRNP dsRNA conjugate successfully reduced PRNP mRNA in the key brain regions in mice, including cortex (3A), striatum (3B), brainstem (3C), and cerebellum (3D), when compared to PBS and NTC control groups. Table 20b shows mTBP1-MSPT-dsRNA No. 2 and mTBP1-MSPT-dsRNA No. 14 conjugates significantly reduced PRNP mRNA in the key brain regions of the mice, including cortex, striatum, cerebellum, and brainstem with a multi-dose regimen. FIG. 4 shows the durability of knock down of PRNP mRNA by TBP-MSPT-dsRNA No. 14 up to 56 days post-dose after a single injection. Evaluated regions include cortex (CTX), striatum (STRI), cerebellum (CB), brainstem (BS), and lumber spinal cord (LSC).

### Example 7. NHP Study of Human TBP-PRNP dsRNA Conjugates

To determine the efficacy of the human TfR binding proteins-PRNP dsRNA conjugates, they were tested in Cynomolgus macaques (NHPs). Selected TBP-MSPT-PRNP dsRNA conjugates were dosed once every 2 weeks by intravenous (Slow Bolus) injection in

Cynomolgus monkeys for 2 doses and compared to the PBS dosed control group (n=4 for vehicle control, n=2 for test groups). siRNA doses were administered once intravenously at 80 or 50 mg/kg, depending on the conjugate. At 28 days post dose, NHPs were euthanized, and various tissues were collected and processed for assessment of gene expression changes using RT-qPCR. For gene expression analysis by RT-qPCR, PRNP from ThermoFisher (Mf02826164_g1) was used as the target primer and PPIB and 18s rRNA from ThermoFisher (Mf02802985_m1 and Hs99999901_s1) were used as the housekeeping gene primers (geometric mean of the 2, AACT method).

Results are shown in Table 21.

TABLE 21
PRNP mRNA expression in NHP after
treatment of TBP- PRNP dsRNA conjugates.
% PRNP mRNA Expression Remaining

| | Treatment | | | | | |
|---|---|---|---|---|---|---|
| | (Q2W × 2, Day 29 Necropsy) | | | | | |
| | | | TBP5-MSPT- | | TBP8-MSPT- | |
| | PBS | | dsRNA No. 14 | | dsRNA No. 14 | |
| | — | | 80 mg/kg | | 50 mg/kg | |
| | Mean | SD | Mean | SD | Mean | SD |
| Tissue | (n = 4) | (n = 4) | (n = 2) | (n = 2) | (n = 2) | (n = 2) |
| Cortex | 100.0 | 40.5 | 16.1 | 7.8 | 7.5 | 2.6 |
| Cerebellum | 100.0 | 40.9 | 84.2 | 6.7 | 83.5 | 9.3 |
| Brainstem | 100.0 | 42.9 | 29.4 | 24.5 | 15.3 | 1.5 |
| Striatum | 100.0 | 31.1 | 30.4 | 12.2 | 19.9 | 6.8 |
| DRG - cervical | 100.0 | 21.3 | 24.6 | 3.1 | 27.3 | 13.8 |
| DRG - thoracic | 100.0 | 23.0 | 23.9 | 9.6 | 26.9 | 17.1 |
| DRG - lumbar | 100.0 | 65.9 | 23.5 | 18.1 | 18.0 | 1.4 |
| Liver | 100.0 | 59.1 | 50.0 | 1.1 | 39.5 | 22.9 |
| Gastrocnemius | 100.0 | 18.6 | 24.2 | 5.2 | 25.7 | 0.9 |
| Heart | 100.0 | 56.6 | 18.5 | 17.1 | 22.2 | 4.8 |
| Kidney | 100.0 | 10.9 | 93.5 | 39.0 | 102.2 | 32.2 |

Table 21 shows multiple TBP-MSPT-PRNP dsRNA conjugates successfully reduced PRNP mRNA in the key brain regions in NHP, including cortex, striatum, brainstem, and cerebellum, when compared to PBS controls with a multi-dose regimen.

SEQUENCE LISTING

| SEQ | |
|---|---|
| ID | |
| NO | Sequence |
| 1 | SYSMN |
| 2 | SISSSSSYIYYADSVKG |
| 3 | RHGYSNSDAFDN |
| 4 | RASQGISHYLV |
| 5 | AASSLQS |
| 6 | LQHNSYPWT |
| 7 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | S |
| 8 | DIQMTQSPSAMSASVGDRVTITCRASQGISHYLVWFQQKPGKVPKRLIYAASSLQSGVPSRF |
| | SGSGSGTEFTLTISSLQPEDFATYYCLQHNSYPWTFGQGTKVEIK |
| 9 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKRVEPKC |
| 10 | DIQMTQSPSAMSASVGDRVTITCRASQGISHYLVWFQQKPGKVPKRLIYAASSLQSGVPSRF |
| | SGSGSGTEFTLTISSLQPEDFATYYCLQHNSYPWTFGQGTKVEIKRTVAAPSVFIFPPSDEQL |
| | KSGTASVVCLLNNFYPREAKVQWKVDNALQSGNSQESVTEQDSKDSTYSLSSTLTLSKAD |
| | YEKHKVYACEVTHQGLSSPVTKSFNRGEC |
| 11 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPCVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKRVEPKCDKTHTGGGGQGGGGQGGGG |
| | QGGGGQGGGGQEVQLLESGGGLVQPGGSLRLSCAASGRYIDETAVAWFRQAPGKGREFV |
| | AGIGGGVDITYYADSVKGRFTISRDNSKNTLYLQMNSLRPEDTAVYYCGARPGRPLITSKV |
| | ADLYPYWGQGTLVTVSSPP |
| 12 | DIQMTQSPSAMSASVGDRVTITCRASQGISHYLVWFQQKPGKVPKRLIYAASSLQSGVPSRF |
| | SGSGSGTEFTLTISSLQPEDFATYYCLQHNSYPWTFGQGTKVEIKRTVAAPSVFIFPPSDEQL |
| | KSGTASVVCLLNNFYPREAKVQWKVDNALQCGNSQESVTEQDSKDSTYSLSSTLTLSKAD |
| | YEKHKVYACEVTHQGLSSPVTKSFNRGEC |
| 13 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPXVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSVFLF |
| | PPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVMHEALHNHYTQKSLSLSLG, wherein X is S or C. |
| 14 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSVFLF |
| | PPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVSTLPPSQEEMTKNQVS |
| | LMCLVYGFYPSDIXVEWESNGQPENNYKTTPPVLDSDGSFFLYSVLTVDKSRWQEGNVFSC |
| | SVMHEALHNHYTQKSLSLSLG, wherein X is A or C. |
| 15 | ESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAK |
| | GQPREPQVYTLPPSQGDMTKNQVQLTCLVKGFYPSDIXVEWESNGQPENNYKTTPPVLDSD |
| | GSFFLASRLTVDKSRWQEGNVFSCSVMHEALHNHYTQKSLSLSLG, wherein X is A or C. |
| 16 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPCVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSVFLF |
| | PPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVSTLPPSQEEMTKNQVS |
| | LMCLVYGFYPSDIAVEWESNGQPENNYKTTPPVLDSDGSFFLYSVLTVDKSRWQEGNVFSC |
| | SVMHEALHNHYTQKSLSLSLG |
| 17 | ESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAK |
| | GQPREPQVYTLPPSQGDMTKNQVQLTCLVKGFYPSDIAVEWESNGQPENNYKTTPPVLDSD |
| | GSFFLASRLTVDKSRWQEGNVFSCSVMHEALHNHYTQKSLSLSLG |
| 18 | QVQLVQSGAEVKKPGSSVKVSCKASGYTFSSYAIEWVRQAPGQGLEWMGGILPGSGTINY |
| | NEKFKGRVTITADKSTSTAYMELSSLRSEDTAVYYCARMSSNSDQGFDLWGQGTLVTVSS |
| | ASTKGPXVFPLAPCSRSTSESTAALGCLVKDYFPEPVTVSWNSGALTSGVHTFPAVLQSSGL |
| | YSLSSVVTVPSSSLGTKTYTCNVDHKPSNTKVDKRVESKYGPPCPPCPAPEAAGGPSVFLFP |
| | PKPKDTLMISRTPEVTCVVVDVSQEDPEVQFNWYVDGVEVHNAKTKPREEQFNSTYRVVS |
| | VLTVLHQDWLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFLLYSKLTVDKSRWQEGNVFSCS |
| | VMHEALHNHYTQKSLSLSLG, wherein X is S or C. |
| 19 | DIQMTQSPSSLSASVGDRVTITCKASQGISRFLSWFQQKPGKAPKSLIYAVSSLVDGVPSRFS |
| | GSGSGTDFTLTISSLQPEDFATYYCVQYNSYPYGFGGGTKVEIKRTVAAPSVFIFPPSDEQLK |
| | SGTASVVCLLNNFYPREAKVQWKVDNALQSGNSQESVTEQDSKDSTYSLSSTLTLSKADYE |
| | KHKVYACEVTHQGLSSPVTKSFNRGEC |
| 20 | ETAVA |
| 21 | GIGGGVDITYYADSVKG |
| 22 | RPGRPLITSKVADLYPY |
| 23 | EVQLLESGGGLVQPGGSLRLSCAASGRYIDETAVAWFRQAPGKGREFVAGIGGGVDITYYA |
| | DSVKGRFTISRDNSKNTLYLQMNSLRPEDTAVYYCGARPGRPLITSKVADLYPYWGQGTLV |
| | TVSSPP |
| 24 | GGGGQGGGGQGGGGQGGGGQ |
| 25 | CACGACUGCGUCAAUAUCACA |
| 26 | UGUGAUAUUGACGCAGUCGUGCA |
| 27 | AUUACUUUUCUGCAAUGUUAA |
| 28 | UUAACAUUGCAGAAAAGUAAUAC |
| 29 | UACAAUGUGCACUGAAUCGUA |
| 30 | UACGAUUCAGUGCACAUUGUAAG |
| 31 | GAAGUGGAAAAAGAAAUUCUA |
| 32 | UAGAAUUUCUUUUUCCACUUCAA |
| 33 | CACCAUCUUUCUAAUCUUUUA |
| 34 | UAAAAGAUUAGAAAGAUGGUGAA |
| 35 | CUAAUGCAUUAACUUUUGUAA |
| 36 | UUACAAAAGUUAAUGCAUUAGAC |
| 37 | GCUCAGUAUACUAAUGCCCUA |
| 38 | UAGGGCAUUAGUAUACUGAGCUC |
| 39 | UUCCCUGAAUUGUUUGAUAUA |
| 40 | UAUAUCAAACAAUUCAGGGAAUA |
| 41 | UGAUGUUUUACUUUUCACAGA |
| 42 | UCUGUGAAAAGUAAAACAUCACC |
| 43 | mC*mA*mCmGmAmCmUmGfCfGfUmCmAmAmUmAmUmCmA*mC*mA |
| 44 | mU*fG*mUmGmAmUmAmUmUmGmAmCmGfCmAfGmUmCmGmUmG*mC*mA |
| 45 | mA*mU*mUmAmCmUmUmUfUfCfUmGmCmAmAmUmGmUmU*mA*mA |
| 46 | mU*fU*mAmAmCmAmUmUmGmCmAmGmAfAmAfAmGmUmAmAmU*mA*mC |
| 47 | mU*mA*mCmAmAmUmGmUfGfCfAmCmUmGmAmAmUmCmG*mU*mA |
| 48 | mU*fA*mCmGmAmUmUmCmAmGmUmGmCfAmCfAmUmUmGmUmA*mA*mG |
| 49 | mG*mA*mAmGmUmGmGmAfAfAfAmAmGmAmAmAmUmUmC*mU*mA |
| 50 | mU*fA*mGmAmAmUmUmUmCmUmUmUmUfUmCfCmAmCmUmUmC*mA*mA |
| 51 | mC*mA*mCmCmAmUmCmUfUfUfCmUmAmAmUmCmUmUmU*mU*mA |
| 52 | mU*fA*mAmAmAmGmAmUmUmAmGmAmAfAmGfAmUmGmGmUmG*mA*mA |
| 53 | mC*mU*mAmAmUmGmCmAfUfUfAmAmCmUmUmUmUmGmU*mA*mA |
| 54 | mU*fU*mAmCmAmAmAmAmGmUmUmAmAfUmGfCmAmUmUmAmG*mA*mC |
| 55 | mG*mC*mUmCmAmGmUmAfUfAfCmUmAmAmUmGmCmCmC*mU*mA |
| 56 | mU*fA*mGmGmGmCmAmUmUmAmGmUmAfUmAfCmUmGmAmGmC*mU*mC |
| 57 | mU*mU*mCmCmCmUmGmAfAfUfUmGmUmUmUmGmAmUmA*mU*mA |
| 58 | mU*fA*mUmAmUmCmAmAmAmCmAmAmUfUmCfAmGmGmGmAmA*mU*mA |
| 59 | mU*mG*mAmUmGmUmUmUfUfAfCmUmUmUmUmCmAmCmA*mG*mA |
| 60 | mU*fC*mUmGmUmGmAmAmAmAmGmUmAfAmAfAmCmAmUmCmA*mC*mC |
| 61 | GCCAGTCGCT GACAGCCGCG GCGCCGCGAG CTTCTCCTCT CCTCACGACC GAGGCAGAGC |
| | AGTCATTATG GCGAACCTTG GCTGCTGGAT GCTGGTTCTC TTTGTGGCCA CATGGAGTGA |
| | CCTGGGCCTC TGCAAGAAGC GCCCGAAGCC TGGAGGATGG AACACTGGGG GCAGCCGATA |
| | CCCGGGGCAG GGCAGCCCTG GAGGCAACCG CTACCCACCT CAGGGCGGTG GTGGCTGGGG |
| | GCAGCCTCAT GGTGGTGGCT GGGGGCAGCC TCATGGTGGT GGCTGGGGGC AGCCCCATGG |
| | TGGTGGCTGG GGACAGCCTC ATGGTGGTGG CTGGGGTCAA GGAGGTGGCA CCCACAGTCA |
| | GTGGAACAAG CCGAGTAAGC CAAAAACCAA CATGAAGCAC ATGGCTGGTG CTGCAGCAGC |
| | TGGGGCAGTG GTGGGGGGCC TTGGCGGCTA CATGCTGGGA AGTGCCATGA GCAGGCCCAT |
| | CATACATTTC GGCAGTGACT ATGAGGACCG TTACTATCGT GAAAACATGC ACCGTTACCC |
| | CAACCAAGTG TACTACAGGC CCATGGATGA GTACAGCAAC CAGAACAACT TTGTGCACGA |
| | CTGCGTCAAT ATCACAATCA AGCAGCACAC GGTCACCACA ACCACCAAGG GGGAGAACTT |
| | CACCGAGACC GACGTTAAGA TGATGGAGCG CGTGGTTGAG CAGATGTGTA TCACCCAGTA |
| | CGAGAGGGAA TCTCAGGCCT ATTACCAGAG AGGATCGAGC ATGGTCCTCT TCTCCTCTCC |
| | ACCTGTGATC CTCCTGATCT CTTTCCTCAT CTTCCTGATA GTGGGATGAG GAAGGTCTTC |
| | CTGTTTTCAC CATCTTTCTA ATCTTTTTCC AGCTTGAGGG AGGCGGTATC CACCTGCAGC |
| | CCTTTTAGTG GTGGTGTCTC ACTCTTTCTT CTCTCTTTGT CCCGGATAGG CTAATCAATA |
| | CCCTTGGCAC TGATGGGCAC TGGAAAACAT AGAGTAGACC TGAGATGCTG GTCAAGCCCC |
| | CTTTGATTGA GTTCATCATG AGCCGTTGCT AATGCCAGGC CAGTAAAAGT ATAACAGCAA |
| | ATAACCATTG GTTAATCTGG ACTTATTTTT GGACTTAGTG CAACAGGTTG AGGCTAAAAC |
| | AAATCTCAGA ACAGTCTGAA ATACCTTTGC CTGGATACCT CTGGCTCCTT CAGCAGCTAG |
| | AGCTCAGTAT ACTAATGCCC TATCTTAGTA GAGATTTCAT AGCTATTTAG AGATATTTTC |
| | CATTTTAAGA AAACCCGACA ACATTTCTGC CAGGTTTGTT AGGAGGCCAC ATGATACTTA |
| | TTCAAAAAAA TCCTAGAGAT TCTTAGCTCT TGGGATGCAG GCTCAGCCCG CTGGAGCATG |
| | AGCTCTGTGT GTACCGAGAA CTGGGGTGAT GTTTTACTTT TCACAGTATG GGCTACACAG |
| | CAGCTGTTCA ACAAGAGTAA ATATTGTCAC AACACTGAAC CTCTGGCTAG AGGACATATT |
| | CACAGTGAAC ATAACTGTAA CATATATGAA AGGCTTCTGG GACTTGAAAT CAAATGTTTG |
| | GGAATGGTGC CCTTGGAGGC AACCTCCCAT TTTAGATGTT TAAAGGACCC TATATGTGGC |
| | ATTCCTTTCT TTAAACTATA GGTAATTAAG GCAGCTGAAA AGTAAATTGC CTTCTAGACA |
| | CTGAAGGCAA ATCTCCTTTG TCCATTTACC TGGAAACCAG AATGATTTTG ACATACAGGA |
| | GAGCTGCAGT TGTGAAAGCA CCATCATCAT AGAGGATGAT GTAATTAAAA AATGGTCAGT |
| | GTGCAAAGAA AAGAACTGCT TGCATTTCTT TATTTCTGTC TCATAATTGT CAAAAACCAG |
| | AATTAGGTCA AGTTCATAGT TTCTGTAATT GGCTTTTGAA TCAAAGAATA GGGAGACAAT |
| | CTAAAAAATA TCTTAGGTTG GAGATGACAG AAATATGATT GATTTGAAGT GGAAAAAGAA |
| | ATTCTGTTAA TGTTAATTAA AGTAAAATTA TTCCCTGAAT TGTTTGATAT TGTCACCTAG |
| | CAGATATGTA TTACTTTTCT GCAATGTTAT TATTGGCTTG CACTTTGTGA GTATTCTATG |
| | TAAAAATATA TATGTATATA AAATATATAT TGCATAGGAC AGACTTAGGA GTTTTGTTTA |
| | GAGCAGTTAA CATCTGAAGT GTCTAATGCA TTAACTTTTG TAAGGTACTG AATACTTAAT |
| | ATGTGGGAAA CCCTTTTGCG TGGTCCTTAG GCTTACAATG TGCACTGAAT CGTTTCATGT |
| | AAGAATCCAA AGTGGACACC ATTAACAGGT CTTTGAAATA TGCATGTACT TTATATTTTC |
| | TATATTTGTA ACTTTGCATG TTCTTGTTTT GTTATATAAA AAAATTGTAA ATGTTTAATA |
| | TCTGACTGAA ATTAAACGAG CGAAGATGAG CACCA |
| 62 | MANLGCWMLV LFVATWSDLG LCKKRPKPGG WNTGGSRYPG QGSPGGNRYP PQGGGGWGQP |
| | HGGGWGQPHG GGWGQPHGGG WGQPHGGGWG QGGGTHSQWN KPSKPKTNMK HMAGAAAAGA |
| | VVGGLGGYML GSAMSRPIIH FGSDYEDRYY RENMHRYPNQ VYYRPMDEYS NQNNFVHDCV |
| | NITIKQHTVT TTTKGENFTE TDVKMMERVV EQMCITQYER ESQAYYQRGS SMVLFSSPPV |
| | ILLISFLIFL IVG |
| 63 | HHHHHHCKRVEQKEECVKLAETEETDKSETMETEDVPTSSRLYWADLKTLLSEKLNSIEFA |
| | DTIKQLSQNTYTPREAGSQKDESLAYYIENQFHEFKFSKVWRDEHYVKIQVKSSIGQNMVTI |
| | VQSNGNLDPVESPEGYVAFSKPTEVSGKLVHANFGTKKDFEELSYSVNGSLVIVRAGEITFA |
| | EKVANAQSFNAIGVLIYMDKNKFPVVEADLALFGHAHLGTGDPYTPGFPSFNHTQFPPSQS |
| | SGLPNIPVQTISRAAAEKLFGKMEGSCPARWNIDSSCKLELSQNQNVKLIVKNVLKERRILNI |
| | FGVIKGYEEPDRYVVVGAQRDALGAGVAAKSSVGTGLLLKLAQVFSDMISKDGFRPSRSIIF |
| | ASWTAGDFGAVGATEWLEGYLSSLHLKAFTYINLDKVVLGTSNFKVSASPLLYTLMGKIM |
| | QDVKHPVDGKSLYRDSNWISKVEKLSFDNAAYPFLAYSGIPAVSFCFCEDADYPYLGTRLD |
| | TYEALTQKVPQLNQMVRTAAEVAGQLIIKLTHDVELNLDYEMYNSKLLSFMKDLNQFKTD |
| | IRDMGLSLQWLYSARGDYFRATSRLTTDFHNAEKTNRFVMREINDRIMKVEYHFLSPYVSP |
| | RESPFRHIFWGSGSHTLSALVENLKLRQKNITAFNETLFRNQLALATWTIQGVANALSGDIW |
| | NIDNEF |
| 64 | HHHHHHCKGVEPKTECERLAGTESPVREEPGEDFPAARRLYWDDLKRKLSEKLDSTDFTG |
| | TIKLLNENSYVPREAGSQKDENLALYVENQFREFKLSKVWRDQHFVKIQVKDSAQNSVIIV |
| | DKNGRLVYLVENPGGYVAYSKAATVTGKLVHANFGTKKDFEDLYTPVNGSIVIVRAGKIT |
| | FAEKVANAESLNAIGVLIYMDQTKFPIVNAELSFFGHAHLGTGDPYTPGFPSFNHTQFPPSRS |
| | SGLPNIPVQTISRAAAEKLFGNMEGDCPSDWKTDSTCRMVTSESKNVKLTVSNVLKEIKILN |
| | IFGVIKGFVEPDHYVVVGAQRDAWGPGAAKSGVGTALLLKLAQMFSDMVLKDGFQPSRSII |
| | FASWSAGDFGSVGATEWLEGYLSSLHLKAFTYINLDKAVLGTSNFKVSASPLLYTLIEKTM |
| | QNVKHPVTGQFLYQDSNWASKVEKLTLDNAAFPFLAYSGIPAVSFCFCEDTDYPYLGTTM |
| | DTYKELIERIPELNKVARAAAEVAGQFVIKLTHDVELNLDYERYNSQLLSFVRDLNQYRADI |
| | KEMGLSLQWLYSARGDFFRATSRLTTDFGNAEKTDRFVMKKLNDRVMRVEYHFLSPYVSP |
| | KESPFRHVFWGSGSHTLPALLENLKLRKQNNGAFNETLFRNQLALATWTIQGAANALSGD |
| | VWDIDNEF |
| 65 | HHHHHHHHGKPIPNPLLGLDSTGGGGSDSAQNSVIIVDKNGRLVYLVENPGGYVAYSKAA |
| | TVTGKLVHANFGTKKDFEDLYTPVNGSIVIVRAGKITFAEKVANAESLNAIGVLIYMDQTKF |
| | PIVNAELSFFGHAHLGGGGGGLPNIPVQTISRAAAEKLFGNMEGDCPSDWKTDSTCRMVTS |
| | ESKNVKLTVS |
| 66 | RHGYSNSDAFDT |
| 67 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDTWGQGTLVTVSS |
| 68 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDTWGQGTLVTVSS |
| | ASTKGPCVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKRV |
| 69 | DIQMTQSPSAMSASVGDRVTITCRASQGISHYLVWFQQKPGKVPKRLIYAASSLQSGVPSRF |
| | SGSGSGTEFTLTISSLQPEDFATYYCLQHNSYPWTFGQGTKVEIKRTVAAPSVFIFPPSDEQL |
| | KSGTASVVCLLNNFYPREAKVQWKVDNALQSGNSQESVTEQDSKDSTYSLSSTLTLSKAD |
| | YEKHKVYACEVTHQGLSSPVTKSFNRGE |
| 70 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDTWGQGTLVTVSS |
| | ASTKGPCVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKRVGGGGQGGGGQGGGGQGGGGQGGG |
| | GQEVQLLESGGGLVQPGGSLRLSCAASGRYIDETAVAWFRQAPGKGREFVAGIGGGVDITY |
| | YADSVKGRFTISRDNSKNTLYLQMNSLRPEDTAVYYCGARPGRPLITSKVADLYPYWGQG |
| | TLVTVSSPP |
| 71 | GSYWIC |
| 72 | CIYSTSGGRTYYASWVKG |
| 73 | GDDSISDAYFDL |
| 74 | QSSQSVYNNNRLA |
| 75 | DASTLAS |
| 76 | QGTYFSSGWSWA |
| 77 | QSLEESGGDLVKPEGSLTLTCTASGFSFSGSYWICWVRQAPGKGLEWIGCIYSTSGGRTYYA |
| | SWVKGRFTISKTSSTTVTLQMTSLTAADTATYFCARGDDSISDAYFDLWGPGTLVTVSS |
| 78 | ALDMTQTASPVSAAVGGTVTINCQSSQSVYNNNRLAWYQQKPGQPPKLLIYDASTLASGV |
| | PSRFKGSGSGTQFTLTISGVQSDDSATYYCQGTYFSSGWSWAFGGGTEVVVK |
| 79 | QSLEESGGDLVKPEGSLTLTCTASGFSFSGSYWICWVRQAPGKGLEWIGCIYSTSGGRTYYA |
| | SWVKGRFTISKTSSTTVTLQMTSLTAADTATYFCARGDDSISDAYFDLWGPGTLVTVSSAST |
| | KGPCVFPLAPCSRSTSESTAALGCLVKDYFPEPVTVSWNSGALTSGVHTFPAVLQSSGLYSL |
| | SSVVTVPSSSLGTKTYTCNVDHKPSNTKVDKRVESKYGPPCPPCPAPEAAGGPSVFLFPPKP |
| | KDTLMISRTPEVTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEGNVFSCSVM |
| | HEALHNHYTQKSLSLSLG |
| 80 | QSLEESGGDLVKPEGSLTLTCTASGFSFSGSYWICWVRQAPGKGLEWIGCIYSTSGGRTYYA |
| | SWVKGRFTISKTSSTTVTLQMTSLTAADTATYFCARGDDSISDAYFDLWGPGTLVTVSSAST |
| | KGPSVFPLAPCSRSTSESTAALGCLVKDYFPEPVTVSWNSGALTSGVHTFPAVLQSSGLYSL |
| | SSVVTVPSSSLGTKTYTCNVDHKPSNTKVDKRVESKYGPPCPPCPAPEAAGGPSVFLFPPKP |
| | KDTLMISRTPEVTCVVVDVSQEDPEVQFNWYVDGVEVHNAKTKPREEQFNSTYRVVSVLT |
| | VLHQDWLNGKEYKCKVSNKGLPSSIEKTISKAKGQPREPQVYTLPPSQEEMTKNQVSLTCL |
| | VKGFYPSDICVEWESNGQPENNYKTTPPVLDSDGSFFLYSRLTVDKSRWQEGNVFSCSVMH |
| | EALHNHYTQKSLSLSLG |
| 81 | ALDMTQTASPVSAAVGGTVTINCQSSQSVYNNNRLAWYQQKPGQPPKLLIYDASTLASGV |
| | PSRFKGSGSGTQFTLTISGVQSDDSATYYCQGTYFSSGWSWAFGGGTEVVVKRTVAAPSVF |
| | IFPPSDEQLKSGTASVVCLLNNFYPREAKVQWKVDNALQSGNSQESVTEQDSKDSTYSLSS |
| | TLTLSKADYEKHKVYACEVTHQGLSSPVTKSFNRGEC |
| 82 | ESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAK |
| | GQPREPQVYTLPPSQEEMTKNQVSLTCLVKGFYPSDIAVEWESNGQPENNYKTTPPVLDSD |
| | GSFLLYSKLTVDKSRWQEGNVFSCSVMHEALHNHYTQKSLSLSLG |
| 83 | ESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAK |
| | GQPREPQVYTLPPSQEEMTKNQVSLTCLVKGFYPSDICVEWESNGQPENNYKTTPPVLDSD |
| | GSFLLYSKLTVDKSRWQEGNVFSCSVMHEALHNHYTQKSLSLSLG |
| 84 | QSLEESGGDLVKPEGSLTLTCTASGFSFSGSYWICWVRQAPGKGLEWIGCIYSTSGGRTYYA |
| | SWVKGRFTISKTSSTTVTLQMTSLTAADTATYFCARGDDSISDAYFDLWGPGTLVTVSSAST |
| | KGPCVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTH |
| 85 | QSLEESGGDLVKPEGSLTLTCTASGFSFSGSYWICWVRQAPGKGLEWIGCIYSTSGGRTYYA |
| | SWVKGRFTISKTSSTTVTLQMTSLTAADTATYFCARGDDSISDAYFDLWGPGTLVTVSSAST |
| | KGPCVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHLEVLFQGPGGGGQGGGGQ |
| | GGGGQGGGGQGGGGQEVQLLESGGGLVQPGGSLRLSCAASGRYIDETAVAWFRQAPGKG |
| | REFVAGIGGGVDITYYADSVKGRFTISRDNSKNTLYLQMNSLRPEDTAVYYCAARPGRPLIT |
| | SKVADLYPYWGQGTLVTVSSPP |
| 86 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPCVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKRVEPKSSDKTHTGGGGQGGGGQGGG |
| | GQGGGGQGGGGQEVQLLESGGGLVQPGGSLRLSCAASGRYIDETAVAWFRQAPGKGREF |
| | VAGIGGGVDITYYADSVKGRFTISRDNSKNTLYLQMNSLRPEDTAVYYCGARPGRPLITSK |
| | VADLYPYWGQGTLVTVSSC |
| 87 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPCVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKRVEPKSCDKTHTGGGGQGGGGQGGG |
| | GQGGGGQGGGGQEVQLLESGGGLVQPGGSLRLSCAASGRYIDETAVAWFRQAPGKGREF |
| | VAGIGGGVDITYYADSVKGRFTISRDNSKNTLYLQMNSLRPEDTAVYYCGARPGRPLITSK |
| | VADLYPYWGQGTLVTVSSPP |
| 88 | EVQLVESGGGLVKPGGSLRLSCVASGFTFSSYSMNWVRQAPGKGLEWVSSISSSSSYIYYA |
| | DSVKGRFTISRDNAKNSLYLQMNSLRAEDTAVYYCARRHGYSNSDAFDNWGQGTLVTVS |
| | SASTKGPCVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKRVEPKSCD |
| 89 | DIQMTQSPSAMSASVGDRVTITCRASQGISHYLVWFQQKPGKVPKRLIYAASSLQSGVPSRF |
| | SGSGSGTEFTLTISSLQPEDFATYYCLQHNSYPWTFGQGTKVEIKRTVAAPSVFIFPPSDEQL |
| | KSGTASVVCLLNNFYPREAKVQWKVDNALQSGNSQESVTEQDSKDSTYSLSSTLTLSKAD |
| | YEKHKVYACEVTHQGLSSPVTKSFNRGEV |

1. A PRNP RNAi agent comprising Formula (I): (R-L)ₙ-P,
wherein R is a double stranded RNA (dsRNA) comprising a sense stand and an antisense strand, wherein the antisense strand is complementary to PRNP mRNA;
wherein L is a linker, or absent; and
wherein P is a protein comprising one monovalent human TfR binding domain,
wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3 or 66, LCDR1 comprises SEQ ID NO: 4, LCDR2 comprises SEQ ID NO: 5, and LCDR3 comprises SEQ ID NO: 6; and
wherein n is an integer of 1 to 3.

2. The PRNP RNAi agent of claim 1, wherein n is 1.

3. The PRNP RNAi agent of claim 1, wherein n is 2.

4. The PRNP RNAi agent of claim 1, wherein VH comprises SEQ ID NO: 7 or 67, and VL comprises SEQ ID NO: 8.

5. The PRNP RNAi agent of claim 1, wherein the human TfR binding domain is a Fab, scFv, Fv, or scFab.

6. The PRNP RNAi agent of claim 1, wherein the human TfR binding domain further comprises a heavy chain constant region comprising cysteine at residue 124 (according to the EU Index numbering).

7. The PRNP RNAi agent of claim 1, wherein P further comprises a half-life extender.

8. The PRNP RNAi agent of claim 7, wherein the half-life extender is an immunoglobulin Fc region or a VHH that binds human serum albumin (HSA).

9. The PRNP RNAi agent of claim 8, wherein the half-life extender is an immunoglobulin Fc region.

10. The PRNP RNAi agent of claim 9, wherein the immunoglobulin Fc region is a modified human IgG4 Fc region.

11. The PRNP RNAi agent of claim 10, wherein the modified human IgG4 Fc region comprises proline at residue 228, and alanine at residues 234 and 235 (all residues are numbered according to the EU Index numbering).

12. The PRNP RNAi agent of claim 9, wherein P comprises an immunoglobulin Fc region comprising cysteine at residue 378 (according to the EU Index numbering).

13. The PRNP RNAi agent of claim 9, wherein the immunoglobulin Fc region comprises:
(a) a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering); or
(b) a first Fc CH3 domain comprising leucine at residue 405, and a second Fc CH3 domain comprising arginine at residue 409 (all residues are numbered according to the EU Index numbering).

14. The PRNP RNAi agent of claim 1, wherein P comprises one heavy chain (HC) and one light chain (LC), wherein HC comprises SEQ ID NO: 9 and LC comprises SEQ ID NO: 10, 12, or 69.

15. The PRNP RNAi agent of claim 1, wherein P comprises one heavy chain (HC) and one light chain (LC), wherein HC comprises SEQ ID NO: 68 and LC comprises SEQ ID NO: 10, 12, or 69.

16. The PRNP RNAi agent of claim 1, wherein P comprises two heavy chains HC1 and HC2 and one light chain LC1, wherein HC1 comprises SEQ ID NO: 14, LC1 comprises SEQ ID NO: 10, HC2 comprises SEQ ID NO: 15.

17. The PRNP RNAi agent of claim 1, wherein P comprises two heavy chains HC1 and HC2 and one light chain LC1, wherein HC1 comprises SEQ ID NO: 16, LC1 comprises SEQ ID NO: 10, HC2 comprises SEQ ID NO: 17.

18. The PRNP RNAi agent of claim 8, wherein the half-life extender is a VHH that binds HSA.

19. The PRNP RNAi agent of claim 18, wherein the VHH comprises CDR1 comprising SEQ ID NO: 20, CDR2 comprising SEQ ID NO: 21, and CDR3 comprising SEQ ID NO: 22.

20. The PRNP RNAi agent of claim 18, wherein the VHH comprises SEQ ID NO: 23.

21. The PRNP RNAi agent of claim 18, wherein P comprises one heavy chain (HC) and one light chain (LC), and wherein the HC comprises SEQ ID NO: 11 and the LC comprises SEQ ID NO: 10, 12, or 69.

22. The PRNP RNAi agent of claim 18, wherein P comprises one heavy chain (HC) and one light chain (LC), and wherein the HC comprises SEQ ID NO: 70 and the LC comprises SEQ ID NO: 10, 12, or 69.

23. The PRNP RNAi agent of claim 18, wherein P comprises one heavy chain (HC) and one light chain (LC), and wherein the HC comprises SEQ ID NO: 87 and the LC comprises SEQ ID NO: 10.

24. The PRNP RNAi agent of claim 18, wherein P comprises one heavy chain (HC) and one light chain (LC), and wherein the HC comprises SEQ ID NO: 86 and the LC comprises SEQ ID NO: 89.

25. The PRNP RNAi agent of claim 1, wherein P is a heterodimeric antibody that comprises a first arm comprising one monovalent human TfR binding domain and a second arm that is a null arm.

26. The PRNP RNAi agent of claim 25, wherein the second arm comprises one heavy chain (HC) and one light chain (LC), and wherein the HC comprises SEQ ID NO: 18 and the LC comprises SEQ ID NO: 19.

27. The PRNP RNAi agent of claim 25, wherein P comprises two heavy chains HC1 and HC2 and two light chains LC1 and LC2, wherein HC1 comprises SEQ ID NO: 13, LC1 comprises SEQ ID NO: 10, HC2 comprises SEQ ID NO: 18, and LC2 comprises SEQ ID NO: 19.

28. The PRNP RNAi agent of claim 1, wherein L is a SMCC linker, OD linker, or MSPT linker.

29. The PRNP RNAi agent of claim 1, wherein L is a SMCC linker.

30. The PRNP RNAi agent of claim 1, wherein L is a MSPT linker.

31. The PRNP RNAi agent of claim 1, wherein P is linked to the 3′ end of the sense strand of dsRNA via the linker.

32. The PRNP RNAi agent of claim 1, wherein the sense strand and the antisense strand comprise a pair of nucleic acid sequences selected from the group consisting of:
(a) the sense strand comprises SEQ ID NO: 33, and the antisense strand comprises SEQ ID NO: 34,
(b) the sense strand comprises SEQ ID NO: 25, and the antisense strand comprises SEQ ID NO: 26,
(c) the sense strand comprises SEQ ID NO: 27, and the antisense strand comprises SEQ ID NO: 28,
(d) the sense strand comprises SEQ ID NO: 29, and the antisense strand comprises SEQ ID NO: 30,
(e) the sense strand comprises SEQ ID NO: 31, and the antisense strand comprises SEQ ID NO: 32,
(f) the sense strand comprises SEQ ID NO: 35, and the antisense strand comprises SEQ ID NO: 36,
(g) the sense strand comprises SEQ ID NO: 37, and the antisense strand comprises SEQ ID NO: 38,
(h) the sense strand comprises SEQ ID NO: 39, and the antisense strand comprises SEQ ID NO: 40, and
(i) the sense strand comprises SEQ ID NO: 41, and the antisense strand comprises SEQ ID NO: 42,
wherein optionally one or more nucleotides of the sense strand and the antisense strand are independently modified nucleotides, and wherein optionally one or more internucleotide linkages of the sense strand and the antisense strand are modified internucleotide linkages.

33. A PRNP RNAi agent comprising a double stranded RNA (dsRNA) comprising a sense stand and an antisense strand, wherein the sense strand and the antisense strand comprise a pair of nucleic acid sequences selected from the group consisting of:
(a) the sense strand comprises SEQ ID NO: 33, and the antisense strand comprises SEQ ID NO: 34,
(b) the sense strand comprises SEQ ID NO: 25, and the antisense strand comprises SEQ ID NO: 26,
(c) the sense strand comprises SEQ ID NO: 27, and the antisense strand comprises SEQ ID NO: 28,
(d) the sense strand comprises SEQ ID NO: 29, and the antisense strand comprises SEQ ID NO: 30,
(e) the sense strand comprises SEQ ID NO: 31, and the antisense strand comprises SEQ ID NO: 32,
(f) the sense strand comprises SEQ ID NO: 35, and the antisense strand comprises SEQ ID NO: 36,
(g) the sense strand comprises SEQ ID NO: 37, and the antisense strand comprises SEQ ID NO: 38,
(h) the sense strand comprises SEQ ID NO: 39, and the antisense strand comprises SEQ ID NO: 40, and
(i) the sense strand comprises SEQ ID NO: 41, and the antisense strand comprises SEQ ID NO: 42,
wherein optionally one or more nucleotides of the sense strand and the antisense strand are independently modified nucleotides, and wherein optionally one or more internucleotide linkages of the sense strand and the antisense strand are modified internucleotide linkages.

34. The PRNP RNAi agent of claim 32, wherein one or more nucleotides of the sense strand are modified nucleotides.

35. The PRNP RNAi agent of claim 34, wherein each nucleotide of the sense strand is a modified nucleotide.

36. The PRNP RNAi agent of claim 32, wherein one or more nucleotides of the antisense strand are modified nucleotides.

37. The PRNP RNAi agent of claim 36, wherein each nucleotide of the antisense strand is a modified nucleotide.

38. The PRNP RNAi agent of claim 32, wherein the modified nucleotide is a 2′-fluoro modified nucleotide, 2′-O-methyl modified nucleotide, 2′ deoxy nucleotide (DNA), or 2′-O—C₁₆ alkyl modified nucleotide.

39. The PRNP RNAi agent of claim 32, wherein the sense strand has four 2′-fluoro modified nucleotides at positions 7, 9, 10, and 11 from the 5′ end of the sense strand.

40. The PRNP RNAi agent of claim 39, wherein nucleotides at positions other than positions 7, 9, 10, and 11 of the sense strand are 2′-O-methyl modified nucleotides.

41. The PRNP RNAi agent of claim 32, wherein the antisense strand has four 2′-fluoro modified nucleotides at positions 2, 6, 14, and 16 from the 5′ end of the antisense strand.

42. The PRNP RNAi agent of claim 41, wherein nucleotides at positions other than positions 2, 6, 14 and 16 of the antisense strand are 2′-O-methyl modified nucleotides.

43. The PRNP RNAi agent of claim 32, wherein the sense strand has three 2′-fluoro modified nucleotides at positions 9, 10, and 11 from the 5′ end of the sense strand.

44. The PRNP RNAi agent of claim 43, wherein nucleotides at positions other than positions 9, 10, and 11 of the sense strand are 2′-O-methyl modified nucleotides.

45. The PRNP RNAi agent of claim 32, wherein the antisense strand has five 2′-fluoro modified nucleotides at positions 2, 5, 7, 14, and 16 from the 5′ end of the antisense strand.

46. The PRNP RNAi agent of claim 45, wherein nucleotides at positions other than positions 2, 5, 7, 14, and 16 of the antisense strand are 2′-O-methyl modified nucleotides.

47. The PRNP RNAi agent of claim 32, wherein the antisense strand has five 2′-fluoro modified nucleotides at positions 2, 5, 8, 14, and 16 from the 5′ end of the antisense strand.

48. The PRNP RNAi agent of claim 47, wherein nucleotides at positions other than positions 2, 5, 8, 14, and 16 of the antisense strand are 2′-O-methyl modified nucleotides.

49. The PRNP RNAi agent of claim 32, wherein the antisense strand has five 2′-fluoro modified nucleotides at positions 2, 3, 7, 14, and 16 from the 5′ end of the antisense strand.

50. The PRNP RNAi agent of claim 49, wherein nucleotides at positions other than positions 2, 3, 7, 14, and 16 of the antisense strand are 2′-O-methyl modified nucleotides.

51. The PRNP RNAi agent of claim 32, wherein the antisense strand has three 2′-fluoro modified nucleotides at positions 2, 14, and 16 from the 5′ end of the antisense strand.

52. The PRNP RNAi agent of claim 51, wherein nucleotides at positions other than positions 2, 14, and 16 of the antisense strand are 2′-O-methyl modified nucleotides.

53. The PRNP RNAi agent of claim 32, wherein the sense strand and the antisense strand have one or more modified internucleotide linkages.

54. The PRNP RNAi agent of claim 53, wherein the modified internucleotide linkage is phosphorothioate linkage.

55. The PRNP RNAi agent of claim 53, wherein the sense strand has four or five phosphorothioate linkages.

56. The PRNP RNAi agent of claim 53, wherein the antisense strand has four or five phosphorothioate linkages.

57. The PRNP RNAi agent of claim 32, wherein the antisense strand has a phosphate analog at the 5′ end.

58. The PRNP RNAi agent of claim 57, wherein the phosphate analog is 5′-vinylphosphonate.

59. The PRNP RNAi agent of claim 32, wherein the sense strand or antisense strand comprises an abasic moiety or inverted abasic moiety.

60. The PRNP RNAi agent of claim 32, wherein the sense strand and the antisense strand comprise a pair of nucleic acid sequences selected from the group consisting of:
(a) the sense strand comprises SEQ ID NO: 51, and the antisense strand comprises SEQ ID NO: 52,
(b) the sense strand comprises SEQ ID NO: 43, and the antisense strand comprises SEQ ID NO: 44,
(c) the sense strand comprises SEQ ID NO: 45, and the antisense strand comprises SEQ ID NO: 46,
(d) the sense strand comprises SEQ ID NO: 47, and the antisense strand comprises SEQ ID NO: 48,
(e) the sense strand comprises SEQ ID NO: 49, and the antisense strand comprises SEQ ID NO: 50,
(f) the sense strand comprises SEQ ID NO: 53, and the antisense strand comprises SEQ ID NO: 54,
(g) the sense strand comprises SEQ ID NO: 55, and the antisense strand comprises SEQ ID NO: 56,
(h) the sense strand comprises SEQ ID NO: 57, and the antisense strand comprises SEQ ID NO: 58, and
(i) the sense strand comprises SEQ ID NO: 59, and the antisense strand comprises SEQ ID NO: 60.

61. The PRNP RNAi agent of claim 32, wherein the sense strand and the antisense strand consist of a pair of nucleic acid sequences selected from the group consisting of:
(a) the sense strand consists of SEQ ID NO: 51, and the antisense strand consists of SEQ ID NO: 52,
(b) the sense strand consists of SEQ ID NO: 43, and the antisense strand consists of SEQ ID NO: 44,
(c) the sense strand consists of SEQ ID NO: 45, and the antisense strand consists of SEQ ID NO: 46,
(d) the sense strand consists of SEQ ID NO: 47, and the antisense strand consists of SEQ ID NO: 48,
(e) the sense strand consists of SEQ ID NO: 49, and the antisense strand consists of SEQ ID NO: 50,
(f) the sense strand consists of SEQ ID NO: 53, and the antisense strand consists of SEQ ID NO: 54,
(g) the sense strand consists of SEQ ID NO: 55, and the antisense strand consists of SEQ ID NO: 56,
(h) the sense strand consists of SEQ ID NO: 57, and the antisense strand consists of SEQ ID NO: 58, and
(i) the sense strand consists of SEQ ID NO: 59, and the antisense strand consists of SEQ ID NO: 60.

62. A pharmaceutical composition comprising the PRNP RNAi agent of claim 1 and a pharmaceutically acceptable carrier.

63. A method of treating a prion disease in a patient in need thereof, the method comprising administering to the patient an effective amount of the PRNP RNAi agent of claim 1.

64. The method of claim 63, wherein the prion disease is Creutzfeldt-Jakob disease, fatal familial insomnia, Gerstmann-Straussler disease, Huntington disease-like 1, or kuru.

65. The method of claim 63, wherein the PRNP RNAi agent is administered to the patient intravenously or subcutaneously.


SEQUENCE LISTING (89 sequences)

SEQ ID NO: 1 - protein, 5 aa, synthetic construct
SYSMN

SEQ ID NO: 2 - protein, 17 aa, synthetic construct
SISSSSSYIYYADSVKG

SEQ ID NO: 3 - protein, 12 aa, synthetic construct
RHGYSNSDAFDN

SEQ ID NO: 4 - protein, 11 aa, synthetic construct
RASQGISHYLV

SEQ ID NO: 5 - protein, 7 aa, synthetic construct
AASSLQS

SEQ ID NO: 6 - protein, 9 aa, synthetic construct
LQHNSYPWT

SEQ ID NO: 7 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 8 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 9 - protein, 223 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKC

SEQ ID NO: 10 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 381 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 12 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQCGNSQESVTEQDSKDSTYSLSSTLTLSKADYEKHKVYACEVTHQGLSSPVTKSFNRGEC

SEQ ID NO: 13 - protein, 447 aa, synthetic construct
  VARIANT 128: note = X is S or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 447 aa, synthetic construct
  VARIANT 379: note = X is A or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIXVEWESNGQPENNYKTTPPVLDSDGSFFLYSVLTVDKSRWQEGNVFSCSVMHEALHNHYTQKSLSLSLG

SEQ ID NO: 15 - protein, 228 aa, synthetic construct
  VARIANT 160: note = X is A or C
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIXVEWESNGQPENNYKTTPPVLDSDGSFFLASRLTVDKSRWQEGNVFSCSVMHEALHNHYTQKSLSLSLG

SEQ ID NO: 16 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 17 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 18 - protein, 446 aa, synthetic construct
  VARIANT 127: note = X is S or C
QVQLVQSGAEVKKPGSSVKVSCKASGYTFSSYAIEWVRQAPGQGLEWMGGILPGSGTINYNEKFKGRVTITADKSTSTAYMELSSLRSEDTAVYYCARMSSNSDQGFDLWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 19 - protein, 214 aa, synthetic construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 5 aa, synthetic construct
ETAVA

SEQ ID NO: 21 - protein, 17 aa, synthetic construct
GIGGGVDITYYADSVKG

SEQ ID NO: 22 - protein, 17 aa, synthetic construct
RPGRPLITSKVADLYPY

SEQ ID NO: 23 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 24 - protein, 20 aa, synthetic construct
GGGGQGGGGQGGGGQGGGGQ

SEQ ID NO: 25 - RNA, 21 nt, synthetic construct
  source 1..21: mol_type = other RNA
cacgactgcgtcaatatcaca

SEQ ID NO: 26 - RNA, 23 nt, synthetic construct
  source 1..23: mol_type = other RNA
tgtgatattgacgcagtcgtgca

SEQ ID NO: 27 - RNA, 21 nt, synthetic construct
  source 1..21: mol_type = other RNA
attacttttctgcaatgttaa

SEQ ID NO: 28 - RNA, 23 nt, synthetic construct
  source 1..23: mol_type = other RNA
ttaacattgcagaaaagtaatac

SEQ ID NO: 29 - RNA, 21 nt, synthetic construct
  source 1..21: mol_type = other RNA
tacaatgtgcactgaatcgta

SEQ ID NO: 30 - RNA, 23 nt, synthetic construct
  source 1..23: mol_type = other RNA
tacgattcagtgcacattgtaag

SEQ ID NO: 31 - RNA, 21 nt, synthetic construct
  source 1..21: mol_type = other RNA
gaagtggaaaaagaaattcta

SEQ ID NO: 32 - RNA, 23 nt, synthetic construct
  source 1..23: mol_type = other RNA
tagaatttctttttccacttcaa

SEQ ID NO: 33 - RNA, 21 nt, synthetic construct
  source 1..21: mol_type = other RNA
caccatctttctaatctttta

SEQ ID NO: 34 - RNA, 23 nt, synthetic construct
  source 1..23: mol_type = other RNA
taaaagattagaaagatggtgaa

SEQ ID NO: 35 - RNA, 21 nt, synthetic construct
  source 1..21: mol_type = other RNA
ctaatgcattaacttttgtaa

SEQ ID NO: 36 - RNA, 23 nt, synthetic construct
  source 1..23: mol_type = other RNA
ttacaaaagttaatgcattagac

SEQ ID NO: 37 - RNA, 21 nt, synthetic construct
  source 1..21: mol_type = other RNA
gctcagtatactaatgcccta

SEQ ID NO: 38 - RNA, 23 nt, synthetic construct
  source 1..23: mol_type = other RNA
tagggcattagtatactgagctc

SEQ ID NO: 39 - RNA, 21 nt, synthetic construct
  source 1..21: mol_type = other RNA
ttccctgaattgtttgatata

SEQ ID NO: 40 - RNA, 23 nt, synthetic construct
  source 1..23: mol_type = other RNA
tatatcaaacaattcagggaata

SEQ ID NO: 41 - RNA, 21 nt, synthetic construct
  source 1..21: mol_type = other RNA
tgatgttttacttttcacaga

SEQ ID NO: 42 - RNA, 23 nt, synthetic construct
  source 1..23: mol_type = other RNA
tctgtgaaaagtaaaacatcacc

SEQ ID NO: 43 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = cm
  modified_base 7: mod_base = um
  modified_base 8: mod_base = gm
  modified_base 9: mod_base = OTHER; note = 2'-fluorocytidine
  modified_base 10: mod_base = OTHER; note = 2'-fluoroguanosine
  modified_base 11: mod_base = OTHER; note = 2'-fluorouridine
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cacgactgcgtcaatatcaca

SEQ ID NO: 44 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = um
  modified_base 9: mod_base = um
  modified_base 10: mod_base = gm
  modified_base 11: mod_base = OTHER; note = 2'-O-methyladenosine
  modified_base 12: mod_base = cm
  modified_base 13: mod_base = gm
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = cm
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgtgatattgacgcagtcgtgca

SEQ ID NO: 45 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = um
  modified_base 7: mod_base = um
  modified_base 8: mod_base = um
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = gm
  modified_base 13: mod_base = cm
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = gm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
attacttttctgcaatgttaa

SEQ ID NO: 46 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = um
  modified_base 8: mod_base = um
  modified_base 9: mod_base = gm
  modified_base 10: mod_base = cm
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = cm
ttaacattgcagaaaagtaatac

SEQ ID NO: 47 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tacaatgtgcactgaatcgta

SEQ ID NO: 48 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = um
  modified_base 7: mod_base = um
  modified_base 8: mod_base = cm
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = um
  modified_base 18: mod_base = um
  modified_base 19: mod_base = gm
  modified_base 20: mod_base = um
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tacgattcagtgcacattgtaag

SEQ ID NO: 49 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = gm
  modified_base 7: mod_base = gm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gaagtggaaaaagaaattcta

SEQ ID NO: 50 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = um
  modified_base 8: mod_base = um
  modified_base 9: mod_base = cm
  modified_base 10: mod_base = um
  modified_base 11: mod_base = um
  modified_base 12: mod_base = um
  modified_base 13: mod_base = um
  modified_base 14: mod_base = OTHER; note = 2'-fluorouridine
  modified_base 15: mod_base = cm
  modified_base 16: mod_base = OTHER; note = 2'-fluorocytidine
  modified_base 17: mod_base = OTHER; note = 2'-O-methyladenosine
  modified_base 18: mod_base = cm
  modified_base 19: mod_base = um
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tagaatttctttttccacttcaa

SEQ ID NO: 51 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = um
  modified_base 7: mod_base = cm
  modified_base 8: mod_base = um
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = cm
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
caccatctttctaatctttta

SEQ ID NO: 52 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = OTHER; note = 2'-O-methyladenosine
  modified_base 8: mod_base = um
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = um
  modified_base 18: mod_base = gm
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
taaaagattagaaagatggtgaa

SEQ ID NO: 53 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = um
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroadenosine
  modified_base 12: mod_base = OTHER; note = 2'-O-methyladenosine
  modified_base 13: mod_base = cm
  modified_base 14: mod_base = um
  modified_base 15: mod_base = um
  modified_base 16: mod_base = um
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
ctaatgcattaacttttgtaa

SEQ ID NO: 54 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = um
  modified_base 11: mod_base = um
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = um
  modified_base 19: mod_base = um
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = cm
ttacaaaagttaatgcattagac

SEQ ID NO: 55 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = um
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = gm
  modified_base 17: mod_base = cm
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gctcagtatactaatgcccta

SEQ ID NO: 56 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = gm
  modified_base 5: mod_base = gm
  modified_base 6: mod_base = cm
  modified_base 7: mod_base = OTHER; note = 2'-O-methyladenosine
  modified_base 8: mod_base = um
  modified_base 9: mod_base = um
  modified_base 10: mod_base = OTHER; note = 2'-O-methyladenosine
  modified_base 11: mod_base = gm
  modified_base 12: mod_base = um
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = cm
tagggcattagtatactgagctc

SEQ ID NO: 57 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = cm
  modified_base 5: mod_base = cm
  modified_base 6: mod_base = um
  modified_base 7: mod_base = gm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorouridine
  modified_base 12: mod_base = gm
  modified_base 13: mod_base = um
  modified_base 14: mod_base = um
  modified_base 15: mod_base = um
  modified_base 16: mod_base = gm
  modified_base 17: mod_base = OTHER; note = 2'-O-methyladenosine
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
ttccctgaattgtttgatata

SEQ ID NO: 58 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = um
  modified_base 4: mod_base = OTHER; note = 2'-O-methyladenosine
  modified_base 5: mod_base = um
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = cm
  modified_base 11: mod_base = OTHER; note = 2'-O-methyladenosine
  modified_base 12: mod_base = OTHER; note = 2'-O-methyladenosine
  modified_base 13: mod_base = um
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = gm
  modified_base 18: mod_base = gm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tatatcaaacaattcagggaata

SEQ ID NO: 59 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = um
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = um
  modified_base 14: mod_base = um
  modified_base 15: mod_base = um
  modified_base 16: mod_base = cm
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tgatgttttacttttcacaga

SEQ ID NO: 60 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = um
  modified_base 4: mod_base = gm
  modified_base 5: mod_base = um
  modified_base 6: mod_base = gm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
tctgtgaaaagtaaaacatcacc

SEQ ID NO: 61 - RNA, 2435 nt, Homo sapiens
  source 1..2435: mol_type = mRNA
gccagtcgctgacagccgcggcgccgcgagcttctcctctcctcacgaccgaggcagagcagtcattatggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatgaggaaggtcttcctgttttcaccatctttctaatctttttccagcttgagggaggcggtatccacctgcagcccttttagtggtggtgtctcactctttcttctctctttgtcccggataggctaatcaatacccttggcactgatgggcactggaaaacatagagtagacctgagatgctggtcaagccccctttgattgagttcatcatgagccgttgctaatgccaggccagtaaaagtataacagcaaataaccattggttaatctggacttatttttggacttagtgcaacaggttgaggctaaaacaaatctcagaacagtctgaaatacctttgcctggatacctctggctccttcagcagctagagctcagtatactaatgccctatcttagtagagatttcatagctatttagagatattttccattttaagaaaacccgacaacatttctgccaggtttgttaggaggccacatgatacttattcaaaaaaatcctagagattcttagctcttgggatgcaggctcagcccgctggagcatgagctctgtgtgtaccgagaactggggtgatgttttacttttcacagtatgggctacacagcagctgttcaacaagagtaaatattgtcacaacactgaacctctggctagaggacatattcacagtgaacataactgtaacatatatgaaaggcttctgggacttgaaatcaaatgtttgggaatggtgcccttggaggcaacctcccattttagatgtttaaaggaccctatatgtggcattcctttctttaaactataggtaattaaggcagctgaaaagtaaattgccttctagacactgaaggcaaatctcctttgtccatttacctggaaaccagaatgattttgacatacaggagagctgcagttgtgaaagcaccatcatcatagaggatgatgtaattaaaaaatggtcagtgtgcaaagaaaagaactgcttgcatttctttatttctgtctcataattgtcaaaaaccagaattaggtcaagttcatagtttctgtaattggcttttgaatcaaagaatagggagacaatctaaaaaatatcttaggttggagatgacagaaatatgattgatttgaagtggaaaaagaaattctgttaatgttaattaaagtaaaattattccctgaattgtttgatattgtcacctagcagatatgtattacttttctgcaatgttattattggcttgcactttgtgagtattctatgtaaaaatatatatgtatataaaatatatattgcataggacagacttaggagttttgtttagagcagttaacatctgaagtgtctaatgcattaacttttgtaaggtactgaatacttaatatgtgggaaacccttttgcgtggtccttaggcttacaatgtgcactgaatcgtttcatgtaagaatccaaagtggacaccattaacaggtctttgaaatatgcatgtactttatattttctatatttgtaactttgcatgttcttgttttgttatataaaaaaattgtaaatgtttaatatctgactgaaattaaacgagcgaagatgagcacca

SEQ ID NO: 62 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 63 - protein, 681 aa, Mus musculus
HHHHHHCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 64 - protein, 678 aa, Homo sapiens
HHHHHHCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 65 - protein, 193 aa, Homo sapiens
HHHHHHHHGKPIPNPLLGLDSTGGGGSDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGGGGGGLPNIPVQTISRAAAEKLFGNMEGDCPSDWKTDSTCRMVTSESKNVKLTVS

SEQ ID NO: 66 - protein, 12 aa, synthetic construct
RHGYSNSDAFDT

SEQ ID NO: 67 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSS

SEQ ID NO: 68 - protein, 219 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 69 - protein, 213 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 70 - protein, 372 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 71 - protein, 6 aa, synthetic construct
GSYWIC

SEQ ID NO: 72 - protein, 18 aa, synthetic construct
CIYSTSGGRTYYASWVKG

SEQ ID NO: 73 - protein, 12 aa, synthetic construct
GDDSISDAYFDL

SEQ ID NO: 74 - protein, 13 aa, synthetic construct
QSSQSVYNNNRLA

SEQ ID NO: 75 - protein, 7 aa, synthetic construct
DASTLAS

SEQ ID NO: 76 - protein, 12 aa, synthetic construct
QGTYFSSGWSWA

SEQ ID NO: 77 - protein, 121 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSS

SEQ ID NO: 78 - protein, 112 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVK

SEQ ID NO: 79 - protein, 447 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 447 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFFLYSRLTVDKSRWQEGNVFSCSVMHEALHNHYTQKSLSLSLG

SEQ ID NO: 81 - protein, 219 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 83 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDICVEWESNGQPENNYKTTPPVLDSDGSFLLYSKLTVDKSRWQEGNVFSCSVMHEALHNHYTQKSLSLSLG

SEQ ID NO: 84 - protein, 228 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 85 - protein, 389 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLEVLFQGPGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 86 - protein, 381 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSC

SEQ ID NO: 87 - protein, 382 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 88 - protein, 225 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCD

SEQ ID NO: 89 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEV